(19) United States
(12) Patent Application Publication
(10) Pub. No.: US 2026/0258476 A1
(43) Pub. Date: Sep. 3, 2026

(54) IN SITU EPITRANSCRIPTOMIC PROFILING

(71) Applicants: The Broad Institute, Inc., Cambridge, MA (US); Massachusetts Institute of Technology, Cambridge, MA (US)

(72) Inventors: Xiao Wang, Cambridge, MA (US); Hu Zeng, Cambridge, MA (US); Jingyi Ren, Cambridge, MA (US); Jiakun Tian, Cambridge, MA (US); Jianting Guo, Cambridge, MA (US)

(73) Assignees: The Broad Institute, Inc., Cambridge, MA (US); Massachusetts Institute of Technology, Cambridge, MA (US)

(21) Appl. No.: 18/682,160

(22) PCT Filed: Aug. 10, 2022
(86) PCT No.: PCT/US2022/039895

§ 371 Date: Feb. 8, 2024

Related U.S. Application Data
(60) Provisional application No. 63/231,585, filed on Aug. 10, 2021.

(51) Int. Cl. C12Q 1/6813; C12Q 1/6855; C12Q 1/6869; C12Q 1/6883; C40B 70/00

(52) U.S. Cl. CPC C12Q 1/6813; C12Q 1/6855; C12Q 1/6869; C12Q 1/6883; C40B 70/00; C12Q 2600/112; C12Q 2600/154; C12Q 2600/166

(57) ABSTRACT

The present disclosure provides methods, compositions, and systems for profiling epitranscriptomic RNA modifications in a cell. The present disclosure also provides methods for profiling interactions between one or more RNAs of interest in a cell and an RNA-binding protein (e.g., a protein that introduces an epitranscriptomic modification on an RNA of interest). Also provided by the present disclosure are methods for diagnosing a disease or disorder in a subject based on a profile of epitranscriptomic RNA modifications or a profile of interactions between an RNA binding protein and RNAs in a cell, including cells within an intact tissue. Methods of screening for or testing a candidate agent capable of modulating epitranscriptomic modification of one or more RNAs or interactions between one or more RNAs and an RNA-binding protein are also provided by the present disclosure. The present disclosure also provides methods for treating a disease or disorder in a subject in need thereof. Pairs of probes and sets of probes comprising oligonucleotide portions, which may be useful for performing the methods described herein, are also described by the present disclosure. Additionally, the present disclosure provides kits comprising any of the probes described herein.

### RELATED APPLICATIONS

This application claims priority under 35 U.S.C. § 119(e) to U.S. Provisional Application, U.S. Ser. No. 63/231,585, filed Aug. 10, 2021, which is incorporated herein by reference.

### BACKGROUND OF THE INVENTION

Cellular RNAs contain a rich chemical repertoire of enzyme-catalyzed modifications (i.e., the epitranscriptome) (Roundtree, I. A. et al., Dynamic RNA modifications in gene expression regulation. Cell 169, 1187 (2017)). Genetic analyses of model organisms have revealed that RNA-modifying enzymes are vital to higher eukaryotes. Furthermore, RNA-modifying pathways have been identified to regulate cell fate, organ development, and human disease (e.g., cancer) (Jonkhout, N. et al., The RNA modification landscape in human disease. RNA 23, 1754 (2017); Li, X. et al., Epitranscriptome sequencing technologies: decoding RNA modifications. Nature Methods 14, 23 (2017)). Understanding of how these epitranscriptomic modifications impact cellular functions in complex biological systems, however, is still limited. This is because traditional analyses of RNA modifications rely on mixtures of millions of cells using bulk RNA sequencing or mass spectroscopy (Li, X. et al., Epitranscriptome sequencing technologies: decoding RNA modifications. Nature Methods 14, 23 (2017)). Yet, recent studies by single-cell sequencing approaches have revealed that multicellular organisms consist of diverse cell types, and even apparently homogenous cell populations have variable single-cell states. Moreover, the same type of RNA modification may have opposing regulatory effects dependent on cell type and physiological context. While single-cell RNA sequencing technology has been transformative for transcriptomic analysis, the lack of single-cell resolution and subcellular resolution in epitranscriptomic studies has obscured the heterogeneity of RNA modification patterns in biological tissues and impaired our ability to dissect how RNA modifications regulate gene expression across different cell types. Accordingly, systems for profiling epitranscriptomic RNA modifications at single-cell resolution and/or at subcellular resolution are needed.

### SUMMARY OF THE INVENTION

To address the limitations associated with previously developed single-cell RNA sequencing methods, platforms for three-dimensional (3D) in situ sequencing of RNA modifications, as well as platforms to profile interactions between RNAs and the proteins that install such RNA modifications, were developed (FIGS. 1, 10A, 12A, 13A, and 13C). These platforms can be utilized to analyze gene regulation mechanisms mediated by RNA modifications at single-cell or even subcellular resolution in intact biological tissues. Epitranscriptomic RNA modifications (e.g., N⁶-methyladenosine (m⁶A)) can thus be profiled, along with interactions between RNA-binding proteins (e.g., enzymes that install epitranscriptomic RNA modifications) and RNAs. The methods, probes, compositions, and systems disclosed herein are broadly applicable to the profiling of various epitranscriptomic RNA modifications or interactions between RNAs and RNA-binding proteins in various tissues. These methods and systems may also be useful for studying how epitranscriptomic RNA modifications or RNA-binding proteins in various cell types interact to define cell states and, for example, regulate brain function (Widagdo, J. et al., The m⁶A-epitranscriptomic signature in neurobiology: from neurodevelopment to brain plasticity. Journal of Neurochemistry 147, 137 (2018)). The methods, compositions, and systems described herein may also be useful for establishing new principles of post-transcriptional gene regulation mechanisms at single-cell or subcellular resolution (approximately 150-400 nm spatial resolution, depending on both the size of DNA amplicons and optical limits) in complex biological systems, as well as for the discovery of the roles of RNA chemical fingerprints (epitranscriptomic RNA modifications) in health and disease. The methods, compositions, and systems described herein may also be used on cells present within an intact tissue (e.g., a tissue sample provided by or from a human or non-human subject, such as a biopsy).

Thus, in one aspect, the present disclosure provides methods, compositions, and systems for profiling epitranscriptomic RNA modifications in a cell or multiple cells (see, for example, FIGS. 1, 10A, and 12A). In the methods and systems disclosed herein, a cell may be contacted with one or more pairs of probes or sets of probes, which are described further herein and may be used to amplify (e.g., by rolling circle amplification) epitranscriptomically modified RNAs of interest to produce one or more concatenated amplicons. The one or more concatenated amplicons may then be embedded in a polymeric matrix and sequenced to determine the identity of the transcripts and their location within the polymeric matrix (e.g., through SEDAL sequencing (Sequencing with Error-reduction by Dynamic Annealing and Ligation) as described further herein). Using the locations of the modified transcripts of interest, RNAs of interest comprising at least one epitranscriptomic modification may be profiled, and spatiotemporal information may be obtained to improve the understanding of how epitranscriptomic modification, subcellular location, and timing affect cellular function in health and disease. The methods and systems may be useful for comparing epitranscriptomic RNA modification in, for example, a cell (or multiple cells) from diseased and healthy tissue samples; or for comparing epitranscriptomic RNA modification in, for example, a cell treated with an agent (e.g., a therapeutic agent or potential therapeutic agent, such as a small molecule, a protein, a peptide, a nucleic acid, a lipid, or a carbohydrate) and an untreated cell, or a diseased cell and a healthy cell.

In some embodiments, the present disclosure provides methods for profiling epitranscriptomic RNA modifications in a cell comprising the steps of:
a) contacting the cell with one or more sets of probes, wherein each set of probes comprises a first probe (i.e., the “padlock probe”), a second probe (i.e., the “splint probe”), and a third probe (i.e., the “primer probe”) wherein:
   i) the first probe comprises an oligonucleotide portion that is complementary to a portion of the second probe, an oligonucleotide barcode sequence (e.g., a unique sequence used to identify each RNA of interest, for example, by SEDAL sequencing as discussed herein), an oligonucleotide portion that is complementary to an RNA of interest, and an oligonucleotide portion that is complementary to a portion of the third probe;
   ii) the second probe comprises a portion that recognizes an epitranscriptomic RNA modification and an oligonucleotide portion that is complementary to a portion of the first probe; and
   iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons embedded in the polymeric matrix to determine the identity and location of each epitranscriptomically modified RNA of interest in the cell.

In some embodiments, the present disclosure provides methods for profiling epitranscriptomic RNA modification in a cell comprising the steps of:
a) contacting the cell with one or more pairs of probes, wherein each pair of probes comprises a first probe (i.e., the “padlock probe”) and a second probe (i.e., the “primer probe”), wherein:
   i) the first probe comprises an oligonucleotide portion that is complementary to the second probe, an oligonucleotide portion that is complementary to an RNA of interest, and an oligonucleotide barcode sequence (e.g., a unique sequence used to identify each RNA of interest, for example, by SEDAL sequencing as discussed herein); and
   ii) the second probe comprises a portion that recognizes an epitranscriptomic RNA modification and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell. In certain embodiments in which only two probes are used in the methods herein, no splint probe is used.

These methods can thus be used to determine the locations of RNAs of interest modified with one or more epitranscriptomic modifications of interest within a cell or population of cells (e.g., in an intact tissue), or within organelles of a cell. In some embodiments, more than 1, more than 2, more than 3, more than 4, more than 5, more than 10, more than 20, more than 30, more than 40, more than 50, more than 100, more than 200, more than 500, more than 1000, more than 2000, or more than 3000 RNAs of interest are profiled simultaneously using the methods described herein. In some embodiments, the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), or 5-methylcytosine (m⁵C).

In another aspect, the present disclosure provides methods, probes, compositions, and systems for profiling interactions between RNA-binding proteins and RNAs in a cell or multiple cells, or in subcellular locations such as one or more particular organelles (see, for example, FIGS. 13A and 13C). In the methods and systems disclosed herein, a cell may be contacted with one or more pairs of probes or sets of probes, which are described further herein and may be used to amplify (e.g., by rolling circle amplification) RNAs of interest that are bound by RNA-binding proteins to produce one or more concatenated amplicons. The one or more concatenated amplicons may then be embedded in a polymeric matrix and sequenced to determine the identity of the transcripts and their location within the polymeric matrix (e.g., through SEDAL sequencing (Sequencing with Error-reduction by Dynamic Annealing and Ligation) as described further herein). Using the locations of the modified transcripts of interest, RNAs of interest bound by at least one RNA-binding protein may be profiled, and spatiotemporal information may be obtained to improve the understanding of how interactions between RNA-binding proteins and RNAs, the subcellular location of such interactions, and the timing of such interactions affect cellular function in health and disease. The methods and systems may be useful for comparing interactions between RNA-binding proteins and RNAs in, for example, a cell (or multiple cells) from diseased and healthy tissue samples; or for comparing interactions between RNA-binding proteins and RNAs in, for example, a cell treated with an agent (e.g., a therapeutic agent or potential therapeutic agent) and an untreated cell, or a diseased cell and a healthy cell.

In some embodiments, the present disclosure provides methods for profiling interactions between an RNA-binding protein and one or more RNAs of interest in a cell, the method comprising:
a) contacting the cell with one or more sets of probes, wherein each set of probes comprises a first probe (i.e., the “padlock probe”), a second probe (i.e., the “splint probe”), and a third probe (i.e., the “primer probe”) wherein:
   i) the first probe comprises an oligonucleotide portion that is complementary to a portion of the second probe, an oligonucleotide barcode sequence (e.g., a unique sequence used to identify each RNA of interest, for example, by SEDAL sequencing as discussed herein), an oligonucleotide portion that is complementary to an RNA of interest, and an oligonucleotide portion that is complementary to a portion of the third probe;
   ii) the second probe comprises a portion that recognizes an RNA-binding protein and an oligonucleotide portion that is complementary to a portion of the first probe; and
   iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each RNA of interest bound by an RNA-binding protein in the cell.

These methods can thus be used to determine the locations of RNAs of interest bound by one or more RNA-binding proteins (e.g., enzymes that install epitranscriptomic RNA modifications) within a cell or population of cells (e.g., in an intact tissue), or within organelles of a cell.

The methods, compositions, and systems described herein may be useful for studying epitranscriptomic RNA modification and interactions between RNA-binding proteins and RNAs in tissues (e.g., developing tissues, normal tissues, diseased tissues, treated tissues), for diagnosing and treating various diseases, for research purposes, and for drug discovery. Thus, in one aspect, the present disclosure provides methods for diagnosing a disease or disorder in a subject. For example, the methods for profiling epitranscriptomic RNA modifications or interactions between RNA-binding proteins and RNAs described herein may be performed on a cell, or on multiple cells, taken from a subject (e.g., a subject who is thought to have or is at risk of having a disease or disorder, or a subject who is healthy or thought to be healthy). The expression of various epitranscriptomically modified RNAs of interest or RNAs of interest bound by an RNA-binding protein in the cell can then be compared to the expression of the same modified RNAs of interest in a non-diseased cell or a cell from a non-diseased tissue sample (e.g., a cell from a healthy individual, or multiple cells from a population of healthy individuals). Any difference in the epitranscriptomic RNA modification profile of the cell or the profile of interactions between RNA-binding proteins and RNAs in the cell (including of a single RNA or of multiple RNAs of interest, e.g., a specific disease signature) relative to one or more non-diseased cells may indicate that the subject has the disease or disorder. Epitranscriptomic RNA modification and/or interactions between RNA-binding proteins and RNAs in one or more non-diseased cells (e.g., normal cells) may be profiled alongside expression in a diseased cell as a control experiment. Epitranscriptomic RNA modification and/or interactions between RNA-binding proteins and RNAs in one or more non-diseased cells (e.g., normal cells) may have also been profiled previously, and the profile of a diseased cell may be compared to this reference data for a non-diseased cell.

In another aspect, the present disclosure provides methods of screening for an agent capable of modulating epitranscriptomic modification of one or more RNAs of interest or interactions between RNA-binding proteins and one or more RNAs of interest. For example, the methods for profiling epitranscriptomic RNA modifications or interactions between RNA-binding proteins and RNAs described herein may be performed in a cell in the presence of one or more candidate agents. The expression of various epitranscriptomically modified RNAs of interest in the cell and/or the profile of interactions between RNA-binding proteins and RNAs in the cell (e.g., a normal cell, or a diseased cell) can then be compared to the expression of the same modified RNAs of interest or RNAs bound by an RNA-binding protein in a cell that was not exposed to the one or more candidate agents. Any difference in the epitranscriptomic RNA modification profile or the profile of interactions between RNA-binding proteins and RNAs relative to the cell that was not exposed to the candidate agent(s) may indicate that epitranscriptomic modification of the one or more RNAs of interest is modulated by the candidate agent(s). In some embodiments, a particular signature (e.g., of multiple epitranscriptomically modified RNAs of interest, or interactions between an RNA-binding protein and multiple RNAs of interest) that is known to be associated with the treatment of a disease may be used to identify agents capable of modulating epitranscriptomic RNA modification and/or interactions between RNA-binding proteins and RNAs in a desired manner and thus treating a disease. The methods and systems described herein may also be used to identify drugs that have certain side effects, for example, by looking for specific epitranscriptomic RNA modification signatures or RNA-binding protein-RNA interaction signatures when one or more cells is treated with a candidate agent or known drug (or combinations of multiple candidate agents and/or known drugs, e.g., as provided in a screening library of compounds). The methods and systems described herein may also be used to identify research reagents or chemical probes that may be useful for studying the basic biology of epitranscriptomic modification or interactions between RNA-binding proteins and RNAs.

In another aspect, the present disclosure provides methods for treating a disease or disorder in a subject. For example, the methods and systems for profiling epitranscriptomic RNA modification or interactions between RNA-binding proteins and RNAs described herein may be performed in a cell from a sample taken from a subject (e.g., a subject who is thought to have or is at risk of having a disease or disorder). The epitranscriptomic RNA modification profile or profile of interactions with RNA-binding proteins of one or more RNAs of interest in the cell can then be compared to the epitranscriptomic RNA modification profile or profile of interactions with RNA-binding proteins of one or more RNAs of interest in a cell from a non-diseased tissue sample. A treatment for the disease or disorder (e.g., a pharmaceutical agent, surgery, radiation therapy, surgery, physical therapy, lifestyle changes, etc.) may then be administered to the subject if any difference in the epitranscriptomic RNA modification profile or the profile of interactions between RNA-binding proteins and RNAs relative to a non-diseased cell is observed. Epitranscriptomic RNA modification and/or interactions between RNA-binding proteins and RNAs in one or more non-diseased cells may be profiled alongside epitranscriptomic RNA modification in a diseased cell as a control experiment. Epitranscriptomic RNA modification and/or interactions between RNA-binding proteins and RNAs in one or more non-diseased cells (e.g., normal cells) may have also been profiled previously, and epitranscriptomic RNA modification and/or interactions between RNA-binding proteins and RNAs in a diseased cell may be compared to this reference data for a non-diseased cell.

In another aspect, the present disclosure provides pairs of probes comprising a first probe (also referred to herein as the “padlock” probe) and a second probe (also referred to herein as the “primer” probe), wherein:
i) the first probe comprises an oligonucleotide portion that is complementary to the second probe, an oligonucleotide portion that is complementary to an RNA of interest, and an oligonucleotide barcode sequence (e.g., a unique sequence used to identify each RNA of interest, for example, by SEDAL sequencing as discussed herein); and
ii) the second probe comprises a portion that recognizes an epitranscriptomic RNA modification and an oligonucleotide portion that is complementary to a portion of the first probe.

In another aspect, the present disclosure provides sets of probes comprising a first probe (i.e., the “padlock probe”), a second probe (i.e., the “splint probe”), and a third probe (i.e., the “primer probe”), wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an epitranscriptomic RNA modification and an oligonucleotide portion that is complementary to a portion of the first probe; and
iii) the third probe comprises an oligonucleotide portion that is complementary to the RNA of interest and an oligonucleotide portion that is complementary to a portion of the first probe.

In another aspect, the present disclosure provides sets of probes comprising a first probe (i.e., the “padlock probe”), a second probe (i.e., the “splint probe”), and a third probe (i.e., the “primer probe”), wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an RNA-binding protein and an oligonucleotide portion that is complementary to a portion of the first probe; and
iii) the third probe comprises an oligonucleotide portion that is complementary to the RNA of interest and an oligonucleotide portion that is complementary to a portion of the first probe.

In another aspect, the present disclosure provides kits (e.g., a kit comprising any of the pairs of probes or sets of probes disclosed herein). In some embodiments, the kit comprises multiple pairs of probes or sets of probes as described herein, each of which can be used to identify a specific epitranscriptomically modified RNA of interest or interaction between a particular RNA-binding protein and RNA of interest. In certain embodiments, the kit comprises more than 1, more than 2, more than 3, more than 4, more than 5, more than 10, more than 20, more than 30, more than 40, more than 50, more than 100, more than 200, more than 500, more than 1000, more than 2000, or more than 3000 pairs of probes. In certain embodiments, the kit comprises more than 1, more than 2, more than 3, more than 4, more than 5, more than 10, more than 20, more than 30, more than 40, more than 50, more than 100, more than 200, more than 500, more than 1000, more than 2000, or more than 3000 sets of probes. The kits described herein may also include any other reagents or components useful in performing the methods described herein, including, but not limited to, cells, enzymes such as a ligase and/or a polymerase, amine-modified nucleotides, agents that bind an RNA modification (e.g., primary antibodies, secondary antibodies, proteins, peptides, aptamers, small molecules, etc.) buffers, reagents (including dyes, stains, buffers, and more), and monomers for making a polymeric matrix (e.g., a polyacrylamide matrix).

In another aspect, the present disclosure provides systems for profiling epitranscriptomic RNA modifications in a cell. In some embodiments, such a system comprises:
a) a cell (e.g., an isolated cell, or a cell present within an intact tissue);
b) one or more pairs of probes comprising a first probe (i.e., the “padlock probe”) and a second probe (i.e., the “primer probe”), wherein:
   i) the first probe comprises an oligonucleotide portion that is complementary to the second probe, an oligonucleotide portion that is complementary to an RNA of interest, and an oligonucleotide barcode sequence (e.g., a unique sequence used to identify each RNA of interest, for example, by SEDAL sequencing as discussed herein); and
   ii) the second probe comprises a portion that recognizes an epitranscriptomic RNA modification and an oligonucleotide portion that is complementary to a portion of the first probe;
c) a microscope; and
d) a computer.

In some embodiments, the present disclosure provides systems comprising:
a) a cell;
b) one or more sets of probes comprising a first probe, a second probe, and a third probe, wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an epitranscriptomic RNA modification and an oligonucleotide portion that is complementary to a portion of the first probe; and
   iii) the third probe comprises an oligonucleotide portion that is complementary to the RNA of interest and an oligonucleotide portion that is complementary to a portion of the first probe;
c) a microscope; and
d) a computer.

In some embodiments, the present disclosure provides systems comprising:
a) a cell;
b) one or more sets of probes comprising a first probe, a second probe, and a third probe, wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an RNA-binding protein and an oligonucleotide portion that is complementary to a portion of the first probe; and
   iii) the third probe comprises an oligonucleotide portion that is complementary to the RNA of interest and an oligonucleotide portion that is complementary to a portion of the first probe;
c) a microscope; and
d) a computer.

Any of the probes (i.e., the pairs of probes and sets of probes) described herein may be used in the systems contemplated by the present disclosure.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the Detailed Description of Specific Embodiments presented herein.

FIG. 1 provides an outline of a method for profiling of m⁶A-modified RNAs in situ. A secondary anti-m⁶A antibody is conjugated to a polymerizable DNA primer, which anneals to a padlock probe that resides on the same RNA. Functionalized cDNA amplicons are then generated from the co-localized DNA primer and padlock probes. Chemically functionalized cDNA amplicons are then covalently linked to a polyacrylamide matrix to allow tissue optical clearing and biomolecule processing.

FIGS. 2A-2D show an example of single-cell in situ profiling of m⁶A RNA modification. FIG. 2A shows detection of m⁶A-modified beta-actin RNAs. FIGS. 2B-2C provide several negative controls, i.e., no primary antibody (FIG. 2B), secondary antibody incubation with no DNA conjugated (FIG. 2C), and no secondary antibody (FIG. 2D).

FIGS. 3A-3D are images showing methods for resolving RNA modification mediated gene regulation in biological tissues by enabling single-cell in situ epitranscriptomic profiling. FIG. 3A shows various chemical modifications on eukaryotic messenger RNA. The collection of all the modification sites is termed the epitranscriptome. FIG. 3B provides a schematic showing single-cell heterogeneity of epitranscriptomic states across different cell types, states, and subcellular locations. These questions have not been addressed by previously developed bulk transcriptomic or epitranscriptomic analysis methods. FIG. 3C shows three-dimensional (3D) in situ sequencing of RNAs and RNA modifications. DNA-conjugated modification-specific binders are used together with RNA-sequence-specific DNA probes that hybridize to cellular mRNAs to selectively visualize modified RNAs in intact tissue. Each RNA-sequence-specific probe contains a barcode encoding identity of the gene, which is read-out through image-based in situ sequencing. Such highly multiplexed single-cell quantification of RNAs with chemical modification status in 3D enables single-cell discovery of cell types and epitranscriptomic cell states. FIG. 3D shows resolving RNA modification-mediated gene regulation in tissue as exemplified by the four possible changes of RNA modifications during brain activity. The shift of epitranscriptomic patterns, along with gene expression changes of RNA-modifying pathways, provides information on possible gene regulation schemes in different cells and brain regions. In situ epitranscriptomic sequencing can also be combined with precise genetic perturbation to fully dissect gene regulation mechanisms.

FIG. 4 provides a schematic showing questions regarding RNA modifications that cannot be anwsered by bulk epitranscriptomic sequencing methods.

FIG. 5 shows examples of mutagenic and non-mutagenic epitranscriptomic RNA modifications.

FIG. 6 provides a schematic showing the role of m⁶A in brain function. m⁶A modification is reversible and recognized by m⁶A-binding proteins, which regulate multiple steps of the mRNA life cycle. m⁶A-protein complexes have been found to be enriched in synapses. Alteration of the m⁶A pathway impacts multiple physiological functions and is associated with a spectrum of psychiatric disorders.

FIG. 7 provides a schematic of the 3D-m⁶A-seq method. After brain tissue is prepared, DNA-conjugated m⁶A-specific binders together with RNA-sequence-specific DNA probes will hybridize to cellular mRNAs within the intact tissue. Only m⁶A-modified sites are enzymatically replicated as cDNA amplicons, while non-modified RNAs are not amplified. Each RNA-sequence-specific probe contains a barcode encoding the identity of the gene and the m⁶A site, which is read-out through in situ sequencing (SEDAL).

FIG. 8 provides schematics of four exemplary biological investigations that may be carried out using single-cell in situ sequencing of RNA modifications. (1) Use of 3D-m⁶A-seq mapping of the cortex to determine whether the m⁶A methylome is cell-type and region specific; (2) use of 3D-m⁶A-seq to study global stimulation of neuronal cultures (KCI depolarization) or mouse visual cortex (dark/light conditioning) to determine whether activity-regulated genes (ARG) are differentially expressed in different cell types and if the m⁶A status co-varies with ARG; (3) use of 3D-m6A-seq to study m⁶A dynamics during specific experiences (e.g., acute restraint stress) to determine whether m⁶A has circuit preferences and varied responses in different brain regions; and (4) integrative analysis of single-cell m⁶A patterns and gene expression levels of m⁶A pathway proteins (e.g., methyltransferase, demethylase, and binding proteins) to determine which factors have shaped epitranscriptomic patterns and regulated gene expression in different cell types, and whether m⁶A-dependent gene regulation during neural stimulation is universal for every neuron or is more active (or more silent) in defined neuronal cell types of specific neural circuits in comparison to the average activity in the brain.

FIGS. 9A-9D show the background and applications of spatial epitranscriptomics.

FIG. 9A shows that m⁶A and its associated RNA-binding proteins (RBPs) regulate various important pathways. FIGS. 9B and 9C show knowledge gaps in epitranscriptomics that the methods provided herein are applicable to elucidating. FIG. 9D provides a schematic of in situ single-cell epitranscriptomics.

FIGS. 10A-10E show the design and principles of m⁶A-map v1. FIG. 10A shows the workflow of m⁶A-map v1. FIG. 10B shows the conjugation strategy used to synthesize the PAPG-oligo. FIG. 10C demonstrates the detection of actin β (ACTB) site 1217 m⁶A in HeLa cells via antibody-PAPG-oligo detection, showing a 20-30 fold signal enrichment over negative control. FIG. 10D demonstrates the detection of metastasis associated lung adenocarcinoma transcript 1 (MALAT1) site 2601 m⁶A in Hela cells via antibody-independent biotinylated-YTH-streptavidin-oligo detection, showing no enrichment over negative control. FIG. 10E demonstrates the detection of MALAT1 site 1248 m⁶A in mouse hippocampus via antibody-PAPG-oligo detection, showing an approximately 15-fold signal enrichment over negative control.

FIGS. 11A-11B show substitution of a secondary antibody for PAPG. FIG. 11A shows the labeling chemistry used by SiteClick antibody labeling kit (subsequent conjugation with alkyne-oligo can be performed using click chemistry). FIG. 11B demonstrates the detection of ACTB site 1217 m⁶A in Hela cells via antibody-PAPG-oligo detection and antibody-antibody-oligo detection. The antibody-antibody-oligo detection scheme showed much lower signal enrichment over IgG control groups.

FIGS. 12A-12D show the design and principles of m⁶A-map v2. FIG. 12A shows the workflow of m⁶A-map v2, achieving simultaneous detection of m⁶A-methylated RNAs and their non-methylated counterparts. FIG. 12B demonstrates the “winner-takes-all” behavior of the probes targeting the same RNA: when two pairs of STARmap probes (see PCT publication number WO 2019/199579, which is incorporated herein by reference) target the same ACTB mRNA in Hela cells, only one of them will get amplified at each ACTB mRNA molecule. FIG. 12C demonstrates the detection of MALAT1 site 2601 m⁶A in Hela cells via m⁶A-map v2, showing higher m6A stoichiometry in M and early G1 phases. FIG. 12D demonstrates the detection of ACTB site 1217 m⁶A in Hela cells via m⁶A-map v2, showing higher m6A stoichiometry in non-dividing cells.

FIGS. 13A-13C show RBP-map principles and experimental results. FIG. 13A shows the workflow for single RBP-RNA interaction mapping. FIG. 13B shows that HeLa cells transfected with 3xFLAG-YTH(WT/mut)-T2A-mCherry were tested for YTH binding of ACTB via the workflow shown in FIG. 13A. FIG. 13C shows the workflow for multiplexed RBP-RNA mapping. Each antibody is conjugated to a unique DNA primer with a corresponding gap-filling probe annealed to it. First, the three-part probes are hybridized to mRNAs. A mixture of different antibodies targeting different RBPs are then added to the sample, followed by ligation and RCA. Barcode information on the probe can be read out through in situ sequencing.

FIG. 14 provides representative images of the first round of sequencing in an m⁶A-map 100-gene data collection experiment. Ch01 and ch04 correspond to STARmap amplicons, while ch02 and ch03 correspond to m⁶A amplicons.

FIGS. 15A-15H show quality control and overview of statistics of m⁶A-map v1 100-gene dataset. Well labels: A1:STARmap; A2-anti-m⁶A: m⁶A-map v1 using Abcam ab151230; A3-anti-m⁶A: m⁶A-map v1 using SYSY 202003; B3-anti-m⁶A: m⁶A-map v1 using Invitrogen RM362; B2-anti-m⁶A: negative control of m⁶A-map v1 using CST 2729S normal rabbit IgG. FIG. 15A shows the average number of reads per cell for each gene. FIG. 15B shows the signal-to-noise ratio (calculated using the ratio of anti-m⁶A signal vs. IgG) in each well. FIG. 15C shows the detected genes and reads per cell in each well. FIG. 15D shows the correlation of estimated relative m⁶A stoichiometry between well A2 and well B3. FIG. 15E shows the correlation between estimated m⁶A stoichiometry and STARmap reads. FIG. 15F shows the relative m⁶A stoichiometry for each gene measured in each well. FIG. 15G provides a comparison of estimated m⁶A stoichiometry by m⁶A-map v1 vs. reported m⁶A stoichiometry of representative loci. FIG. 15H provides a scatter plot of the data shown in FIG. 15G.

FIGS. 16A-16C show subcellular analysis and cell cycle analysis of m⁶A-map v1 100-gene dataset. FIG. 16A shows the effect of m6A deposition on RNA subcellular localization (x-axis: the log₂ fold-change of nuclear percentage of m⁶A deposited portion vs. the non-m⁶A deposited portion of a given RNA; y-axis: −log₁₀ of p value). FIG. 16B shows that the cell cycle was determined using FUCCI fluorescent intensity. FIG. 16C shows representative genes with cell-cycle dependent m⁶A fluctuations.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The terms “administer,” “administering,” and “administration” refer to implanting, absorbing, ingesting, injecting, inhaling, or otherwise introducing a treatment or therapeutic agent, or a composition of treatments or therapeutic agents, in or on a subject.

The term “amplicon” as used herein refers to a nucleic acid (e.g., RNA) that is the product of an amplification reaction (i.e., the production of one or more copies of a genetic fragment or target sequence) or replication reaction. Amplicons can be formed artificially using, for example, PCR or other polymerization reactions. The term “concatenated amplicons” refers to multiple amplicons that are joined together to form a single nucleic acid molecule. Concatenated amplicons can be formed, for example, by rolling circle amplification (RCA), in which a circular oligonucleotide is amplified to produce multiple linear copies of the oligonucleotide as a single nucleic acid molecule comprising multiple amplicons that are concatenated.

An “antibody” refers to a glycoprotein belonging to the immunoglobulin superfamily. The terms antibody and immunoglobulin are used interchangeably. With some exceptions, mammalian antibodies are typically made of basic structural units each with two large heavy chains and two small light chains. There are several different types of antibody heavy chains, and several different kinds of antibodies, which are grouped together into different isotypes based on which heavy chain they possess. Five different antibody isotypes are known in mammals (IgG, IgA, IgE, IgD, and IgM), which perform different roles, and help direct the appropriate immune response for each different type of foreign object they encounter. The term “antibody” as used herein also encompasses antibody fragments, nanobodies, and single chain antibodies, as well as variants of antibodies. The term “antibody variants” may also be used to encompass antibody fragments. In some embodiments, an antibody or antibody variant is administered as a treatment for a disease or disorder (e.g., one that is associated with a change in the profile of epitranscriptomic RNA modifications in a cell taken from a subject). In some embodiments, an antibody is conjugated to an oligonucleotide probe as described herein. In certain embodiments, the antibody binds to an epitranscriptomic RNA modification (e.g., the antibody is an anti-m⁶A antibody, an anti-m¹A antibody, an anti-pseudouridine antibody, an anti-m⁶Am antibody, an anti-m⁷G antibody, an anti-ac⁴C antibody, an anti-Nm antibody, or an anti-m⁵C antibody).

A “cell,” as used herein, may be present in a population of cells (e.g., in a tissue, a sample, a biopsy, an organ, or an organoid). In some embodiments, a population of cells is composed of a plurality of different cell types. Cells for use in the methods and systems of the present disclosure can be present within an organism, a single cell type derived from an organism, or a mixture of cell types. Included are naturally occurring cells and cell populations, genetically engineered cell lines, cells derived from transgenic animals, cells from a subject, etc. Virtually any cell type and size can be accommodated in the methods and systems described herein. In some embodiments, the cells are mammalian cells (e.g., complex cell populations such as naturally occurring tissues). In some embodiments, the cells are from a human. In certain embodiments, the cells are collected from a subject (e.g., a human) through a medical procedure, such as a biopsy. Alternatively, the cells may be a cultured population (e.g., a culture derived from a complex population or a culture derived from a single cell type where the cells have differentiated into multiple lineages). The cells may also be provided in situ in a tissue sample.

Cell types contemplated for use in the methods and systems of the present disclosure include, but are not limited to, stem and progenitor cells (e.g., embryonic stem cells, hematopoietic stem cells, mesenchymal stem cells, neural crest cells, etc.), endothelial cells, muscle cells, myocardial cells, smooth and skeletal muscle cells, mesenchymal cells, epithelial cells, hematopoietic cells, lymphocytes such as T-cells (e.g., Th1 T cells, Th2 T cells, ThO T cells, cytotoxic T cells) and B cells (e.g., pre-B cells), monocytes, dendritic cells, neutrophils, macrophages, natural killer cells, mast cells, adipocytes, immune cells, neurons, hepatocytes, and cells involved with particular organs (e.g., thymus, endocrine glands, pancreas, brain, neurons, glia, astrocytes, dendrocytes, and genetically modified cells thereof). The cells may also be transformed or neoplastic cells of different types (e.g., carcinomas of different cell origins, lymphomas of different cell types, etc.) or cancerous cells of any kind (e.g., from any of the cancers disclosed herein). Cells of different origins (e.g., ectodermal, mesodermal, and endodermal) are also contemplated for use in the methods and systems of the present disclosure. In some embodiments, the cells are microglia, astrocytes, oligodendrocytes, excitatory neurons, or inhibitory neurons. In certain embodiments, the cells are Hela cells. In some embodiments, cells of multiple cell types are present within the same sample.

The term “complementary” is used herein to refer to two oligonucleotide sequences (e.g., DNA or RNA) comprising bases that hydrogen bond to one another. The degree of complementarity between two oligonucleotide sequences can vary, from complete complementarity to no complementarity (e.g., 100% complementarity, 99% complementarity, 98% complementarity, 97% complementarity, 96% complementarity, 95% complementarity, 90% complementarity, 85% complementarity, 80% complementarity, or less than 80% complementarity). For example, two oligonucleotide sequences may be only partially complementary to one another (e.g., in the probes described herein, wherein only a portion of the probe is complementary to another probe, or to an RNA of interest). In some embodiments, a sequence is complementary to only a portion of another sequence. In some embodiments, a sequence is complementary to another sequence under certain conditions (e.g., certain salt concentrations, pHs, etc.).

The terms “epitranscriptomic modification,” “epitranscriptomic RNA modification,” and “post-transcriptional modification” are used interchangeably throughout the present disclosure. Epitranscriptomic modifications include any biochemical modification of an RNA within a cell. Such modifications can be chemical modifications, including, for example, methylation of a nucleotide at various positions. Chemical epitranscriptomic modifications include, but are not limited to, N⁶-methyladenosine (m⁶A), N¹-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), and 5-methylcytosine (m⁵C). In certain embodiments, an epitranscriptomic modification is N⁶-methyladenosine (m⁶A). Other chemical epitranscriptomic RNA modifications include adenosine-to-inosine mutations and queuosine (i.e., queuosine replaces another nucleotide in the RNA). Various types of cellular RNA may be epitranscriptomically modified, including, but not limited to, ribosomal RNA (rRNA), transfer RNA (tRNA), and messenger RNA (mRNA). Other epitranscriptomic modifications include those described in Kumar, S. et al., Frontiers in Cell and Developmental Biology. 9 (2021); and Harcourt, E. M. et al. Nature. 541, 339-346 (2017).

The terms “polynucleotide,” “nucleotide sequence,” “nucleic acid,” “nucleic acid molecule,” “nucleic acid sequence,” and “oligonucleotide” refer to a series of nucleotide bases (also called “nucleotides”) in DNA and RNA and mean any chain of two or more nucleotides. The polynucleotides can be chimeric mixtures or derivatives or modified versions thereof, and single-stranded or double-stranded. The oligonucleotide can be modified at the base moiety, sugar moiety, or phosphate backbone, for example, to improve stability of the molecule, its hybridization parameters, etc. An “aptamer” is a type of oligonucleotide molecule that binds to a specific target molecule (e.g., an epitranscriptomic modification).

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these. A protein may also be a therapeutic protein administered as a treatment for a disease or disorder (e.g., one that is associated with a change in the profile of epitranscriptomic RNA modification in a cell taken from a subject). In certain embodiments, the protein is an antibody, or an antibody variant (including antibody fragments). In some embodiments, a protein binds to an epitranscriptomic RNA modification (e.g., an m⁶A-specific YTH domain protein).

An “RNA-binding protein” refers to any protein that is capable of binding RNA, or an epitranscriptomic modification of an RNA. For example, RNA-binding proteins may be proteins that introduce an epitranscriptomic modification onto an RNA. RNA-binding proteins may also be proteins that recognize and bind to an epitranscriptomic modification of an RNA. In certain embodiments, the RNA-binding protein comprises a YTH family protein (e.g., YTHDF1, YTHDF2, YTHDF3, YTHDC1, or YTHDC2), an IGF2BP family protein (e.g., IGF2BP1, IGF2BP2, or IGF2BP3), or FMR1. Additional RNA-binding proteins that can be profiled using the methods provided herein also include, but are not limited to, those disclosed in Wang, X. et al., N(6)-methyladenosine Modulates Messenger RNA Translation Efficiency. Cell 161, 1388-1399, doi:10.1016/j.cell.2015.05.014 (2015); Wang, X. et al., N6-methyladenosine-dependent regulation of messenger RNA stability. Nature 505, 117-120, doi:10.1038/nature12730 (2014); Shi, H. et al., YTHDF3 facilitates translation and decay of N(6)-methyladenosine-modified RNA. Cell Res 27, 315-328, doi:10.1038/cr.2017.15 (2017); Xiao, W. et al., Nuclear m(6)A Reader YTHDC1 Regulates mRNA Splicing. Mol Cell 61, 507-519, doi:10.1016/j.molcel.2016.01.012 (2016); Roundtree, I. A. et al., YTHDC 1 mediates nuclear export of N(6)-methyladenosine methylated mRNAs. Elife 6, doi:10.7554/eLife.31311 (2017); Hsu, P. J. et al., Ythdc 2 is an N(6)-methyladenosine binding protein that regulates mammalian spermatogenesis. Cell Res 27, 1115-1127, doi:10.1038/cr.2017.99 (2017); Huang, H. et al., Recognition of RNA N(6)-methyladenosine by IGF2BP proteins enhances mRNA stability and translation. Nat Cell Biol 20, 285-295, doi:10.1038/s41556-018-0045-z (2018); and Edens, B. M. et al., FMRP Modulates Neural Differentiation through m(6)A-Dependent mRNA Nuclear Export. Cell Rep 28, 845-854 e845, doi:10.1016/j.celrep.2019.06.072 (2019).

A “transcript” or “RNA transcript” is the product resulting from RNA polymerase-catalyzed transcription of a DNA sequence. When the RNA transcript is a complimentary copy of the DNA sequence, it is referred to as the primary transcript, or it may be an RNA sequence derived from post-transcriptional processing of the primary transcript and is referred to as the mature RNA. “Messenger RNA (mRNA)” refers to the RNA that is without introns and can be translated into polypeptides by the cell.

The term “sample” or “biological sample” refers to any sample including tissue samples (such as tissue sections, surgical biopsies, and needle biopsies of a tissue); cell samples; or cell fractions, fragments, or organelles (such as obtained by lysing cells and separating the components thereof by centrifugation or otherwise). Other examples of biological samples include, but are not limited to, blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. In some embodiments, a biological sample is a surgical biopsy taken from a subject, for example, a biopsy of any of the tissues described herein. In certain embodiments, a biological sample is a tumor biopsy. In some embodiments, the sample is brain tissue. In some embodiments, the tissue is cardiac tissue. In some embodiments, the sample is epithelial tissue, connective tissue, muscular tissue, or nervous tissue. In some embodiments, the sample is tissue from the central nervous system (e.g., brain). In some embodiments, the cells used in the methods described herein come from such a sample or biological sample.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., primate (e.g., cynomolgus monkey or rhesus monkey) or mouse). The term “patient” refers to a subject in need of treatment of a disease. In some embodiments, the subject is human. In some embodiments, the patient is human. The human may be a male or female at any stage of development. A subject or patient “in need” of treatment of a disease or disorder includes, without limitation, those who exhibit any risk factors or symptoms of a disease or disorder. In some embodiments, a subject is a non-human experimental animal (e.g., a mouse, rat, dog, or non-human primate).

The term “therapeutic agent,” as used herein, refers to any agent that can be used to treat a disease or disorder, or reduce or alleviate the symptoms of a disease or disorder. In some embodiments, the therapeutic agent is a small molecule, a protein, a peptide, a nucleic acid, a lipid, or a carbohydrate. In some embodiments, the therapeutic agent is a known drug and/or an FDA-approved drug. In certain embodiments, the protein is an antibody. In certain embodiments, the protein is an antibody variant. In certain embodiments, the protein is a receptor, or a fragment or variant thereof. In certain embodiments, the protein is a cytokine. In certain embodiments, the nucleic acid is an mRNA, an antisense RNA, an miRNA, an siRNA, an RNA aptamer, a double stranded RNA (dsRNA), a short hairpin RNA (shRNA), or an antisense oligonucleotide (ASO).

A “therapeutically effective amount” of a treatment or therapeutic agent is an amount sufficient to provide a therapeutic benefit in the treatment of a condition or to delay or minimize one or more symptoms associated with the condition. A therapeutically effective amount of a treatment or therapeutic agent means an amount of the therapy, alone or in combination with other therapies, that provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

As used herein, a “tissue” is a group of cells and their extracellular matrix from the same origin. Together, the cells carry out a specific function. The association of multiple tissue types together forms an organ. The cells may be of different cell types. In some embodiments, a tissue is an epithelial tissue. Epithelial tissues are formed by cells that cover an organ surface (e.g., the surface of the skin, airways, soft organs, reproductive tract, and inner lining of the digestive tract). Epithelial tissues perform protective functions and are also involved in secretion, excretion, and absorption. Examples of epithelial tissues include, but are not limited to, simple squamous epithelium, stratified squamous epithelium, simple cuboidal epithelium, transitional epithelium, pseudostratified epithelium, columnar epithelium, and glandular epithelium. In some embodiments, a tissue is a connective tissue. Connective tissues are fibrous tissues made up of cells separated by non-living material (e.g., an extracellular matrix). Connective tissues provide shape to organs and hold organs in place. Connective tissues include fibrous connective tissue, skeletal connective tissue, and fluid connective tissue. Examples of connective tissues include, but are not limited to, blood, bone, tendon, ligament, adipose, and areolar tissues. In some embodiments, a tissue is a muscular tissue. Muscular tissue is an active contractile tissue formed from muscle cells. Muscle tissue functions to produce force and cause motion. Muscle tissue includes smooth muscle (e.g., as found in the inner linings of organs), skeletal muscle (e.g., as typically attached to bones), and cardiac muscle (e.g., as found in the heart, where it contracts to pump blood throughout an organism). In some embodiments, a tissue is a nervous tissue. Nervous tissue includes cells comprising the central nervous system and peripheral nervous system. Nervous tissue forms the brain, spinal cord, cranial nerves, and spinal nerves (e.g., motor neurons). In certain embodiments, a tissue is brain tissue.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed (e.g., prophylactically or upon suspicion or risk of disease). In other embodiments, treatment may be administered in the absence of signs or symptoms of the disease. For example, treatment may be administered to a susceptible subject prior to the onset of symptoms (e.g., in light of a history of symptoms in the subject, or family members of the subject). Treatment may also be continued after symptoms have resolved, for example, to delay or prevent recurrence. In some embodiments, treatment may be administered after using the methods disclosed herein and observing a change in the profile of epitranscriptomic RNA modifications in a cell or tissue in comparison to a healthy cell or tissue.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

The present disclosure provides methods, compositions, and systems for profiling epitranscriptomic RNA modifications in a cell, or in multiple cells (e.g., cells present within an intact tissue, or isolated cells) at single-cell resolution and at subcellular resolution. Also provided by the present disclosure are methods for profiling interactions between one or more RNAs of interest in a cell and an RNA-binding protein (e.g., a protein that introduces an epitranscriptomic modification of an RNA of interest). The present disclosure also provides methods for diagnosing a disease or disorder in a subject based on a profile of epitranscriptomic RNA modifications or a profile of interactions between an RNA binding protein and RNA(s) in a cell, including cells within an intact tissue. The present disclosure also provides methods for treating a disease or disorder in a subject in need thereof. Methods of screening for or testing a candidate agent capable of modulating epitranscriptomic modification of one or more RNAs or interactions between one or more RNA(s) and an RNA-binding protein are also provided by the present disclosure. Pairs of probes and sets of probes, which may be useful for performing the methods described herein, are also provided by the present disclosure, as well as kits comprising any of the probes described herein.

### Methods for Profiling Epitranscriptomic RNA Modifications or Interactions Between RNAs and RNA-Binding Proteins in a Cell

In one aspect, the present disclosure provides methods for profiling epitranscriptomic RNA modifications in a cell (or in multiple cells, e.g., in an intact tissue). In the methods disclosed herein, a cell may be contacted with one or more pairs of probes or sets of probes, which are described further herein and may be used to identify and locate RNAs of interest comprising at least one epitranscriptomic modification to produce one or more concatenated amplicons. The one or more concatenated amplicons may then be embedded in a polymeric matrix and sequenced to determine the identity of the transcripts and their location within the polymeric matrix (e.g., through SEDAL sequencing as described further herein). Using the locations of the modified transcripts of interest, epitranscriptomically-modified RNAs in a cell may be profiled.

In some embodiments, the present disclosure provides methods for profiling epitranscriptomic RNA modification in a cell comprising the steps of:
a) contacting the cell with one or more sets of probes, wherein each set of probes comprises a first probe (i.e., the “padlock probe”), a second probe (i.e., the “splint probe”), and a third probe (i.e., the “primer probe”) wherein:
   i) the first probe comprises an oligonucleotide portion that is complementary to a portion of the second probe, an oligonucleotide barcode sequence (e.g., a unique sequence used to identify each RNA of interest, for example, by SEDAL sequencing as discussed herein), an oligonucleotide portion that is complementary to an RNA of interest, and an oligonucleotide portion that is complementary to a portion of the third probe;
   ii) the second probe comprises a portion that recognizes an epitranscriptomic RNA modification and an oligonucleotide portion that is complementary to a portion of the first probe; and
   iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell.

In some embodiments, the present disclosure provides methods for profiling epitranscriptomic RNA modifications in a cell (or in multiple cells, e.g., in an intact tissue) comprising the steps of:
a) contacting the cell with one or more pairs of probes, wherein each pair of probes comprises a first probe (i.e., the “padlock probe”) and a second probe (i.e., the “primer probe”), wherein:
   i) the first probe comprises an oligonucleotide portion that is complementary to the second probe, an oligonucleotide portion that is complementary to an RNA of interest, and an oligonucleotide barcode sequence (e.g., a unique sequence used to identify each RNA of interest, for example, by SEDAL sequencing as discussed herein); and
   ii) the second probe comprises a portion that recognizes an epitranscriptomic RNA modification and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell.

Profiling of any epitranscriptomic RNA modification is contemplated by the present disclosure. In some embodiments, the epitranscriptomic RNA modification is N⁶-methyladenosine (m⁶A), N1-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), or 5-methylcytosine (m⁵C). Other epitranscriptomic modifications include those described in Kumar, S. et al., Frontiers in Cell and Developmental Biology. 9 (2021); and Harcourt, E. M. et al., Nature. 541, 339-346 (2017). In some embodiments, the epitranscriptomic RNA modification is an adenosine-to-inosine modification. In some embodiments, the epitranscriptomic modification is queuosine (i.e., queuosine replaces another nucleotide in the RNA), polyadenylation, intron splicing, or histone mRNA processing. In certain embodiments, the epitranscriptomic RNA modification is N⁶-methyladenosine (m⁶A). A single epitranscriptomic modification may be profiled in a cell using the methods disclosed herein, or multiple different epitranscriptomic modifications (e.g., two, three, four, five, or more) may be profiled in the cell simultaneously.

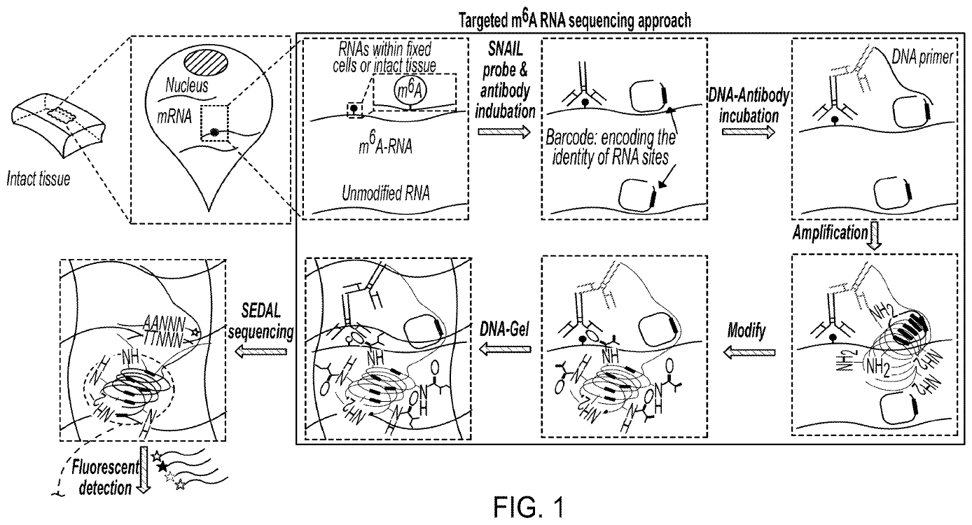

In some embodiments, the methods for profiling epitranscriptomic RNA modifications disclosed herein contemplate the use of a set of probes comprising a first probe, a second probe, and a third probe. Such methods utilize what is referred to herein as a “three-probe strategy.” The second probe of the set of probes (also referred to herein as the “splint probe”) comprises a portion that recognizes an epitranscriptomic RNA modification (i.e., a post-transcriptional modification present on a particular RNA of interest). In some embodiments, the portion of the second probe that binds the epitranscriptomic RNA modification comprises a peptide. In some embodiments, the portion of the second probe that binds the epitranscriptomic RNA modification comprises an aptamer. In some embodiments, the portion of the second probe that binds the epitranscriptomic RNA modification comprises a small molecule. In certain embodiments, the second probe binds to the epitranscriptomic modification through a mechanism comprising a biotin-streptavidin interaction. The portion of the probe that recognizes an epitranscriptomic RNA modification may be a protein (e.g., an antibody or antibody variant, or any protein that is otherwise capable of binding to a specific epitranscriptomic modification). In certain embodiments, the protein is PAPG. In some embodiments, the portion of the second probe that recognizes the epitranscriptomic RNA modification comprises an agent that binds an antibody, or an antibody variant. For example, the second probe may comprise a secondary antibody, and a primary antibody may be used to bind to the epitranscriptomic RNA modification. The secondary antibody on the second probe then recognizes the primary antibody bound to the epitranscriptomic RNA modification. See, for example, FIG. 1. In another example, the second probe may comprise the protein PAPG, which may bind an antibody that binds to the epitranscriptomic RNA modification. When the portion of the second probe that recognizes the epitranscriptomic RNA modification is a protein, the method may optionally further comprise contacting the cell with an antibody that recognizes an epitranscriptomic RNA modification, wherein the antibody that recognizes an epitranscriptomic RNA modification is recognized and bound by the protein of the second probe (e.g., PAPG). In certain embodiments, the portion of the second probe that recognizes the epitranscriptomic RNA modification comprises an antibody (e.g., a secondary antibody), or an antibody variant. When the portion of the second probe that recognizes the epitranscriptomic RNA modification is a secondary antibody, the method may optionally further comprise contacting the cell with a primary antibody that recognizes the epitranscriptomic RNA modification and is recognized by the secondary antibody of the second probe. The cell may be contacted with the primary antibody before or after being contacted with the one or more pairs of probes. In certain embodiments, the cell is contacted with the primary antibody prior to being contacted with the one or more pairs of probes. In some embodiments, the primary antibody is an anti-m6A antibody, an anti-m¹A antibody, an anti-pseudouridine antibody, an anti-m⁶Am antibody, an anti-m⁷G antibody, an anti-ac⁴C antibody, an anti-Nm antibody, or an anti-m⁵C antibody. In place of a primary antibody, the present disclosure also contemplates the use of any agent capable of binding an epitranscriptomic RNA modification directly on the probes described herein. In some embodiments, the portion of the second probe that binds the epitranscriptomic RNA modification directly comprises a protein. In certain embodiments, the protein is an m⁶A-specific YTH domain protein. In some embodiments, the portion of the second probe that binds the epitranscriptomic RNA modification directly comprises an antibody, or an antibody variant.

In some embodiments, the second probe of the set of probes further comprises a polymerization blocker. The polymerization blocker can be any moiety capable of preventing the use of the second oligonucleotide probe as a primer in the rolling circle amplification of step (c) of the methods described herein. In some embodiments, the polymerization blocker is at the 3′ end of the second oligonucleotide probe. The polymerization blocker can be, for example, any chemical moiety that prevents a polymerase from using the second oligonucleotide probe as a primer for polymerization. In some embodiments, the polymerization blocker is a nucleic acid residue comprising a blocked 3′ hydroxyl group (e.g., comprising an oxygen protecting group on the 3′ hydroxyl group). In some embodiments, the polymerization blocker comprises a hydrogen in place of the 3′ hydroxyl group. In some embodiments, the polymerization blocker comprises any chemical moiety in place of the 3′ hydroxyl group that prevents an additional nucleotide from being added. In some embodiments, the polymerization blocker comprises an inverted nucleic acid residue. In some embodiments, the polymerization blocker is an inverted adenosine, thymine, cytosine, guanosine, or uridine residue. In certain embodiments, the polymerization blocker is an inverted thymine residue.

In addition to the portion that recognizes the epitranscriptomic RNA modification, the second probe also comprises a portion that is complementary to a portion of the first probe. In the sets of probes provided herein, the portions of the first and second probes that are complementary to one another may be the same on each set of probes. In some embodiments, the portions of the first and second probes that are complementary to one another are unique on each of the first and second probes. In some embodiments, the portion of the second probe that is complementary to a portion of the first probe is about 3-20, about 4-19, about 5-18, about 6-17, about 7-16, about 8-15, about 9-14, about 10-13, or about 11-12 nucleotides in length. In some embodiments, the portion of the second probe that is complementary to a portion of the first probe is about 3, about 4, about 5, about 6, about 7, about 8, about 9, about 10, about 11, about 12, about 13, about 14, about 15, about 16, about 17, about 18, about 19, or about 20 nucleotides in length.

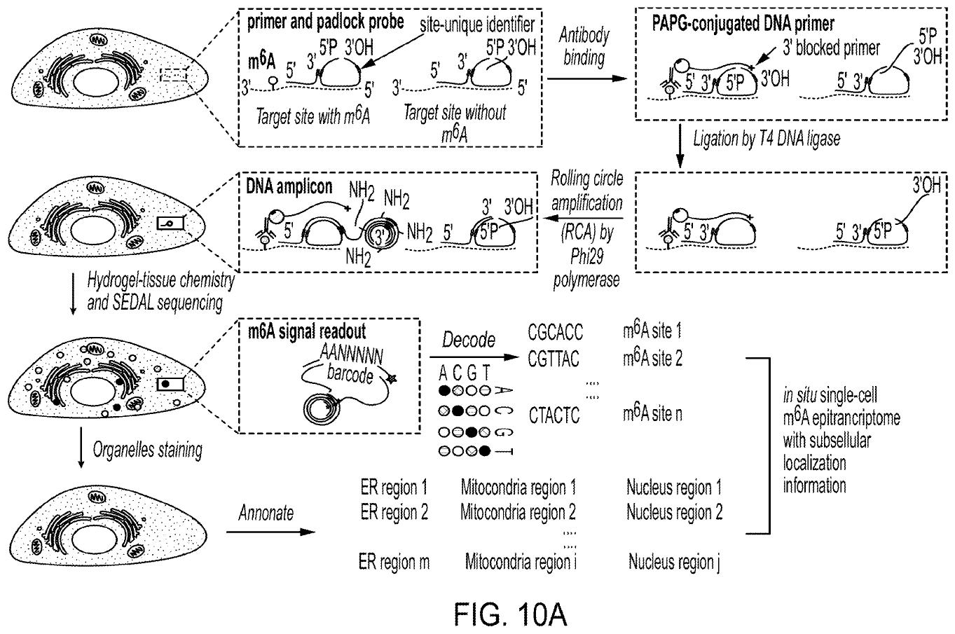

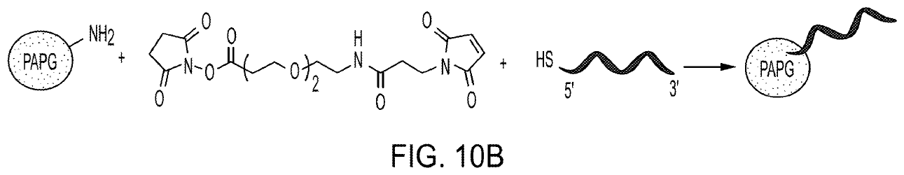

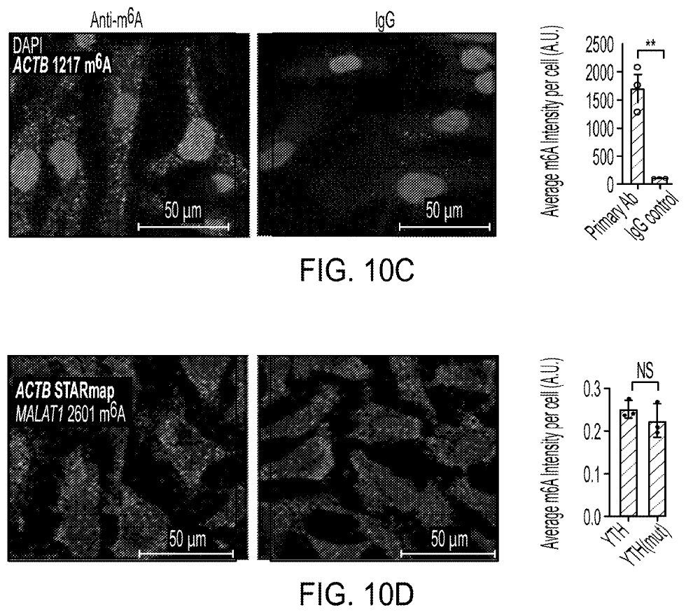

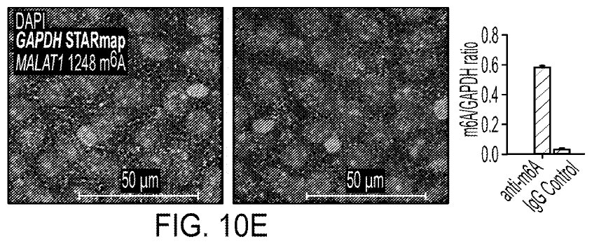

In some embodiments, each portion of the second probe is connected by an optional linker. In some embodiments, the optional linker is a nucleotide linker. In certain embodiments, the optional linker is 1, 2, 3, 4, 5, 6, 7, 8, 9, or 10 nucleotides long. In some embodiments, the second probe of the sets of probes used in the methods described herein comprises the structure:
5′-[portion recognizing epitranscriptomic RNA modification]-[portion complementary to first probe]-3′,
   wherein]-[comprises an optional linker (e.g., a nucleotide linker). In some embodiments, ]-[represents a direct linkage between two portions of the second probe (i.e., a phosphodiester bond). In some embodiments, the portion recognizing an epitranscriptomic RNA modification and the oligonucleotide portion of the probe are attached to each other via click chemistry (see, for example, FIG. 10B).

The first probe of the sets of probes used in the methods described herein (also referred to herein as the “padlock” probe) includes an oligonucleotide portion that is complementary to the second probe. In some embodiments, the portion of the first probe that is complementary to a portion of the second probe is about 4-20, about 5-19, about 6-18, about 7-17, about 8-16, about 9-15, about 10-14, or about 11-13 nucleotides in length. In some embodiments, the portion of the first probe that is complementary to a portion of the second probe is about 4, about 5, about 6, about 7, about 8, about 9, about 10, about 11, about 12, about 13, about 14, about 15, about 16, about 17, about 18, about 19, or about 20 nucleotides in length. In certain embodiments, the oligonucleotide portion of the first probe that is complementary to the second probe is split between the 5′ end and the 3′ end of the first probe.

The first probe of the sets of probes used in the methods disclosed herein also comprises an oligonucleotide portion that is complementary to an RNA of interest. In some embodiments, the oligonucleotide portion of the first probe that is complementary to the RNA of interest is about 10-30, about 11-29, about 12-28, about 13-27, about 14-26, about 15-25, about 16-24, about 17-23, about 18-22, or about 19-21 nucleotides in length. In some embodiments, the oligonucleotide portion of the first probe that is complementary to the RNA of interest is about 10, about 11, about 12, about 13, about 14, about 15, about 16, about 17, about 18, about 19, about 20, about 21, about 22, about 23, about 24, about 25, about 26, about 27, about 28, about 29, about 30, or more than 30 nucleotides in length.

The first probe of the sets of probes used in the methods disclosed herein also comprises an oligonucleotide barcode sequence made up of a specific sequence of nucleotides. In some embodiments, the oligonucleotide barcode sequence of the first probe is about 1-10, about 2-9, about 3-8, about 4-7, or about 5-6 nucleotides in length. In some embodiments, the oligonucleotide barcode sequence of the first probe is about 1, about 2,about 3, about 4, about 5, about 6, about 7, about 8, about 9, or about 10 nucleotides in length. The barcodes of the oligonucleotide probes described herein may comprise gene-specific sequences used to identify RNAs of interest (i.e., RNAs that have been modified with at least one particular epitranscriptomic modification).

The arrangement of the portions of the first oligonucleotide probe in any order is contemplated by the present disclosure. In some embodiments, a portion of the first probe is connected by an optional linker to another portion. In certain embodiments, the optional linker is 1, 2, 3, 4, 5, 6, 7, 8, 9, or 10 nucleotides long. In some embodiments, the first probe comprises the structure:
5′-[portion complementary to second probe]-[oligonucleotide barcode sequence]-[portion complementary to RNA of interest]-[portion complementary to third probe]-[portion complementary to second probe]-3′,
   wherein]-[comprises an optional nucleotide linker. In some embodiments,]-[represents a direct linkage between two portions of the first probe (i.e., a phosphodiester bond). In some embodiments, any of the oligonucleotide portions of the probes that make up the sets of probes provided herein comprise DNA.

The third probe of the sets of probes used in the methods provided herein (also referred to as the “primer probe”) comprises a portion complementary to an RNA of interest and a portion complementary to the first probe of the set of probes. In some embodiments, the portion of the third probe that is complementary to the RNA of interest is 10-30, 11-29, 12-28, 13-27, 14-26, 15-25, 16-24, 17-23, 18-22, or 19-21 nucleotides in length. In some embodiments, the portion of the third probe that is complementary to the RNA of interest is 10, 11, 12, 13, 14, 15, 16, 17, 18, 19, 20, 21, 22, 23, 24, 25, 26, 27, 28, 29, or 30 nucleotides in length. In some embodiments, the portion of the third probe that is complementary to a portion of the first probe is 5-15, 6-14, 7-13, 8-12, or 9-11 nucleotides in length. In some embodiments, the portion of the third probe that is complementary to a portion of the first probe is 5, 6, 7, 8, 9, 10, 11, 12, 13, 14, or 15 nucleotides in length.

In certain embodiments, the third probe of the set of probes comprises the structure:
5′-[portion complementary to RNA of interest]-[portion complementary to first probe]-3′, wherein]-[comprises an optional nucleotide linker. In some embodiments,]-[represents a direct linkage between two portions of the first probe (i.e., a phosphodiester bond).

In some embodiments, the methods disclosed for profiling epitranscriptomic RNA modifications herein contemplate the use of a pair of probes comprising a first probe and a second probe. Such methods utilize what is referred to herein as a “two-probe strategy.” The second probe of the pair of probes (also referred to herein as the “primer probe”) comprises a portion that recognizes an epitranscriptomic RNA modification (i.e., a post-transcriptional modification present on a particular RNA of interest). In some embodiments, the portion of the second probe that binds the epitranscriptomic RNA modification comprises a peptide. In some embodiments, the portion of the second probe that binds the epitranscriptomic RNA modification comprises an aptamer. In some embodiments, the portion of the second probe that binds the epitranscriptomic RNA modification comprises a small molecule. In certain embodiments, the second probe binds to the epitranscriptomic modification through a mechanism comprising a biotin-streptavidin interaction. The portion of the probe that recognizes an epitranscriptomic RNA modification may be a protein (e.g., an antibody or antibody variant, or any protein that is otherwise capable of binding to a specific epitranscriptomic modification). In some embodiments, the portion of the second probe that recognizes the epitranscriptomic RNA modification comprises an agent that binds an antibody, or an antibody variant. For example, the second probe may comprise a secondary antibody, and a primary antibody may be used to bind to the epitranscriptomic RNA modification. The secondary antibody on the second probe then recognizes the primary antibody bound to the epitranscriptomic RNA modification. See, for example, FIG. 1. In certain embodiments, the portion of the second probe that recognizes the epitranscriptomic RNA modification comprises an antibody (e.g., a secondary antibody), or an antibody variant. When the portion of the second probe that recognizes the epitranscriptomic RNA modification is a secondary antibody, the method may optionally further comprise contacting the cell with a primary antibody that recognizes the epitranscriptomic RNA modification and is recognized by the secondary antibody of the second probe. The cell may be contacted with the primary antibody before or after being contacted with the one or more pairs of probes. In certain embodiments, the cell is contacted with the primary antibody prior to being contacted with the one or more pairs of probes. In some embodiments, the primary antibody is an anti-m⁶A antibody, an anti-m¹A antibody, an anti-pseudouridine antibody, an anti-m⁶Am antibody, an anti-m⁷G antibody, an anti-ac⁴C antibody, an anti-Nm antibody, or an anti-m⁵C antibody. In place of an antibody, the present disclosure also contemplates the use of any agent capable of binding an epitranscriptomic RNA modification on the probes described herein. In some embodiments, the portion of the second probe that binds the epitranscriptomic RNA modification comprises a protein. In certain embodiments, the protein is PAPG. In some embodiments, when the portions of the second probe that recognizes the epitranscriptomic RNA modification comprises a protein, the method further comprises contacting the cell with an antibody that recognizes an epitranscriptomic RNA modification, wherein the antibody that recognizes an epitranscriptomic RNA modification is recognized and bound by PAPG. In certain embodiments, the protein is an m⁶A-specific YTH domain protein that can bind an epitranscriptomic RNA modification directly. In some embodiments, the portion of the second probe that binds the epitranscriptomic RNA modification binds the modification directly and comprises an antibody, or an antibody variant.

In addition to the portion that recognizes the epitranscriptomic RNA modification, the second probe of the pair of probes also comprises a portion that is complementary to a portion of the first probe. In the pairs of probes provided herein, the portions of the first and second probes that are complementary to one another may be the same on each set of probes. In some embodiments, the portions of the first and second probes that are complementary to one another are unique on each pair of probes. In some embodiments, the portion of the second probe that is complementary to a portion of the first probe is about 3-20, about 4-19, about 5-18, about 6-17, about 7-16, about 8-15, about 9-14, about 10-13, or about 11-12 nucleotides in length. In some embodiments, the portion of the second probe that is complementary to a portion of the first probe is about 3, about 4, about 5, about 6, about 7, about 8, about 9, about 10, about 11, about 12, about 13, about 14, about 15, about 16, about 17, about 18, about 19, or about 20 nucleotides in length.

In some embodiments, each portion of the second probe of the pair of probes is connected by an optional linker. In some embodiments, the optional linker is a nucleotide linker. In certain embodiments, the optional linker is 1, 2, 3, 4, 5, 6, 7, 8, 9, or 10 nucleotides long. In some embodiments, the second probe used in the methods described herein comprises the structure:
5′-[portion recognizing epitranscriptomic RNA modification]-[portion complementary to first probe]-3′,
   wherein]-[comprises an optional linker (e.g., a nucleotide linker). In some embodiments,]-[represents a direct linkage between two portions of the second probe (i.e., a phosphodiester bond).

The first probe of the pair of probes used in the methods described herein (also referred to herein as the “padlock” probe) includes an oligonucleotide portion that is complementary to the second probe. In some embodiments, the portion of the first probe that is complementary to a portion of the second probe is about 3-20, about 4-19, about 5-18, about 6-17, about 7-16, about 8-15, about 9-14, about 10-13, or about 11-12 nucleotides in length. In some embodiments, the portion of the first probe that is complementary to a portion of the second probe is about 3, about 4, about 5, about 6, about 7, about 8, about 9, about 10, about 11, about 12, about 13, about 14, about 15, about 16, about 17, about 18, about 19, or about 20 nucleotides in length. In certain embodiments, the oligonucleotide portion of the first probe that is complementary to the second probe is split between the 5′ end and the 3′ end of the first probe.

The first probe of the pair of probes used in the methods disclosed herein also comprises an oligonucleotide portion that is complementary to an RNA of interest. In some embodiments, the oligonucleotide portion of the first probe that is complementary to the RNA of interest is about 10-30, about 11-29, about 12-28, about 13-27, about 14-26, about 15-25, about 16-24, about 17-23, about 18-22, or about 19-21 nucleotides in length. In some embodiments, the oligonucleotide portion of the first probe that is complementary to the RNA of interest is about 10, about 11, about 12, about 13, about 14, about 15, about 16, about 17, about 18, about 19, about 20, about 21, about 22, about 23, about 24, about 25, about 26, about 27, about 28, about 29, about 30, or more than 30 nucleotides in length.

The first probe of the pair of probes used in the methods disclosed herein also comprises an oligonucleotide barcode sequence made up of a specific sequence of nucleotides. In some embodiments, the oligonucleotide barcode sequence of the first probe is about 1-10, about 2-9, about 3-8, about 4-7, or about 5-6 nucleotides in length. In some embodiments, the oligonucleotide barcode sequence of the first probe is about 1, about 2, about 3, about 4, about 5, about 6, about 7, about 8, about 9, or about 10 nucleotides in length. The barcodes of the oligonucleotide probes described herein may comprise gene-specific sequences used to identify RNAs of interest (i.e., RNAs that have been modified with at least one particular epitranscriptomic modification).

The arrangement of the portions of the first oligonucleotide probe of the pair of probes in any order is contemplated by the present disclosure. In some embodiments, a portion of the first probe is connected by an optional linker to another portion. In certain embodiments, the optional linker is 1, 2, 3, 4, 5, 6, 7, 8, 9, or 10 nucleotides long. In some embodiments, the first probe comprises the structure:
5′-[portion complementary to second probe]-[portion complementary to RNA of interest]-[barcode sequence]-[portion complementary to second probe]-3′;
5′-[portion complementary to second probe]-[barcode sequence]-[portion complementary to RNA of interest]-[portion complementary to second probe]-3′;
5′-[portion complementary to second probe]-[portion complementary to RNA of interest]-[barcode sequence]-3′;
5′-[portion complementary to second probe]-[barcode sequence]-[portion complementary to RNA of interest]-3′;
5′-[portion complementary to RNA of interest]-[barcode sequence]-[portion complementary to second probe]-3′; or
5′-[barcode sequence]-[portion complementary to RNA of interest]-[portion complementary to second probe]-3′,
   wherein]-[comprises an optional nucleotide linker. In some embodiments,]-[represents a direct linkage between two portions of the first probe (i.e., a phosphodiester bond). In some embodiments, any of the oligonucleotide portions of the first probe and/or the second probe comprise DNA.

In some embodiments, the present disclosure provides methods for profiling interactions between an RNA-binding protein and one or more RNAs of interest in a cell, the method comprising:
a) contacting the cell with one or more sets of probes, wherein each set of probes comprises a first probe, a second probe, and a third probe wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an RNA-binding protein and an oligonucleotide portion that is complementary to a portion of the first probe; and
   iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each RNA of interest bound by an RNA-binding protein in the cell.

The interaction of any RNA-binding protein with one or more RNAs of interest may be profiled using the methods provided herein. In some embodiments, the RNA-binding protein is a protein that introduces an epitranscriptomic modification onto an RNA. In certain embodiments, the RNA-binding protein comprises a YTH family protein (e.g., YTHDF1, YTHDF2, YTHDF3, YTHDC1, or YTHDC2), an IGF2BP family protein (e.g., IGF2BP1, IGF2BP2, or IGF2BP3), or FMR1. Additional RNA-binding proteins that can be profiled using the methods provided herein also include, but are not limited to, those disclosed in Wang, X. et al., N(6)-methyladenosine Modulates Messenger RNA Translation Efficiency. Cell 161, 1388-1399, doi:10.1016/j.cell.2015.05.014 (2015); Wang, X. et al., N6-methyladenosine-dependent regulation of messenger RNA stability. Nature 505, 117-120, doi:10.1038/nature12730 (2014); Shi, H. et al., YTHDF3 facilitates translation and decay of N(6)-methyladenosine-modified RNA. Cell Res 27, 315-328, doi:10.1038/cr.2017.15 (2017); Xiao, W. et al., Nuclear m(6)A Reader YTHDC1 Regulates mRNA Splicing. Mol Cell 61, 507-519, doi:10.1016/j. molcel.2016.01.012 (2016); Roundtree, I. A. et al., YTHDC1 mediates nuclear export of N(6)-methyladenosine methylated mRNAs. Elife 6, doi:10.7554/eLife.31311 (2017); Hsu, P. J. et al., Ythdc2 is an N(6)-methyladenosine binding protein that regulates mammalian spermatogenesis. Cell Res 27, 1115-1127, doi:10.1038/cr.2017.99 (2017); Huang, H. et al., Recognition of RNA N(6)-methyladenosine by IGF2BP proteins enhances mRNA stability and translation. Nat Cell Biol 20, 285-295, doi:10.1038/s41556-018-0045-z (2018); and Edens, B. M. et al., FMRP Modulates Neural Differentiation through m(6)A-Dependent mRNA Nuclear Export. Cell Rep 28, 845-854 e845, doi:10.1016/j.celrep.2019.06.072 (2019).

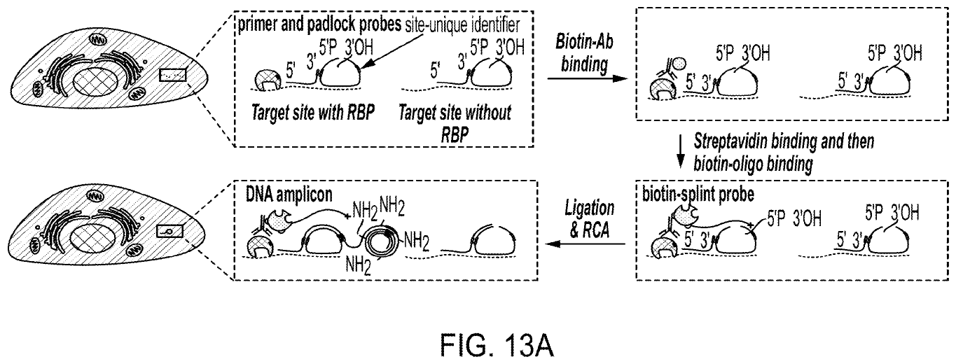

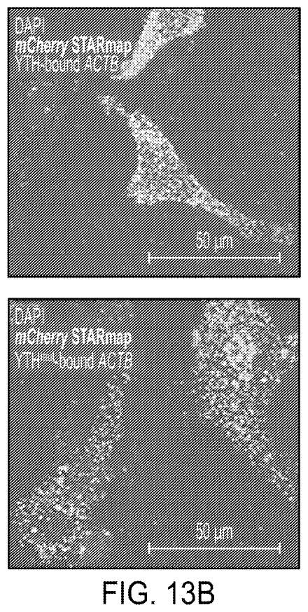

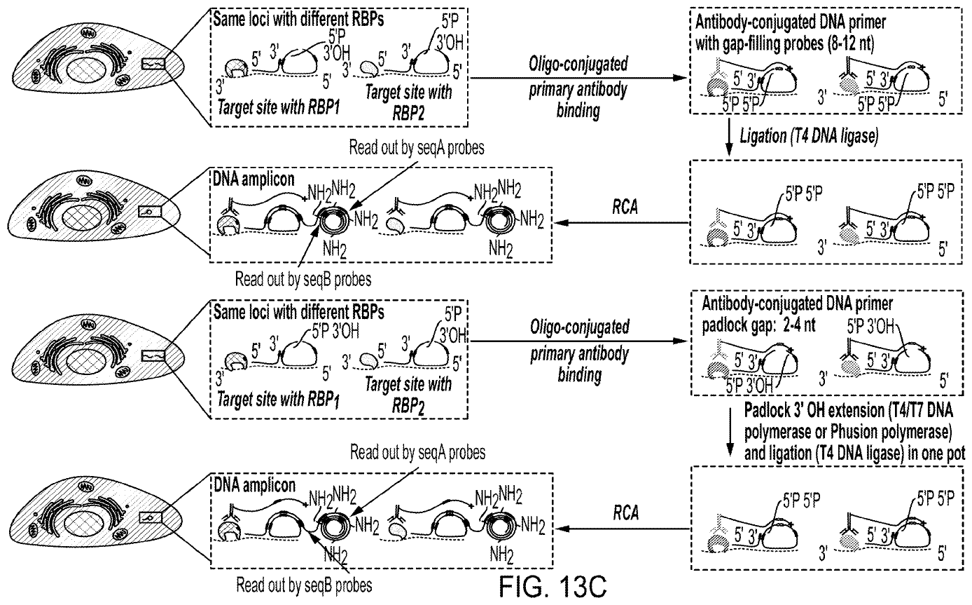

In some embodiments, the methods for profiling interactions between RNA-binding proteins and RNAs of interest disclosed herein contemplate the use of a set of probes comprising a first probe, a second probe, and a third probe. The second probe of the set of probes (also referred to herein as the “splint probe”) comprises a portion that recognizes an RNA-binding protein (e.g., an enzyme that introduces an epitranscriptomic modification onto an RNA). In some embodiments, the portion of the second probe that binds the RNA-binding protein comprises a peptide. In some embodiments, the portion of the second probe that binds the RNA-binding protein comprises an aptamer. In some embodiments, the portion of the second probe that binds the RNA-binding protein comprises a small molecule. In certain embodiments, the second probe binds to the RNA-binding protein through a mechanism comprising a biotin-streptavidin interaction. The portion of the probe that recognizes an RNA-binding protein may be a protein (e.g., an antibody or antibody variant, or any protein that is otherwise capable of binding to a specific RNA-binding protein). In some embodiments, the portion of the second probe that recognizes the RNA-binding protein comprises an agent that binds an antibody, or an antibody variant. For example, the second probe may comprise a secondary antibody, and a primary antibody may be used to bind to the RNA-binding protein. The secondary antibody on the second probe then recognizes the primary antibody bound to the RNA-binding protein. See, for example, FIGS. 13A and 13C. In certain embodiments, the portion of the second probe that recognizes the RNA-binding protein comprises an antibody (e.g., a secondary antibody), or an antibody variant. When the portion of the second probe that recognizes the RNA-binding protein is a secondary antibody, the method may optionally further comprise contacting the cell with a primary antibody that recognizes the RNA-binding protein and is recognized by the secondary antibody of the second probe. The cell may be contacted with the primary antibody before or after being contacted with the one or more pairs of probes. In certain embodiments, the cell is contacted with the primary antibody prior to being contacted with the one or more pairs of probes. In place of a primary antibody, the present disclosure also contemplates the use of any agent capable of recognizing and directly binding an RNA-binding protein on the probes described herein. In some embodiments, the portion of the second probe that binds the RNA-binding protein directly comprises a protein. In some embodiments, the portion of the second probe that binds the RNA-binding protein directly comprises an antibody, or an antibody variant.

In some embodiments, the second probe of the set of probes further comprises a polymerization blocker. The polymerization blocker can be any moiety capable of preventing the use of the second oligonucleotide probe as a primer in the rolling circle amplification of step (c) of the methods described herein. In some embodiments, the polymerization blocker is at the 3′ end of the second oligonucleotide probe. The polymerization blocker can be, for example, any chemical moiety that prevents a polymerase from using the second oligonucleotide probe as a primer for polymerization. In some embodiments, the polymerization blocker is a nucleic acid residue comprising a blocked 3′ hydroxyl group (e.g., comprising an oxygen protecting group on the 3′ hydroxyl group). In some embodiments, the polymerization blocker comprises a hydrogen in place of the 3′ hydroxyl group. In some embodiments, the polymerization blocker comprises any chemical moiety in place of the 3′ hydroxyl group that prevents an additional nucleotide from being added. In some embodiments, the polymerization blocker comprises an inverted nucleic acid residue. In some embodiments, the polymerization blocker is an inverted adenosine, thymine, cytosine, guanosine, or uridine residue. In certain embodiments, the polymerization blocker is an inverted thymine residue.

In addition to the portion that recognizes the RNA-binding protein, the second probe also comprises a portion that is complementary to a portion of the first probe. In the sets of probes provided herein, the portions of the first and second probes that are complementary to one another may be the same on each set of probes. In some embodiments, the portions of the first and second probes that are complementary to one another are unique on each of the first and second probes. In some embodiments, the portion of the second probe that is complementary to a portion of the first probe is about 3-20, about 4-19, about 5-18, about 6-17, about 7-16, about 8-15, about 9-14, about 10-13, or about 11-12 nucleotides in length. In some embodiments, the portion of the second probe that is complementary to a portion of the first probe is about 3, about 4, about 5, about 6, about 7, about 8, about 9, about 10, about 11, about 12, about 13, about 14, about 15, about 16, about 17, about 18, about 19, or about 20 nucleotides in length.

In some embodiments, each portion of the second probe is connected by an optional linker. In some embodiments, the optional linker is a nucleotide linker. In certain embodiments, the optional linker is 1, 2, 3, 4, 5, 6, 7, 8, 9, or 10 nucleotides long. In some embodiments, the second probe of the sets of probes used in the methods described herein comprises the structure:
5′-[portion recognizing RNA-binding protein]-[portion complementary to first probe]-3′,
   wherein ]-[comprises an optional linker (e.g., a nucleotide linker). In some embodiments, ]-[represents a direct linkage between two portions of the second probe (i.e., a phosphodiester bond).

The first probe of the sets of probes used in the methods described herein (also referred to herein as the “padlock” probe) includes an oligonucleotide portion that is complementary to the second probe. In some embodiments, the portion of the first probe that is complementary to a portion of the second probe is about 4-20, about 5-19, about 6-18, about 7-17, about 8-16, about 9-15, about 10-14, or about 11-13 nucleotides in length. In some embodiments, the portion of the first probe that is complementary to a portion of the second probe is about 4, about 5, about 6, about 7, about 8, about 9, about 10, about 11, about 12, about 13, about 14, about 15, about 16, about 17, about 18, about 19, or about 20 nucleotides in length. In certain embodiments, the oligonucleotide portion of the first probe that is complementary to the second probe is split between the 5′ end and the 3′ end of the first probe.

The first probe of the sets of probes used in the methods for profiling interactions between RNA-binding proteins and RNAs disclosed herein also comprises an oligonucleotide portion that is complementary to an RNA of interest. In some embodiments, the oligonucleotide portion of the first probe that is complementary to the RNA of interest is about 10-30, about 11-29, about 12-28, about 13-27, about 14-26, about 15-25, about 16-24, about 17-23, about 18-22, or about 19-21 nucleotides in length. In some embodiments, the oligonucleotide portion of the first probe that is complementary to the RNA of interest is about 10, about 11, about 12, about 13, about 14, about 15, about 16, about 17, about 18, about 19, about 20, about 21, about 22, about 23, about 24, about 25, about 26, about 27, about 28, about 29, about 30, or more than 30 nucleotides in length.

The first probe of the sets of probes used in the methods disclosed herein also comprises an oligonucleotide barcode sequence made up of a specific sequence of nucleotides. In some embodiments, the oligonucleotide barcode sequence of the first probe is about 1-10, about 2-9, about 3-8, about 4-7, or about 5-6 nucleotides in length. In some embodiments, the oligonucleotide barcode sequence of the first probe is about 1, about 2, about 3, about 4, about 5, about 6, about 7, about 8, about 9, or about 10 nucleotides in length. The barcodes of the oligonucleotide probes described herein may comprise gene-specific sequences used to identify RNAs of interest (i.e., RNAs that are bound by at least one RNA-binding protein).

The arrangement of the portions of the first oligonucleotide probe in any order is contemplated by the present disclosure. In some embodiments, a portion of the first probe is connected by an optional linker to another portion. In certain embodiments, the optional linker is 1, 2, 3, 4, 5, 6, 7, 8, 9, or 10 nucleotides long. In some embodiments, the first probe comprises the structure:
5′-[portion complementary to second probe]-[oligonucleotide barcode sequence]-[portion complementary to RNA of interest]-[portion complementary to third probe]-[portion complementary to second probe]-3′,
   wherein ]-[comprises an optional nucleotide linker. In some embodiments,]-[represents a direct linkage between two portions of the first probe (i.e., a phosphodiester bond). In some embodiments, any of the oligonucleotide portions of the probes that make up the sets of probes provided herein comprise DNA.

The third probe of the sets of probes used in the methods for profiling interactions between RNA-binding proteins and RNAs provided herein (also referred to as the “primer probe”) comprises a portion complementary to an RNA of interest and a portion complementary to the first probe of the set of probes. In some embodiments, the portion of the third probe that is complementary to the RNA of interest is 10-30, 11-29, 12-28, 13-27, 14-26, 15-25, 16-24, 17-23, 18-22, or 19-21 nucleotides in length. In some embodiments, the portion of the third probe that is complementary to the RNA of interest is 10, 11, 12, 13, 14, 15, 16, 17, 18, 19, 20, 21, 22, 23, 24, 25, 26, 27, 28, 29, or 30 nucleotides in length. In some embodiments, the portion of the third probe that is complementary to a portion of the first probe is 5-15, 6-14, 7-13, 8-12, or 9-11 nucleotides in length. In some embodiments, the portion of the third probe that is complementary to a portion of the first probe is 5, 6, 7, 8, 9, 10, 11, 12, 13, 14, or 15 nucleotides in length.

In certain embodiments, the third probe of the set of probes comprises the structure:
5′-[portion complementary to RNA of interest]-[portion complementary to first probe]-3′, wherein ]-[comprises an optional nucleotide linker. In some embodiments,]-[represents a direct linkage between two portions of the first probe (i.e., a phosphodiester bond).

The use of any type of cell in the methods disclosed herein is contemplated by the present disclosure (e.g., any of the cell types described herein). In some embodiments, the cell is a mammalian cell. In certain embodiments, the cell is a human cell. The present disclosure also contemplates performing the methods for profiling epitranscriptomic RNA modifications or interactions between RNA-binding proteins and RNAs described herein on multiple cells simultaneously. In some embodiments, the method is performed on multiple cells of the same cell type. In some embodiments, the method is performed on multiple cells comprising cells of different cell types. In some embodiments, epitranscriptomic RNA modifications or interactions between RNA-binding proteins and RNAs are profiled in more than 10 cells, more than 20 cells, more than 50 cells, more than 100 cells, more than 200 cells, more than 300 cells, more than 400 cells, more than 500 cells, or more than 1000 cells simultaneously. The cell types in which epitranscriptomic RNA modifications or interactions between RNA-binding proteins and RNAs may be profiled using the methods disclosed herein include, but are not limited to, stem cells, progenitor cells, neuronal cells, astrocytes, dendritic cells, endothelial cells, microglia, oligodendrocytes, muscle cells, myocardial cells, mesenchymal cells, epithelial cells, immune cells, hepatic cells, smooth and skeletal muscle cells, hematopoietic cells, lymphocytes, monocytes, neutrophils, macrophages, natural killer cells, mast cells, adipocytes, and neurons. In certain embodiments, the cell or cells are present within an intact tissue (e.g., of any of the tissue types described herein). In certain embodiments, the intact tissue is a fixed tissue sample. In some embodiments, the intact tissue comprises multiple cell types. In some embodiments, the tissue is epithelial tissue, connective tissue, muscular tissue, or nervous tissue. In certain embodiments, the tissue is cardiac tissue, lymph node tissue, liver tissue, muscle tissue, bone tissue, eye tissue, brain tissue, or ear tissue.

The RNAs of interest for which epitranscriptomic modification or interactions with RNA-binding proteins are profiled in the methods described herein may be transcripts that have been expressed from the genomic DNA of the cell. In some embodiments, the RNAs of interest are messenger RNA (mRNA), transfer RNA (tRNA), and/or ribosomal RNA (rRNA). In some embodiments, the RNAs of interest comprise transcripts that have not been processed yet (e.g., pre-mRNA). The methods described herein may be used to profile one epitranscriptomically-modified RNA or RNA bound by an RNA-binding protein in a cell at a time, or multiple epitranscriptomically-modified RNAs of interest or RNAs of interest bound by an RNA-binding protein simultaneously. In some embodiments, epitranscriptomic RNA modifications or interactions between RNA-binding proteins and RNAs in a cell, or in multiple cells, are profiled for more than 1, more than 2, more than 3, more than 4, more than 5, more than 10, more than 20, more than 30, more than 40, more than 50, more than 100, more than 200, more than 500, more than 1000, more than 2000, or more than 3000 RNAs simultaneously.

A polymeric matrix is used in the methods described herein following rolling circle amplification to facilitate sequencing and imaging of the epitranscriptomically-modified RNAs of interest or RNAs of interest bound by an RNA-binding protein in the cell. The use of various polymeric matrices is contemplated by the present disclosure, and any polymeric matrix in which the one or more concatenated amplicons can be embedded is suitable for use in the methods described herein. In some embodiments, the polymeric matrix is a hydrogel (i.e., a network of crosslinked polymers that are hydrophilic). In some embodiments, the hydrogel is a polyvinyl alcohol hydrogel, a polyethylene glycol hydrogel, a polyacrylate hydrogel, or a polyacrylamide hydrogel. In certain embodiments, the hydrogel is a polyacrylamide hydrogel. Such a hydrogel may be prepared, for example, by incubating the sample in a buffer comprising acrylamide and bis-acrylamide, removing the buffer, and incubating the sample in a polymerization mixture (comprising, e.g., ammonium persulfate and tetramethylethylenediamine). Such reagents may also be provided in a kit, e.g., a kit for performing any of the methods described herein, or any of the kits described herein.

In some embodiments, the step of performing rolling circle amplification to amplify the circular oligonucleotide to produce one or more concatenated amplicons further comprises providing nucleotides modified with reactive chemical groups (e.g., amine modified nucleotides such as 5-(3-aminoallyl)-dUTP). In some embodiments, the nucleotides modified with reactive chemical groups make up about 5%, about 6%, about 7%, about 8%, about 9%, or about 10% of the nucleotides used in the amplification reaction. For example, the step of performing rolling circle amplification to amplify the circular oligonucleotide to produce one or more concatenated amplicons may further comprise providing amine-modified nucleotides such as 5-(3-aminoallyl)-dUTP. During the amplification process, the amine-modified nucleotides are incorporated into the one or more concatenated amplicons as they are produced. The resulting amplicons are functionalized with primary amines, which can be further reacted with another compatible chemical moiety (e.g., N-hydroxysuccinimide) to facilitate the step of embedding the concatenated amplicons in the polymeric matrix. In some embodiments, the step of embedding the one or more concatenated amplicons in a polymeric matrix comprises reacting the amine-modified nucleotides of the one or more concatenated amplicons with acrylic acid N-hydroxysuccinimide ester and co-polymerizing the one or more concatenated amplicons and the polymer matrix.

The methods disclosed herein also include a step of sequencing the concatenated amplicons, or a portion thereof, embedded in the polymeric matrix. In some embodiments, the step of sequencing comprises performing “sequencing with error-reduction by dynamic annealing and ligation” (SEDAL sequencing). SEDAL sequencing is described further in Wang, X. et al., Three-dimensional intact-tissue sequencing of single-cell transcriptional states. Science 2018, 361, 380, and International Patent Application Publication No. WO 2019/199579, published Oct. 17, 2019, each of which is incorporated herein by reference. In brief, an oligonucleotide probe comprising a detectable label (i.e., any label that can be used to visualize the location of the additional oligonucleotide probe, for example, through imaging) is provided to the cell. In certain embodiments, the detectable label is fluorescent (e.g., a fluorophore). The additional oligonucleotide probe is complementary to the oligonucleotide barcode sequence of the first probe and is thus linked to the identity of an RNA of interest and can be used to identify the location of an RNA of interest within the cell (or within an organelle when the method is performed at subcellular resolution, e.g., with staining to identify the locations of individual organelles).

The additional oligonucleotide probe used in the methods described herein (e.g., as used in SEDAL sequencing) may be read out using any suitable imaging technique known in the art. For example, in embodiments where the additional oligonucleotide probe comprises a fluorophore, the fluorophore may be read out using imaging to identify the RNA of interest. As discussed above, the additional oligonucleotide probe comprises a sequence complementary to a barcode sequence on the first oligonucleotide probe, which is used to detect a specific RNA of interest. By imaging the location of the additional oligonucleotide probe comprising a fluorophore, the location of that specific RNA of interest within the sample can be determined. In some embodiments, the step of imaging comprises fluorescent imaging. In certain embodiments, the step of imaging comprises confocal microscopy. In certain embodiments, the step of imaging comprises epifluorescence microscopy. In certain embodiments, two rounds of imaging are performed. In certain embodiments, three rounds of imaging are performed. In certain embodiments, four rounds of imaging are performed. In certain embodiments, five or more rounds of imaging are performed.

In some embodiments, the methods for profiling epitranscriptomic RNA modifications or interactions between RNA-binding proteins and RNAs described herein may be combined with methods for profiling additional molecules within the cell. For example, expression of additional RNAs (including RNAs that have not been epitranscriptomically modified and RNAs that are not bound by an RNA-binding protein) may be profiled alongside epitranscriptomically modified RNAs or RNAs bound by one or more RNA-binding proteins using probes that do not comprise a portion that recognizes an epitranscriptomic RNA modification or RNA-binding protein. Methods for profiling other types of molecules (e.g., DNAs, proteins, carbohydrates, or lipids) may be combined with the methods described herein as well. In certain embodiments, the additional molecules which are profiled are unmodified RNAs. In some embodiments, profiling unmodified RNAs comprises:
a) contacting the cell with one or more pairs of probes, wherein each pair of probes comprises a first probe and a second probe, wherein:
   i) the first probe comprises an oligonucleotide portion complementary to a portion of the second probe, an oligonucleotide barcode sequence, and an oligonucleotide portion complementary to an unmodified RNA of interest; and
ii) the second probe comprises a portion that is complementary to the unmodified RNA of interest and a portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each unmodified RNA of interest in the cell.

In some embodiments, the methods provided herein further comprise determining the cell type of the profiled cell by comparing the epitranscriptomic RNA modification profile (or profile of interactions between RNAs and RNA-binding proteins) of a cell to reference data comprising epitranscriptomic RNA modification profiles (or profiles of interactions between RNAs and RNA-binding proteins) of various cell types. In some embodiments, the method further comprises overexpressing or knocking out one or more genes in the cell to determine whether the one or more genes are involved in epitranscriptomic modification of the RNA of interest. In some embodiments, the method further comprises repeating steps (a)-(e) at multiple time points to profile epitranscriptomic RNA modification or interactions between RNA-binding proteins and RNA in the cell over time. In some embodiments, the methods further comprise examining how the profile of epitranscriptomically modified RNAs or interactions between RNAs and RNA-binding proteins in a cell or multiple cells is affected by an immune response within an intact tissue, or how the profile is affected due to proximity to a tumor.

In some embodiments, any of the methods described herein further comprise resolving the locations of the modified RNAs of interest at subcellular resolution (i.e., within specific organelles within the cells; approximately 150-400 nm spatial resolution, depending on both the size of DNA amplicons and optical limits). This may be accomplished through organelle staining procedures, which are well known in the art. For example, various organelles such as the endoplasmic reticulum (ER), the cytoskeleton, and the mitochondria may be stained. In some embodiments, the agents used to stain various organelles in the cells comprise small molecule dyes, antibodies, and/or protein dyes.

### Methods for Diagnosing a Disease or Disorder in a Subject

In another aspect, the present disclosure provides methods for diagnosing a disease or disorder in a subject. For example, the methods for profiling epitranscriptomic RNA modifications or interactions between RNA-binding proteins and RNAs described herein may be performed on a cell or multiple cells (e.g., in an intact tissue) taken from a subject (e.g., a subject who is thought to have or is at risk of having a disease or disorder, or a subject who is healthy or thought to be healthy). The expression of various RNAs of interest in the cell can then be compared to the expression of the same RNAs of interest in a non-diseased cell or a cell from a non-diseased tissue sample (e.g., a cell from a healthy individual, or multiple cells from a population of healthy individuals). Any difference in the epitranscriptomic RNA modification profile or the profile of interactions between RNA-binding proteins and RNAs of the cell (including of a single RNA or of multiple RNAs of interest, e.g., a specific disease signature) relative to one or more non-diseased cells may indicate that the subject has the disease or disorder. Epitranscriptomic RNA modifications or interactions between RNA-binding proteins and RNAs in one or more non-diseased cells may be profiled alongside expression in a diseased cell as a control experiment. Epitranscriptomic RNA modifications or interactions between RNA-binding proteins and RNAs in one or more non-diseased cells may have also been profiled previously, and expression in a diseased cell may be compared to this reference data for a non-diseased cell.

In some embodiments, a method for diagnosing a disease or disorder in a subject comprises the steps of:
a) contacting a cell taken from a subject with one or more sets of probes, wherein each set of probes comprises a first probe, a second probe, and a third probe wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an epitranscriptomic RNA modification and an oligonucleotide portion that is complementary to a portion of the first probe; and
   iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell;

wherein a difference in the profile of epitranscriptomic RNA modifications in the cell relative to one or more non-diseased cells indicates that the subject has the disease or disorder.

In some embodiments, a method for diagnosing a disease or disorder in a subject comprises the steps of:
a) contacting a cell (or in multiple cells, e.g., in an intact tissue) taken from a subject with one or more pairs of probes, wherein each pair of probes comprises a first probe (i.e., the “padlock probe”) and a second probe (i.e., the “primer probe”), wherein:
   i) the first probe comprises an oligonucleotide portion that is complementary to the second probe, an oligonucleotide portion that is complementary to an RNA of interest, and an oligonucleotide barcode sequence (e.g., a unique sequence used to identify each RNA of interest, for example, by SEDAL sequencing as discussed herein); and
   ii) the second probe comprises a portion that recognizes an epitranscriptomic RNA modification and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell;
wherein a difference in the profile of epitranscriptomic RNA modifications in the cell relative to one or more non-diseased cells indicates that the subject has the disease or disorder.

In some embodiments, a method for diagnosing a disease or disorder in a subject comprises the steps of:
a) contacting a cell taken from a subject with one or more sets of probes, wherein each set of probes comprises a first probe, a second probe, and a third probe wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an RNA-binding protein and an oligonucleotide portion that is complementary to a portion of the first probe; and
   iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each RNA of interest bound to an RNA-binding protein in the cell;
wherein a difference in the profile of interactions between RNA and RNA-binding proteins in the cell relative to one or more non-diseased cells indicates that the subject has the disease or disorder.

In some embodiments, epitranscriptomic RNA modifications in one or more non-diseased cells, or the interactions between RNAs and RNA-binding proteins in one or more non-diseased cells, are profiled simultaneously alongside the cell taken from a subject using the methods disclosed herein as a control experiment. In some embodiments, the profile of epitranscriptomic RNA modifications in one or more non-diseased cells, or the profile of interactions between RNAs and RNA-binding proteins in one or more non-diseased cells, that is compared to expression in a diseased cell comprises reference data from when the method was performed on one or more non-diseased cells previously. Profiling of any epitranscriptomic RNA modification for diagnosis of a disease or disorder in a subject is contemplated by the present disclosure. In some embodiments, the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), or 5-methylcytosine (m⁵C). Other epitranscriptomic modifications include those described in Kumar, S. et al., Frontiers in Cell and Developmental Biology. 9 (2021); and Harcourt, E. M. et al., Nature. 541, 339-346 (2017). In some embodiments, the epitranscriptomic RNA modification is an adenosine-to-inosine modification. In some embodiments, the epitranscriptomic modification is queuosine (i.e., queuosine replaces another nucleotide in the RNA), polyadenylation, intron splicing, or histone mRNA processing. In certain embodiments, the epitranscriptomic RNA modification is N⁶-methyladenosine (m⁶A). A single epitranscriptomic modification may be profiled in a cell to diagnose a disease or disorder in a subject using the methods disclosed herein, or multiple different epitranscriptomic modifications (e.g., two, three, four, five, or more) may be profiled in the cell simultaneously. Profiling of interactions between RNAs and any RNA-binding protein is also contemplated by the present disclosure. In some embodiments, the RNA-binding protein is an enzyme that introduces an epitranscriptomic modification onto an RNA. In certain embodiments, the RNA-binding protein is a YTH family protein (e.g., YTHDF1, YTHDF2, YTHDF3, YTHDC1, or YTHDC2), an IGF2BP family protein (e.g., IGF2BP1, IGF2BP2, or IGF2BP3), or FMR1. Interactions between RNAs and a single RNA-binding protein may be profiled in a cell to diagnose a disease or disorder in a subject using the methods disclosed herein, or interactions with multiple different RNA-binding proteins (e.g., two, three, four, five, or more) may be profiled in the cell simultaneously.

Diagnosis of any disease or disorder is contemplated by the methods described herein. In some embodiments, the disease or disorder is a genetic disease, a proliferative disease, an inflammatory disease, an autoimmune disease, a liver disease, a spleen disease, a lung disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, a central nervous system (CNS) disorder, or a cardiovascular disease.

In some embodiments, the cell is present in a tissue (e.g., epithelial tissue, connective tissue, muscular tissue, or nervous tissue). In some embodiments, the tissue is a tissue sample from a subject. In some embodiments, the subject is a non-human experimental animal (e.g., a mouse). In some embodiments, the subject is a domesticated animal. In some embodiments, the subject is a human. In some embodiments, the tissue sample comprises a fixed tissue sample. In certain embodiments, the tissue sample is a biopsy (e.g., bone, bone marrow, breast, gastrointestinal tract, lung, liver, pancreas, prostate, brain, nerve, renal, endometrial, cervical, lymph node, muscle, or skin biopsy). In certain embodiments, the biopsy is a tumor biopsy. In certain embodiments, the tissue is brain tissue. In certain embodiments, the tissue is from the central nervous system.

### Methods for Treating a Disease or Disorder in a Subject

In another aspect, the present disclosure provides methods for treating a disease or disorder in a subject. For example, the methods for profiling epitranscriptomic RNA modification or interactions between RNA-binding proteins and RNAs described herein may be performed in a cell (or in multiple cells, e.g., in an intact tissue) from a sample taken from a subject (e.g., a subject who is thought to have or is at risk of having a disease or disorder). The profile of epitranscriptomic modifications of one or more RNAs or the profile of interactions between RNA-binding proteins and one or more RNAs in the cell can then be compared to the epitranscriptomic modification profile or the profile of interactions with RNA-binding proteins of the same RNAs of interest in a cell from a non-diseased tissue sample. A treatment for the disease or disorder may then be administered to the subject if any difference in the profile of epitranscriptomic RNA modifications or the profile of interactions between RNA-binding proteins and one or more RNAs in the cell relative to a non-diseased cell is observed. Epitranscriptomic RNA modification and/or interactions between RNA-binding proteins and RNAs in one or more non-diseased cells may be profiled alongside epitranscriptomic RNA modification in a diseased cell as a control experiment.

Epitranscriptomic RNA modification and/or interactions between RNA-binding proteins and RNAs in one or more non-diseased cells may have also been profiled previously, and the profile of epitranscriptomic RNA modification or interactions between RNA-binding proteins and RNAs in a diseased cell may be compared to this reference data for a non-diseased cell.

In some embodiments, the present disclosure provides methods for treating a disease or disorder in a subject comprising the steps of:
a) contacting a cell taken from a subject with one or more sets of probes, wherein each set of probes comprises a first probe, a second probe, and a third probe wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an epitranscriptomic RNA modification and an oligonucleotide portion that is complementary to a portion of the first probe; and
   iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix;
e) sequencing the concatenated amplicons, or a portion thereof, embedded in the polymeric matrix to determine the identity and location of each epitranscriptomically modified RNA of interest in the cell; and
f) administering a treatment for the disease or disorder to the subject if a difference in the profile of epitranscriptomic RNA modifications in the cell relative to one or more non-diseased cells is observed.

In some embodiments, the present disclosure provides methods for treating a disease or disorder in a subject comprising the steps of:
a) contacting a cell (or in multiple cells, e.g., in an intact tissue) taken from the subject with one or more pairs of probes, wherein each pair of probes comprises a first probe (i.e., the “padlock probe”) and a second probe (i.e., the “primer probe”), wherein:
   i) the first probe comprises an oligonucleotide portion that is complementary to the second probe, an oligonucleotide portion that is complementary to an RNA of interest, and an oligonucleotide barcode sequence (e.g., a unique sequence used to identify each RNA of interest, for example, by SEDAL sequencing as discussed herein); and
   ii) the second probe comprises a portion that recognizes an epitranscriptomic RNA modification and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix;
e) sequencing the concatenated amplicons, or a portion thereof, embedded in the polymeric matrix to determine the identity and location of each epitranscriptomically modified RNA of interest in the cell; and
f) administering a treatment for the disease or disorder to the subject if a difference in the profile of epitranscriptomic RNA modifications in the cell relative to one or more non-diseased cells is observed.

In some embodiments, the present disclosure provides methods for treating a disease or disorder in a subject comprising the steps of:
a) contacting a cell taken from a subject with one or more sets of probes, wherein each set of probes comprises a first probe, a second probe, and a third probe wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an RNA-binding protein and an oligonucleotide portion that is complementary to a portion of the first probe; and
   iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix;
e) sequencing the concatenated amplicons, or a portion thereof, embedded in the polymeric matrix to determine the identity and location of each RNA of interest bound by an RNA-binding protein in the cell; and
f) administering a treatment for the disease or disorder to the subject if a difference in the profile of interactions between RNA and RNA-binding proteins in the cell relative to one or more non-diseased cells is observed.

In some embodiments, epitranscriptomic RNA modification in one or more non-diseased cells, or the interactions between RNAs and RNA-binding proteins in one or more non-diseased cells, is profiled simultaneously using the methods disclosed herein as a control experiment. In some embodiments, the epitranscriptomic RNA modification data in one or more non-diseased cells, or the profile of interactions between RNAs and RNA-binding proteins in one or more non-diseased cells, that is compared to the profile of a diseased cell comprises reference data from a time the method was performed on a non-diseased cell previously.

Any suitable treatment for a disease or disorder may be administered to the subject. In some embodiments, the treatment comprises administering a therapeutic agent. In some embodiments, the treatment comprises surgery. In some embodiments, the treatment comprises imaging. In some embodiments, the treatment comprises performing further diagnostic methods. In some embodiments, the treatment comprises radiation therapy. In some embodiments, the therapeutic agent is a small molecule, a protein, a peptide, a nucleic acid, a lipid, or a carbohydrate. In some embodiments, the therapeutic agent is a known drug and/or an FDA-approved drug. In certain embodiments, the protein is an antibody. In certain embodiments, the protein is an antibody variant. In certain embodiments, the protein is a receptor, or a fragment or variant thereof. In certain embodiments, the protein is a cytokine. In certain embodiments, the nucleic acid is an mRNA, an antisense RNA, an miRNA, an siRNA, an RNA aptamer, a double stranded RNA (dsRNA), a short hairpin RNA (shRNA), or an antisense oligonucleotide (ASO).

Treatment of any disease or disorder is contemplated by the methods described herein. In some embodiments, the disease or disorder is a genetic disease, a proliferative disease, an inflammatory disease, an autoimmune disease, a liver disease, a spleen disease, a lung disease, a hematological disease, a neurological disease, a gastrointestinal (GI) tract disease, a genitourinary disease, an infectious disease, a musculoskeletal disease, an endocrine disease, a metabolic disorder, an immune disorder, a central nervous system (CNS) disorder, a neurological disorder, an ophthalmic disease, or a cardiovascular disease.

In some embodiments, the subject is a human. In some embodiments, the sample comprises a biological sample. In some embodiments, the sample comprises a tissue sample. In certain embodiments, the tissue sample is a biopsy (e.g., bone, bone marrow, breast, gastrointestinal tract, lung, liver, pancreas, prostate, brain, nerve, renal, endometrial, cervical, lymph node, muscle, or skin biopsy). In certain embodiments, the biopsy is a tumor biopsy. In certain embodiments, the biopsy is a solid tumor biopsy. In some embodiments, the tissue sample is a brain tissue sample. In certain embodiments, the tissue sample is a central nervous system tissue sample. In certain embodiments, the epitranscriptomic profile of the biological sample informs prognostic decisions that guide therapies including but not limited to, pharmacological interventions for treating various conditions such as diabetes, psychiatric disorders, liver disease, kidney disease, blood disease, endocrine or exocrine disorders, heart disease, cancer therapies such as chemotherapy, targeted therapies, immunotherapy (e.g., checkpoint inhibition, CAR-T, cancer vaccines, etc.), metabolic disorders, or immune and autoimmune disorders.

### Methods of Screening for an Agent Capable of Modulating Epitranscriptomic Modification of One or More RNAs

In another aspect, the present disclosure provides methods for screening for an agent capable of modulating epitranscriptomic modification of one or more RNAs of interest or modulating interactions between RNA-binding proteins and RNAs or one or more RNAs of interest. For example, the methods for profiling epitranscriptomic RNA modifications or interactions between RNA-binding proteins and RNAs described herein may be performed in a cell (or in multiple cells, e.g., in an intact tissue) in the presence of one or more candidate agents. The expression of various epitranscriptomically modified RNAs of interest or RNAs of interest bound by an RNA-binding protein in the cell (e.g., a normal cell, or a diseased cell) can then be compared to the expression of the same RNAs of interest in a cell that was not exposed to the one or more candidate agents. Any difference in the profile of epitranscriptomic RNA modification or the profile of interactions between RNA-binding proteins and RNAs relative to the cell that was not exposed to the candidate agent(s) may indicate that epitranscriptomic modification of the one or more RNAs of interest, or the interactions between the one or more RNAs of interest and one or more RNA-binding proteins, is modulated by the candidate agent(s). In some embodiments, a particular signature (e.g., of epitranscriptomic modification of multiple RNAs of interest, or of the interaction of an RNA-binding protein with multiple RNAs of interest) that is known to be associated with treatment of a disease may be used to identify a candidate agent capable of modulating epitranscriptomic RNA modification in a desired manner. The methods described herein may also be used to identify drugs that have certain side effects, for example, by looking for specific epitranscriptomic RNA modification signatures or signatures of the interactions between an RNA-binding protein and RNAs of interest when one or more cells is treated with a candidate agent or known drug.

In some embodiments, the present disclosure provides methods for screening for an agent capable of modulating epitranscriptomic modification of one or more RNAs comprising the steps of:
a) contacting a cell that is being treated with or has been treated with a candidate agent with one or more sets of probes, wherein each set of probes comprises a first probe, a second probe, and a third probe wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an epitranscriptomic RNA modification and an oligonucleotide portion that is complementary to a portion of the first probe; and
   iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell;

wherein a difference in the profile of epitranscriptomic RNA modifications in the cell in the presence of the candidate agent relative to in the absence of the candidate agent indicates that the candidate agent modulates epitranscriptomic RNA modification.

In some embodiments, the present disclosure provides methods for screening for an agent capable of modulating epitranscriptomic modification of one or more RNAs comprising the steps of:
a) contacting a cell (or in multiple cells, e.g., in an intact tissue) that is being treated with or has been treated with a candidate agent (or combinations of multiple candidate agents and/or known drugs, e.g., as provided in a screening library of compounds) with one or more pairs of probes, wherein each pair of probes comprises a first probe (i.e., the “padlock probe”) and a second probe (i.e., the “primer probe”), wherein:
   i) the first probe comprises an oligonucleotide portion that is complementary to the second probe, an oligonucleotide portion that is complementary to an RNA of interest, and an oligonucleotide barcode sequence (e.g., a unique sequence used to identify each RNA of interest, for example, by SEDAL sequencing as discussed herein); and
   ii) the second probe comprises a portion that recognizes an epitranscriptomic RNA modification and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell;
wherein a difference in the profile of epitranscriptomic RNA modifications in the cell in the presence of the candidate agent relative to in the absence of the candidate agent indicates that the candidate agent modulates epitranscriptomic RNA modification.

In some embodiments, the present disclosure provides methods for screening for an agent capable of modulating interactions between RNA and RNA-binding proteins, the method comprising:
a) contacting a cell taken from a subject with one or more sets of probes, wherein each set of probes comprises a first probe, a second probe, and a third probe wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an RNA-binding protein and an oligonucleotide portion that is complementary to a portion of the first probe; and
   iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each RNA of interest bound by an RNA-binding protein in the cell;
wherein a difference in the profile of interactions between RNA and RNA-binding proteins in the presence of the candidate agent relative to in the absence of the candidate agent indicates that the candidate agent modulates interactions between RNA and RNA-binding proteins.

In some embodiments, the candidate agent is a small molecule, a protein, a peptide, a nucleic acid, a lipid, or a carbohydrate. In some embodiments, the candidate agent comprises a known drug or an FDA-approved drug. In certain embodiments, the protein is an antibody. In certain embodiments, the protein is an antibody fragment or an antibody variant. In certain embodiments, the protein is a receptor. In certain embodiments, the protein is a cytokine. In certain embodiments, the nucleic acid is an mRNA, an antisense RNA, an miRNA, an siRNA, an RNA aptamer, a double stranded RNA (dsRNA), a short hairpin RNA (shRNA), or an antisense oligonucleotide (ASO). In some embodiments, multiple candidate agents are provided as a screening library. Any candidate agent may be screened using the methods described herein. In particular, any candidate agents thought to be capable of modulating epitranscriptomic modification of one or more RNAs of interest or interaction of one or more RNAs of interest with an RNA-binding protein may be screened using the methods described herein. In some embodiments, modulation of epitranscriptomic modification of one or more RNAs of interest and/or interaction of one or more RNAs of interest with an RNA-binding protein by the candidate agent is associated with reducing, relieving, or eliminating the symptoms of a disease or disorder, or preventing the development or progression of the disease or disorder. In some embodiments, the disease or disorder is a genetic disease, a proliferative disease, an inflammatory disease, an autoimmune disease, a liver disease, a spleen disease, a lung disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, a central nervous system (CNS) disorder, or a cardiovascular disease.

### Probes

The present disclosure also provides pairs of probes for use in the methods and systems for profiling epitranscriptomic RNA modifications described herein. In one aspect, the present disclosure provides pairs of probes comprising a first probe (also referred to herein as the “padlock” probe) and a second probe (also referred to herein as the “primer” probe), wherein:
i) the first probe comprises an oligonucleotide portion that is complementary to the second probe, an oligonucleotide portion that is complementary to an RNA of interest, and an oligonucleotide barcode sequence (e.g., a unique sequence used to identify each RNA of interest, for example, by SEDAL sequencing as discussed herein); and
ii) the second probe comprises a portion that recognizes an epitranscriptomic RNA modification and an oligonucleotide portion that is complementary to a portion of the first probe.

All of the probes described herein may optionally have spacers or linkers of various nucleotide lengths in between each of the recited components, or the components of the oligonucleotide probes may be joined directly to one another (i.e., by a phosphodiester bond). All of the probes described herein may comprise standard nucleotides, or some of the standard nucleotides may be substituted for any modified nucleotides known in the art.

The second probe of the pair of probes (also referred to herein as the “primer probe”) comprises a portion that recognizes an epitranscriptomic RNA modification (i.e., a post-transcriptional modification present on a particular RNA of interest). In some embodiments, the portion of the second probe that binds the epitranscriptomic RNA modification comprises a peptide. In some embodiments, the portion of the second probe that binds the epitranscriptomic RNA modification comprises an aptamer. In some embodiments, the portion of the second probe that binds the epitranscriptomic RNA modification comprises a small molecule. In certain embodiments, the second probe binds to the epitranscriptomic modification through a mechanism comprising a biotin-streptavidin interaction. The portion of the probe that recognizes an epitranscriptomic RNA modification may be a protein (e.g., an antibody or antibody variant, or any protein that is otherwise capable of binding to a specific epitranscriptomic modification). In some embodiments, the protein is PAPG. In certain embodiments, PAPG recognizes and binds an antibody that recognizes an epitranscriptomic RNA modification. In some embodiments, the portion of the second probe that recognizes the epitranscriptomic RNA modification comprises an agent that binds an antibody, or an antibody variant. For example, the second probe may comprise a secondary antibody, and a primary antibody may be used to bind to the epitranscriptomic RNA modification. The secondary antibody on the second probe then recognizes the primary antibody bound to the epitranscriptomic RNA modification. See, for example, FIG. 1. In certain embodiments, the portion of the second probe that recognizes the epitranscriptomic RNA modification comprises an antibody (e.g., a secondary antibody), or an antibody variant. When the portion of the second probe that recognizes the epitranscriptomic RNA modification is a secondary antibody, the secondary antibody may bind a primary antibody that recognizes the epitranscriptomic RNA modification. In some embodiments, the primary antibody is an anti-m⁶A antibody, an anti-m¹A antibody, an anti-pseudouridine antibody, an anti-m⁶Am antibody, an anti-m⁷G antibody, an anti-ac⁴C antibody, an anti-Nm antibody, or an anti-m⁵C antibody. Other epitranscriptomic modifications include those described in Kumar, S. et al., Frontiers in Cell and Developmental Biology. 9 (2021); and Harcourt, E. M. et al., Nature. 541, 339-346 (2017), each of which is incorporated herein by reference. In place of an antibody, the present disclosure also contemplates the use of any agent capable of binding an epitranscriptomic RNA modification on the probes described herein. In some embodiments, the portion of the second probe that binds the epitranscriptomic RNA modification comprises a protein. In certain embodiments, the protein is an m⁶A-specific YTH domain protein, or a portion thereof. In some embodiments, the portion of the second probe that binds the epitranscriptomic RNA modification comprises an antibody, or an antibody variant.

In addition to the portion that recognizes the epitranscriptomic RNA modification, the second probe also comprises a portion that is complementary to a portion of the first probe. In the pairs of probes provided herein, the portions of the first and second probes that are complementary to one another may be the same on each set of probes. In some embodiments, the portions of the first and second probes that are complementary to one another are unique on each pair of probes. In some embodiments, the portion of the second probe that is complementary to a portion of the first probe is about 3-20, about 4-19, about 5-18, about 6-17, about 7-16, about 8-15, about 9-14, about 10-13, or about 11-12 nucleotides in length. In some embodiments, the portion of the second probe that is complementary to a portion of the first probe is about 3, about 4, about 5, about 6, about 7, about 8, about 9, about 10, about 11, about 12, about 13, about 14, about 15, about 16, about 17, about 18, about 19, or about 20 nucleotides in length.

In some embodiments, each portion of the second probe is connected by an optional linker. In certain embodiments, the optional linker is 1, 2, 3, 4, 5, 6, 7, 8, 9, or 10 nucleotides long. In some embodiments, the linker is a non-nucleotide linker (e.g., amino acids, chemical linkers, polymers, etc.). In some embodiments, the second probe of the pair of probes comprises the structure:
5′-[portion recognizing epitranscriptomic RNA modification]-[portion complementary to first probe]-3′,
   wherein]-[comprises an optional linker (e.g., a nucleotide linker). In some embodiments,]-[represents a direct linkage between two portions of the second probe (i.e., a phosphodiester bond).

The first probe of the pair of probes provided herein (also referred to herein as the “padlock” probe) includes an oligonucleotide portion that is complementary to the second probe. In some embodiments, the portion of the first probe that is complementary to a portion of the second probe is about 3-20, about 4-19, about 5-18, about 6-17, about 7-16, about 8-15, about 9-14, about 10-13, or about 11-12 nucleotides in length. In some embodiments, the portion of the first probe that is complementary to a portion of the second probe is about 3, about 4, about 5, about 6, about 7, about 8, about 9, about 10, about 11, about 12, about 13, about 14, about 15, about 16, about 17, about 18, about 19, or about 20 nucleotides in length. In certain embodiments, the oligonucleotide portion of the first probe that is complementary to the second probe is split between the 5′ end and the 3′ end of the first probe.

The first probe of the pair of probes provided herein also comprises an oligonucleotide portion that is complementary to an RNA of interest. In some embodiments, the oligonucleotide portion of the first probe that is complementary to the RNA of interest is about 10-30, about 11-29, about 12-28, about 13-27, about 14-26, about 15-25, about 16-24, about 17-23, about 18-22, or about 19-21 nucleotides in length. In some embodiments, the oligonucleotide portion of the first probe that is complementary to the RNA of interest is about 10, about 11, about 12, about 13, about 14, about 15, about 16, about 17, about 18, about 19, about 20, about 21, about 22, about 23, about 24, about 25, about 26, about 27, about 28, about 29, about 30, or more than 30 nucleotides in length.

The first probe of the pair of probes disclosed herein also comprises an oligonucleotide barcode sequence made up of a specific sequence of nucleotides. In some embodiments, the oligonucleotide barcode sequence of the first probe is about 1-10, about 2-9, about 3-8, about 4-7, or about 5-6 nucleotides in length. In some embodiments, the oligonucleotide barcode sequence of the first probe is about 1, about 2, about 3, about 4, about 5, about 6, about 7, about 8, about 9, or about 10 nucleotides in length. The barcodes of the oligonucleotide probes described herein may comprise gene-specific sequences used to identify RNAs of interest (i.e., RNAs that have been modified with at least one epitranscriptomic modification).

The arrangement of the portions of the first oligonucleotide probe in any order is contemplated by the present disclosure. In some embodiments, each portion of the first probe is connected by an optional linker. In some embodiments, the optional linker is a nucleotide linker. In certain embodiments, the optional linker is 1, 2, 3, 4, 5, 6, 7, 8, 9, or 10 nucleotides long. In some embodiments, the optional linkers comprise other types of linkages besides nucleotides (e.g., chemical linkers, peptide linkers, etc.). In some embodiments, the first probe comprises the structure:
5′-[portion complementary to second probe]-[portion complementary to RNA of interest]-[barcode sequence]-[portion complementary to second probe]-3′;
5′-[portion complementary to second probe]-[barcode sequence]-[portion complementary to RNA of interest]-[portion complementary to second probe]-3′;
5′-[portion complementary to second probe]-[portion complementary to RNA of interest]-[barcode sequence]-3′;
5′-[portion complementary to second probe]-[barcode sequence]-[portion complementary to RNA of interest]-3′;
5′-[portion complementary to RNA of interest]-[barcode sequence]-[portion complementary to second probe]-3′; or
5′-[barcode sequence]-[portion complementary to RNA of interest]-[portion complementary to second probe]-3′,
   wherein ]-[comprises an optional nucleotide linker. In some embodiments,]-[represents a direct linkage between two portions of the first probe (i.e., a phosphodiester bond). In some embodiments, any of the oligonucleotide portions of the first probe and/or the second probe comprise DNA.

The present disclosure also provides sets of probes for use in the methods and systems for profiling epitranscriptomic RNA modifications described herein. In one aspect, the present disclosure provides pairs of probes comprising a first probe (also referred to herein as the “padlock” probe) and a second probe (also referred to herein as the “splint” probe), and a third probe (also referred to herein as the “primer” probe), wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an epitranscriptomic RNA modification and an oligonucleotide portion that is complementary to a portion of the first probe; and
iii) the third probe comprises an oligonucleotide portion that is complementary to the RNA of interest and an oligonucleotide portion that is complementary to a portion of the first probe.

All of the probes in the sets of probes described herein may optionally have spacers or linkers of various nucleotide lengths in between each of the recited components, or the components of the oligonucleotide probes may be joined directly to one another (i.e., by a phosphodiester bond). All of the probes described herein may comprise standard nucleotides, or some of the standard nucleotides may be substituted for any modified nucleotides known in the art.

The second probe of the set of probes (also referred to herein as the “splint probe”) comprises a portion that recognizes an epitranscriptomic RNA modification (i.e., a post-transcriptional modification present on a particular RNA of interest). In some embodiments, the portion of the second probe that binds the epitranscriptomic RNA modification comprises a peptide. In some embodiments, the portion of the second probe that binds the epitranscriptomic RNA modification comprises an aptamer. In some embodiments, the portion of the second probe that binds the epitranscriptomic RNA modification comprises a small molecule. In certain embodiments, the second probe binds to the epitranscriptomic modification through a mechanism comprising a biotin-streptavidin interaction. The portion of the probe that recognizes an epitranscriptomic RNA modification may be a protein (e.g., an antibody or antibody variant, or any protein that is otherwise capable of binding to a specific epitranscriptomic modification). In certain embodiments, the protein is PAPG. In some embodiments, the portion of the second probe that recognizes the epitranscriptomic RNA modification comprises an agent that binds an antibody, or an antibody variant. For example, the second probe may comprise a secondary antibody, and a primary antibody may be used to bind to the epitranscriptomic RNA modification. The secondary antibody on the second probe then recognizes the primary antibody bound to the epitranscriptomic RNA modification. See, for example, FIG. 1. In another example, the second probe may comprise the protein PAPG, which may bind an antibody that binds to the epitranscriptomic RNA modification. In some embodiments, PAPG recognizes and binds to an antibody, and the antibody recognizes and binds to the epitranscriptomic RNA modification. In certain embodiments, the portion of the second probe that recognizes the epitranscriptomic RNA modification comprises an antibody (e.g., a secondary antibody), or an antibody variant. In some embodiments, the secondary antibody recognizes and binds to a primary antibody that recognizes and binds to the epitranscriptomic RNA. The cell may be contacted with the primary antibody before or after being contacted with the one or more pairs of probes. In certain embodiments, the cell is contacted with the primary antibody prior to being contacted with the one or more pairs of probes. In some embodiments, the primary antibody is an anti-m⁶A antibody, an anti-m¹A antibody, an anti-pseudouridine antibody, an anti-m⁶Am antibody, an anti-m⁷G antibody, an anti-ac⁴C antibody, an anti-Nm antibody, or an anti-m⁵C antibody. In place of a primary antibody, the present disclosure also contemplates the use of any agent capable of binding an epitranscriptomic RNA modification directly on the probes described herein. In some embodiments, the portion of the second probe that binds the epitranscriptomic RNA modification directly comprises a protein. In certain embodiments, the protein is an m⁶A-specific YTH domain protein. In some embodiments, the portion of the second probe that binds the epitranscriptomic RNA modification directly comprises an antibody, or an antibody variant.

In some embodiments, the second probe of the set of probes further comprises a polymerization blocker. The polymerization blocker can be any moiety capable of preventing the use of the second oligonucleotide probe as a primer in the rolling circle amplification of step (c) of the methods described herein. In some embodiments, the polymerization blocker is at the 3′ end of the second oligonucleotide probe. The polymerization blocker can be, for example, any chemical moiety that prevents a polymerase from using the second oligonucleotide probe as a primer for polymerization. In some embodiments, the polymerization blocker is a nucleic acid residue comprising a blocked 3′ hydroxyl group (e.g., comprising an oxygen protecting group on the 3′ hydroxyl group). In some embodiments, the polymerization blocker comprises a hydrogen in place of the 3′ hydroxyl group. In some embodiments, the polymerization blocker comprises any chemical moiety in place of the 3′ hydroxyl group that prevents an additional nucleotide from being added. In some embodiments, the polymerization blocker comprises an inverted nucleic acid residue. In some embodiments, the polymerization blocker is an inverted adenosine, thymine, cytosine, guanosine, or uridine residue. In certain embodiments, the polymerization blocker is an inverted thymine residue.

In addition to the portion that recognizes the epitranscriptomic RNA modification, the second probe also comprises a portion that is complementary to a portion of the first probe. In the sets of probes provided herein, the portions of the first and second probes that are complementary to one another may be the same on each set of probes. In some embodiments, the portions of the first and second probes that are complementary to one another are unique on each of the first and second probes. In some embodiments, the portion of the second probe that is complementary to a portion of the first probe is about 3-20, about 4-19, about 5-18, about 6-17, about 7-16, about 8-15, about 9-14, about 10-13, or about 11-12 nucleotides in length. In some embodiments, the portion of the second probe that is complementary to a portion of the first probe is about 3, about 4, about 5, about 6, about 7, about 8, about 9, about 10, about 11, about 12, about 13, about 14, about 15, about 16, about 17, about 18, about 19, or about 20 nucleotides in length.

In some embodiments, each portion of the second probe is connected by an optional linker. In some embodiments, the optional linker is a nucleotide linker. In certain embodiments, the optional linker is 1, 2, 3, 4, 5, 6, 7, 8, 9, or 10 nucleotides long. In some embodiments, the second probe of the sets of probes described herein comprises the structure:
5′-[portion recognizing epitranscriptomic RNA modification]-[portion complementary to first probe]-3′,
   wherein]-[comprises an optional linker (e.g., a nucleotide linker). In some embodiments,]-[represents a direct linkage between two portions of the second probe (i.e., a phosphodiester bond).

The first probe of the sets of probes described herein (also referred to herein as the “padlock” probe) includes an oligonucleotide portion that is complementary to the second probe. In some embodiments, the portion of the first probe that is complementary to a portion of the second probe is about 4-20, about 5-19, about 6-18, about 7-17, about 8-16, about 9-15, about 10-14, or about 11-13 nucleotides in length. In some embodiments, the portion of the first probe that is complementary to a portion of the second probe is about 4, about 5, about 6, about 7, about 8, about 9, about 10, about 11, about 12, about 13, about 14, about 15, about 16, about 17, about 18, about 19, or about 20 nucleotides in length. In certain embodiments, the oligonucleotide portion of the first probe that is complementary to the second probe is split between the 5′ end and the 3′ end of the first probe.

The first probe of the sets of probes disclosed herein also comprises an oligonucleotide portion that is complementary to an RNA of interest. In some embodiments, the oligonucleotide portion of the first probe that is complementary to the RNA of interest is about 10-30, about 11-29, about 12-28, about 13-27, about 14-26, about 15-25, about 16-24, about 17-23, about 18-22, or about 19-21 nucleotides in length. In some embodiments, the oligonucleotide portion of the first probe that is complementary to the RNA of interest is about 10, about 11, about 12, about 13, about 14, about 15, about 16, about 17, about 18, about 19, about 20, about 21, about 22, about 23, about 24, about 25, about 26, about 27, about 28, about 29, about 30, or more than 30 nucleotides in length.

The first probe of the sets of probes disclosed herein also comprises an oligonucleotide barcode sequence made up of a specific sequence of nucleotides. In some embodiments, the oligonucleotide barcode sequence of the first probe is about 1-10, about 2-9, about 3-8, about 4-7, or about 5-6 nucleotides in length. In some embodiments, the oligonucleotide barcode sequence of the first probe is about 1, about 2, about 3, about 4, about 5, about 6, about 7, about 8, about 9, or about 10 nucleotides in length. The barcodes of the oligonucleotide probes described herein may comprise gene-specific sequences used to identify RNAs of interest (i.e., RNAs that have been modified with at least one particular epitranscriptomic modification).

The arrangement of the portions of the first oligonucleotide probe in any order is contemplated by the present disclosure. In some embodiments, a portion of the first probe is connected by an optional linker to another portion. In certain embodiments, the optional linker is 1, 2, 3, 4, 5, 6, 7, 8, 9, or 10 nucleotides long. In some embodiments, the first probe comprises the structure:
5′-[portion complementary to second probe]-[oligonucleotide barcode sequence]-[portion complementary to RNA of interest]-[portion complementary to third probe]-[portion complementary to second probe]-3′,
   wherein]-[comprises an optional nucleotide linker. In some embodiments,]-[represents a direct linkage between two portions of the first probe (i.e., a phosphodiester bond). In some embodiments, any of the oligonucleotide portions of the probes that make up the sets of probes provided herein comprise DNA.

The third probe of the sets of probes provided herein (also referred to as the “primer probe”) comprises a portion complementary to an RNA of interest and a portion complementary to the first probe of the set of probes. In some embodiments, the portion of the third probe that is complementary to the RNA of interest is 10-30, 11-29, 12-28, 13-27, 14-26, 15-25, 16-24, 17-23, 18-22, or 19-21 nucleotides in length. In some embodiments, the portion of the third probe that is complementary to the RNA of interest is 10, 11, 12, 13, 14, 15, 16, 17, 18, 19, 20, 21, 22, 23, 24, 25, 26, 27, 28, 29, or 30 nucleotides in length. In some embodiments, the portion of the third probe that is complementary to a portion of the first probe is 5-15, 6-14, 7-13, 8-12, or 9-11 nucleotides in length. In some embodiments, the portion of the third probe that is complementary to a portion of the first probe is 5, 6, 7, 8, 9, 10, 11, 12, 13, 14, or 15 nucleotides in length.

In certain embodiments, the third probe of the set of probes comprises the structure:

5′-[portion complementary to RNA of interest]-[portion complementary to first probe]-3′, wherein ]-[comprises an optional nucleotide linker. In some embodiments,]-[represents a direct linkage between two portions of the first probe (i.e., a phosphodiester bond).

The present disclosure also provides sets of probes for use in the methods and systems for profiling interactions between RNA-binding proteins and RNAs described herein. In one aspect, the present disclosure provides pairs of probes comprising a first probe (also referred to herein as the “padlock” probe) and a second probe (also referred to herein as the “splint” probe), and a third probe (also referred to herein as the “primer” probe), wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an RNA-binding protein and an oligonucleotide portion that is complementary to a portion of the first probe; and
iii) the third probe comprises an oligonucleotide portion that is complementary to the RNA of interest and an oligonucleotide portion that is complementary to a portion of the first probe.

The second probe of the set of probes for profiling interactions between RNA-binding proteins and RNAs (also referred to herein as the “splint probe”) comprises a portion that recognizes an RNA-binding protein (e.g., an enzyme that introduces an epitranscriptomic modification onto an RNA). In some embodiments, the portion of the second probe that binds the RNA-binding protein comprises a peptide. In some embodiments, the portion of the second probe that binds the RNA-binding protein comprises an aptamer. In some embodiments, the portion of the second probe that binds the RNA-binding protein comprises a small molecule. In certain embodiments, the second probe binds to the RNA-binding protein through a mechanism comprising a biotin-streptavidin interaction. The portion of the probe that recognizes an RNA-binding protein may be a protein (e.g., an antibody or antibody variant, or any protein that is otherwise capable of binding to a specific RNA-binding protein). In some embodiments, the portion of the second probe that recognizes the RNA-binding protein comprises an agent that binds an antibody, or an antibody variant. For example, the second probe may comprise a secondary antibody, and a primary antibody may be used to bind to the RNA-binding protein. The secondary antibody on the second probe then recognizes the primary antibody bound to the RNA-binding protein. See, for example, FIGS. 13A and 13C. In certain embodiments, the portion of the second probe that recognizes the RNA-binding protein comprises an antibody (e.g., a secondary antibody), or an antibody variant. In some embodiments, the secondary antibody recognizes and binds to a primary antibody that recognizes the RNA-binding protein. The cell may be contacted with the primary antibody before or after being contacted with the one or more pairs of probes. In certain embodiments, the cell is contacted with the primary antibody prior to being contacted with the one or more pairs of probes. In place of a primary antibody, the present disclosure also contemplates the use of any agent capable of recognizing and directly binding an RNA-binding protein on the probes described herein. In some embodiments, the portion of the second probe that binds the RNA-binding protein directly comprises a protein. In some embodiments, the portion of the second probe that binds the RNA-binding protein directly comprises an antibody, or an antibody variant.

In some embodiments, the second probe of the set of probes further comprises a polymerization blocker. The polymerization blocker can be any moiety capable of preventing the use of the second oligonucleotide probe as a primer in the rolling circle amplification of step (c) of the methods described herein. In some embodiments, the polymerization blocker is at the 3′ end of the second oligonucleotide probe. The polymerization blocker can be, for example, any chemical moiety that prevents a polymerase from using the second oligonucleotide probe as a primer for polymerization. In some embodiments, the polymerization blocker is a nucleic acid residue comprising a blocked 3′ hydroxyl group (e.g., comprising an oxygen protecting group on the 3′ hydroxyl group). In some embodiments, the polymerization blocker comprises a hydrogen in place of the 3′ hydroxyl group. In some embodiments, the polymerization blocker comprises any chemical moiety in place of the 3′ hydroxyl group that prevents an additional nucleotide from being added. In some embodiments, the polymerization blocker comprises an inverted nucleic acid residue. In some embodiments, the polymerization blocker is an inverted adenosine, thymine, cytosine, guanosine, or uridine residue. In certain embodiments, the polymerization blocker is an inverted thymine residue.

In addition to the portion that recognizes the RNA-binding protein, the second probe also comprises a portion that is complementary to a portion of the first probe. In the sets of probes provided herein, the portions of the first and second probes that are complementary to one another may be the same on each set of probes. In some embodiments, the portions of the first and second probes that are complementary to one another are unique on each of the first and second probes. In some embodiments, the portion of the second probe that is complementary to a portion of the first probe is about 3-20, about 4-19, about 5-18, about 6-17, about 7-16, about 8-15, about 9-14, about 10-13, or about 11-12 nucleotides in length. In some embodiments, the portion of the second probe that is complementary to a portion of the first probe is about 3, about 4, about 5, about 6, about 7, about 8, about 9, about 10, about 11, about 12, about 13, about 14, about 15, about 16, about 17, about 18, about 19, or about 20 nucleotides in length.

In some embodiments, each portion of the second probe is connected by an optional linker. In some embodiments, the optional linker is a nucleotide linker. In certain embodiments, the optional linker is 1, 2, 3, 4, 5, 6, 7, 8, 9, or 10 nucleotides long. In some embodiments, the second probe of the sets of probes comprises the structure:
5′-[portion recognizing RNA-binding protein]-[portion complementary to first probe]-3′,
   wherein]-[comprises an optional linker (e.g., a nucleotide linker). In some embodiments,]-[represents a direct linkage between two portions of the second probe (i.e., a phosphodiester bond).

The first probe of the sets of probes used in the methods for profiling interactions between RNA-binding proteins and RNAs described herein (also referred to herein as the “padlock” probe) includes an oligonucleotide portion that is complementary to the second probe. In some embodiments, the portion of the first probe that is complementary to a portion of the second probe is about 4-20, about 5-19, about 6-18, about 7-17, about 8-16, about 9-15, about 10-14, or about 11-13 nucleotides in length. In some embodiments, the portion of the first probe that is complementary to a portion of the second probe is about 4, about 5, about 6, about 7, about 8, about 9, about 10, about 11, about 12, about 13, about 14, about 15, about 16, about 17, about 18, about 19, or about 20 nucleotides in length. In certain embodiments, the oligonucleotide portion of the first probe that is complementary to the second probe is split between the 5′ end and the 3′ end of the first probe.

The first probe of the sets of probes used in the methods for profiling interactions between RNA-binding proteins and RNAs disclosed herein also comprises an oligonucleotide portion that is complementary to an RNA of interest. In some embodiments, the oligonucleotide portion of the first probe that is complementary to the RNA of interest is about 10-30, about 11-29, about 12-28, about 13-27, about 14-26, about 15-25, about 16-24, about 17-23, about 18-22, or about 19-21 nucleotides in length. In some embodiments, the oligonucleotide portion of the first probe that is complementary to the RNA of interest is about 10, about 11, about 12, about 13, about 14, about 15, about 16, about 17, about 18, about 19, about 20, about 21, about 22, about 23, about 24, about 25, about 26, about 27, about 28, about 29, about 30, or more than 30 nucleotides in length.

The first probe of the sets of probes disclosed herein also comprises an oligonucleotide barcode sequence made up of a specific sequence of nucleotides. In some embodiments, the oligonucleotide barcode sequence of the first probe is about 1-10, about 2-9, about 3-8, about 4-7, or about 5-6 nucleotides in length. In some embodiments, the oligonucleotide barcode sequence of the first probe is about 1, about 2, about 3, about 4, about 5, about 6, about 7, about 8, about 9, or about 10 nucleotides in length. The barcodes of the oligonucleotide probes described herein may comprise gene-specific sequences used to identify RNAs of interest (i.e., RNAs that are bound by at least one RNA-binding protein).

The arrangement of the portions of the first oligonucleotide probe in any order is contemplated by the present disclosure. In some embodiments, a portion of the first probe is connected by an optional linker to another portion. In certain embodiments, the optional linker is 1, 2, 3, 4, 5, 6, 7, 8, 9, or 10 nucleotides long. In some embodiments, the first probe comprises the structure:
5′-[portion complementary to second probe]-[oligonucleotide barcode sequence]-[portion complementary to RNA of interest]-[portion complementary to third probe]-[portion complementary to second probe]-3′,
   wherein]-[comprises an optional nucleotide linker. In some embodiments,]-[represents a direct linkage between two portions of the first probe (i.e., a phosphodiester bond). In some embodiments, any of the oligonucleotide portions of the probes that make up the sets of probes provided herein comprise DNA.

The third probe of the sets of probes used in the methods for profiling interactions between RNA-binding proteins and RNAs provided herein (also referred to as the “primer probe”) comprises a portion complementary to an RNA of interest and a portion complementary to the first probe of the set of probes. In some embodiments, the portion of the third probe that is complementary to the RNA of interest is 10-30, 11-29, 12-28, 13-27, 14-26, 15-25, 16-24, 17-23, 18-22, or 19-21 nucleotides in length. In some embodiments, the portion of the third probe that is complementary to the RNA of interest is 10, 11, 12, 13, 14, 15, 16, 17, 18, 19, 20, 21, 22, 23, 24, 25, 26, 27, 28, 29, or 30 nucleotides in length. In some embodiments, the portion of the third probe that is complementary to a portion of the first probe is 5-15, 6-14, 7-13, 8-12, or 9-11 nucleotides in length. In some embodiments, the portion of the third probe that is complementary to a portion of the first probe is 5, 6, 7, 8, 9, 10, 11, 12, 13, 14, or 15 nucleotides in length.

In certain embodiments, the third probe of the set of probes comprises the structure:
5′-[portion complementary to RNA of interest]-[portion complementary to first probe]-3′, wherein]-[comprises an optional nucleotide linker. In some embodiments,]-[represents a direct linkage between two portions of the first probe (i.e., a phosphodiester bond).

All of the probes described herein may optionally have spacers or linkers of various nucleotide lengths in between each of the recited components, or the components of the oligonucleotide probes may be joined directly to one another (i.e., by a phosphodiester bond). All of the probes described herein may comprise standard nucleotides, or some of the standard nucleotides may be substituted for any modified nucleotides known in the art.

In some embodiments, the present disclosure provides a plurality of probes comprising multiple pairs of probes or sets of probes as described herein. In certain embodiments, each pair of probes or set of probes in the plurality of probes comprises oligonucleotide portions that are complementary to a different RNA of interest. In some embodiments, the plurality of probes comprises more than 1, more than 2, more than 3, more than 4, more than 5, more than 10, more than 20, more than 30, more than 40, more than 50, more than 100, more than 200, more than 500, more than 1000, more than 2000, or more than 3000 pairs of probes or sets of probes.

In some aspects, the present disclosure provides libraries comprising multiple sets of any of the probes described herein. In some aspects, the present disclosure provides libraries comprising multiple pairs of any of the probes provided herein. In some aspects, the present disclosure provides libraries comprising multiple first probes from the sets or pairs of probes provided herein, multiple second probes from the sets or pairs of probes provided herein, or multiple third probes from the sets of probes provided herein.

### Kits

Also provided by the disclosure are kits. In one aspect, the kits provided may comprise one or more of the probes as described herein. In some embodiments, the kits comprise any of the pairs of probes or sets of probes described herein, or multiple pairs of probes or sets of probes. In certain embodiments, the kits comprise more than 1, more than 2, more than 3, more than 4, more than 5, more than 10, more than 20, more than 30, more than 40, more than 50, more than 100, more than 200, more than 500, more than 1000, more than 2000, or more than 3000 pairs of probes or sets of probes. In some embodiments, the kits may further comprise a container (e.g., a vial, ampule, bottle, and/or dispenser package, or other suitable container). The kits may also comprise cells for performing control experiments. In some embodiments, the kits may further comprise other reagents for performing the methods disclosed herein (e.g., enzymes such as a ligase or a polymerase, amine-modified nucleotides as described herein, primary antibodies, secondary antibodies, buffers, and/or reagents and monomers for making a polymeric matrix (e.g., a polyacrylamide matrix)). In some embodiments, the kits are useful for profiling epitranscriptomic RNA modifications in a cell. In some embodiments, the kits are additionally useful for profiling unmodified RNAs alongside epitranscriptomically-modified RNAs (i.e., the kits also include pairs of probes for profiling unmodified RNAs of interest). In some embodiments, the kits are useful for profiling interactions between RNA-binding proteins and RNAs in a cell. In some embodiments, the kits are useful for diagnosing a disease in a subject. In some embodiments, the kits are useful for screening for an agent capable of modulating epitranscriptomic modification of one or more RNAs. In some embodiments, the kits are useful for diagnosing a disease or disorder in a subject. In some embodiments, the kits are useful for treating a disease or disorder in a subject. In certain embodiments, a kit described herein further includes instructions for using the kit.

### Systems

In one aspect, the present disclosure provides systems for profiling epitranscriptomic RNA modification in a cell. In some embodiments, such a system comprises:
a) a cell (or in multiple cells, e.g., in an intact tissue);
b) one or more pairs of probes comprising a first probe (i.e., the “padlock probe”) and a second probe (i.e., the “primer probe”), wherein:
   i) the first probe comprises an oligonucleotide portion that is complementary to the second probe, an oligonucleotide portion that is complementary to an RNA of interest, and an oligonucleotide barcode sequence (e.g., a unique sequence used to identify each RNA of interest, for example, by SEDAL sequencing as discussed herein); and
   ii) the second probe comprises a portion that recognizes an epitranscriptomic RNA modification and an oligonucleotide portion that is complementary to a portion of the first probe;
c) a microscope; and
d) a computer.

In some embodiments, the present disclosure provides systems comprising:
a) a cell;
b) one or more sets of probes comprising a first probe, a second probe, and a third probe, wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an epitranscriptomic RNA modification and an oligonucleotide portion that is complementary to a portion of the first probe; and
   iii) the third probe comprises an oligonucleotide portion that is complementary to the RNA of interest and an oligonucleotide portion that is complementary to a portion of the first probe;
c) a microscope; and
d) a computer.

In some embodiments, the present disclosure provides systems comprising:
a) a cell;
b) one or more sets of probes comprising a first probe, a second probe, and a third probe, wherein:
   i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
   ii) the second probe comprises a portion that recognizes an RNA-binding protein and an oligonucleotide portion that is complementary to a portion of the first probe; and
   iii) the third probe comprises an oligonucleotide portion that is complementary to the RNA of interest and an oligonucleotide portion that is complementary to a portion of the first probe;
c) a microscope; and
d) a computer.

Any of the probes (i.e., the pairs of probes) described herein may be used in the systems contemplated by the present disclosure. In some embodiments, the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), or 5-methylcytosine (m⁵C). In certain embodiments, the epitranscriptomic RNA modification is N⁶-methyladenosine (m⁶A).

In some embodiments, a system further comprises software running on the computer. In some embodiments, the systems may further comprise other reagents (e.g., enzymes such as a ligase or a polymerase, amine-modified nucleotides as described herein, primary antibodies, secondary antibodies, buffers, and/or reagents and monomers for making a polymeric matrix (e.g., a polyacrylamide matrix)).

### EXAMPLES

### Example 1: Single-Cell Profiling of the Epitranscriptome In Situ

A spectrum of single-cell multi-omics technologies have been developed, covering a wide range of discrete information from chromosome architecture to cell-surface protein display. The methodology for single-cell profiling of epitranscriptomic RNA modifications described herein is unique in the field of single-cell multi-omics technologies due to its remarkable versatility and ability to be adapted for different uses. Various chemical and biotechnological tools, including RNA-hydrogel crosslinking, metabolic labeling, DNA barcoding, and in situ sequencing are combined in the methods described herein to detect epitranscriptomic RNA modifications in a high-throughput manner. The methods described herein are useful for understanding RNA modifications and advancing understanding of human diseases associated with epitranscriptomic modifications.

Chemical modification of the transcriptome plays a crucial role in the regulation of RNA activities, processing (e.g., of pre-mRNA), stability, export, and translation. These chemical modifications of cellular RNA are diverse and ubiquitous, providing an additional layer of complexity in regulating gene expression. Epitranscriptomics has been explored in second-generation sequencing, but to advance understanding of RNA modification, there is need for a strategy to study spatial arrangements of modified RNAs in the native cellular environment, as well as in a multiplexed and high-throughput way. The methods described herein address these two issues through the use of probes conjugating an RNA modification-binding moiety (e.g., an RNA-modification binding antibody) with an oligonucleotide primer. The identity of RNAs with specific epitranscriptomic modifications can thus be sequentially decoded and identified using oligonucleotide barcode sequences present on the probes (FIG. 1).

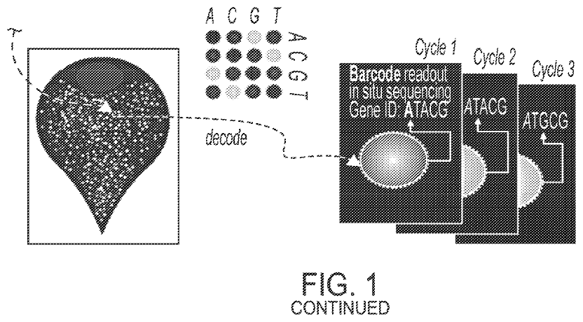

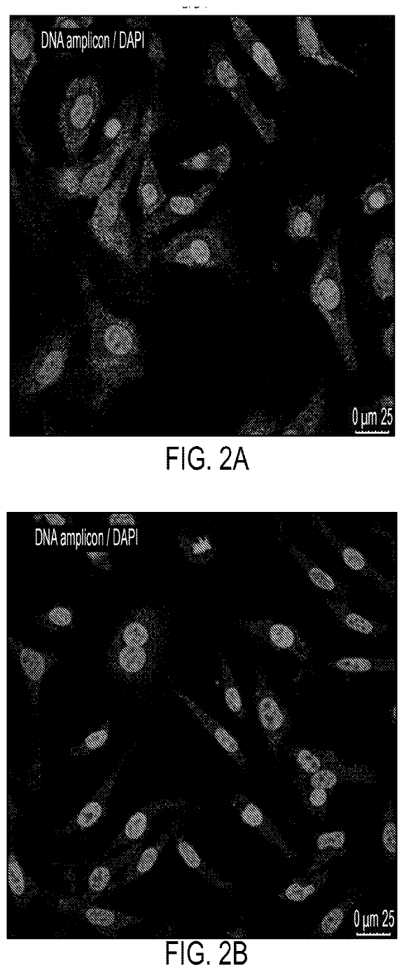

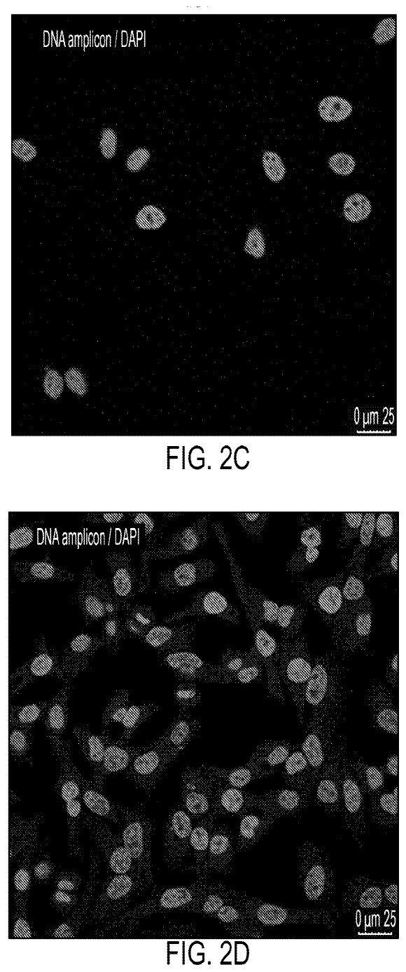

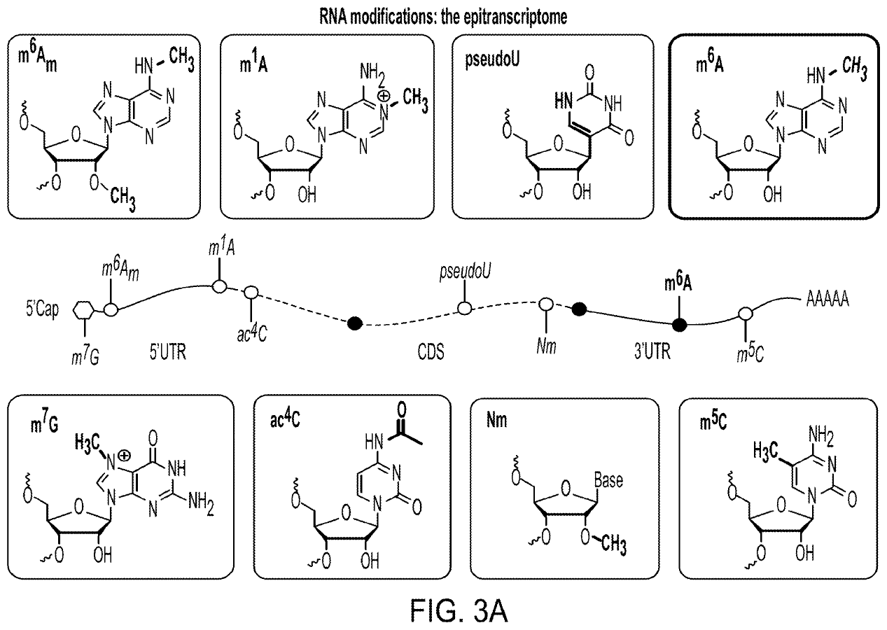

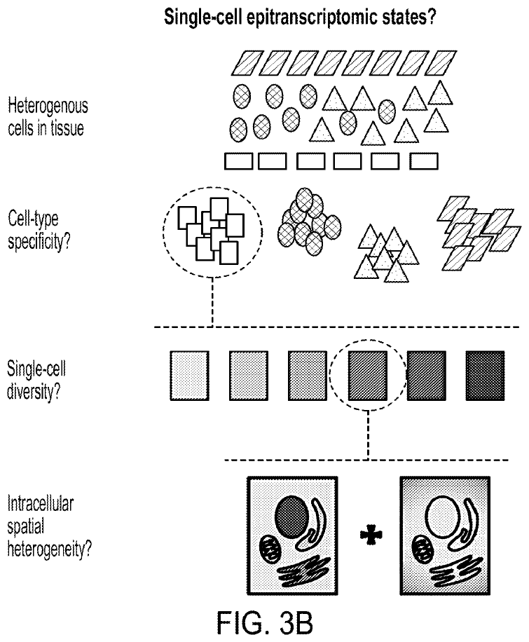

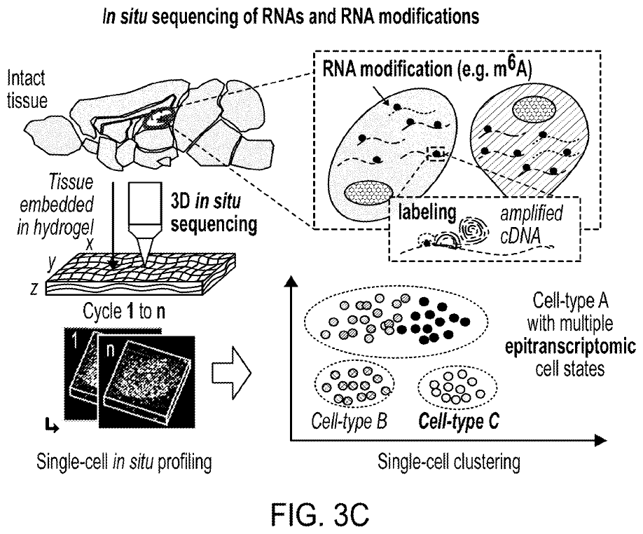

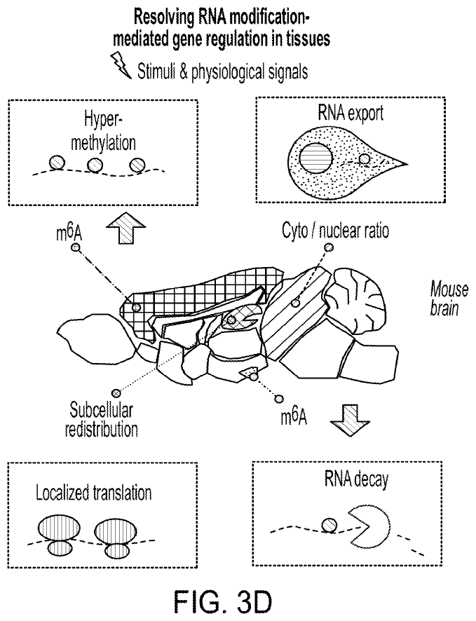

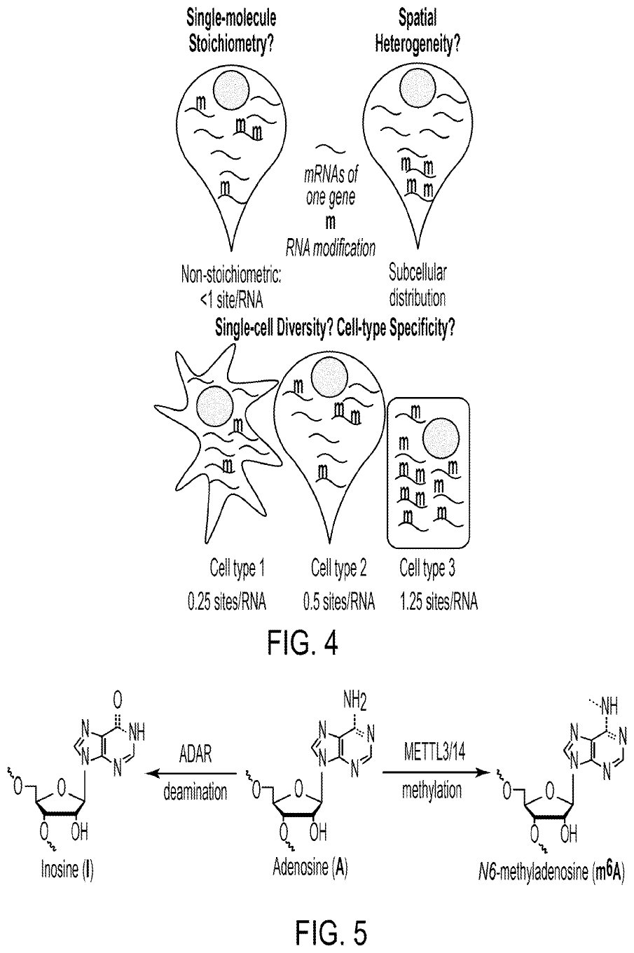

The utility of this method is demonstrated through detection of one of the most abundant and prevalent RNA modifications, N⁶-methyladenosine (m⁶A), a crucial epitranscriptomic marker in mRNA processing, translation, and degradation, m⁶A-modified beta-actin transcripts were detected in cells (FIGS. 2A-2D). Beta-actin is a common and abundant mRNA in cells modified by m⁶A. Raw images show that the signals corresponding to beta-actin mRNAs can be seen (FIG. 2A) in comparison to several negative controls (FIGS. 2B-2D).

### Example 2: Single-Cell In Situ Analysis of RNA Modifications in Intact Tissues

Recent evidence has shown that RNA modifications are not just subtle structural modifiers, but also active gene regulators.³ In particular, mRNA modifications (e.g., N⁶-methyladenosine) impact almost every step of an RNA transcript's life cycle from birth to death, including splicing, nuclear export, storage, cellular localization, translation, and decay.³ It is still unknown, however, why mRNA modifications are non-stoichiometric. For a given gene, many modification sites are not 100% marked, and a number of RNA-de-modifying enzymes dynamically tune the status of modifications. New methods are required to determine what role these contrasting RNA modification states play in mRNA behavior, which is severely confounded by bulk sampling. It is also unknown how mRNA modifications affect the subcellular locations of mRNAs inside cells. Given the non-stoichiometric nature of mRNA modifications, there are at least two plausible hypotheses: within a single cell, mRNAs with different modification status have different spatiotemporal properties in order to precisely control the location and duration of protein production; or the apparent non-stoichiometry is a result of mixing different cell states and cell types, as previous measurements were all conducted with millions of cells. Finally, it is also unknown how the effects of mRNA modification vary between different cell types and states. Bulk epitranscriptomic sequencing results have revealed that different organs have distinct epitranscriptomic signatures, which change over different development and disease stages.³ Moreover, given the diversity of cell types within any given organ, a single-cell epitranscriptomic sequencing method is needed to reveal cell-type specific modification patterns. Dissecting this diversity will enable a deeper understanding of the epitranscriptomic pathway.

Current bulk epitranscriptomic sequencing methods have numerous limitations, including that they require millions of cells as input materials. This leads to loss of single-molecule stoichiometry and single-cell sensitivity. Furthermore, in applications where spatial relations are indispensable, such as developmental pathways and neuronal tissues, dissociated tissue samples are insufficient.⁵ Currently, imaging methods for RNA modifications can either only target one gene at a time, or detect all the modification sites as a sum without differentiating by gene identity. It is noteworthy that many modified RNAs are regulated as large gene groups. Thus, it is necessary to simultaneously measure at least hundreds of modified genes (ideally transcriptome-wide) for meaningful data analysis.

Overall, the methods described herein represents a transformative toolbox for single-cell epitranscriptomic profiling with spatial resolution. Such a system has broad applications in diverse biological processes. Given that RNA modifications are shared among all eukaryotic cells and RNA viruses that replicate inside cell nuclei, RNA modifications have been actively studied in cancer, immunology, RNA virology, etc. In addition, the spatial distribution of these modifications is likely important in fields including neuroscience, stem cell differentiation, and developmental biology. The methods described herein also have the potential to provide an integrative transcriptomic and epitranscriptomic spatial cell atlas of the brain that cannot be charted by existing approaches and new scientific knowledge on how RNA modifications regulate cell functions across different cell types and how single-cell events collectively impact tissue function. Besides m⁶A, there are other types of mRNA modifications (e.g., 1-methyladenosine, pseudouridine, etc.) that can be approached using similar strategies. Moreover, tRNAs have a plethora of diverse modifications.³ Increasing evidence indicates that they can also impact protein translation in response to various signals, stresses, and human diseases.^{3,5}

N⁶-methyladenosine (m⁶A) is the most prevalent internal modification present in the messenger RNA of all higher eukaryotes. This modification is non-stoichiometric and is installed by the m⁶A methyltransferase complex METTL3/14 (“writers”) whose deficiency is lethal in vertebrates; its presence is further dynamically regulated by m⁶A demethylases (ALKBH5/FTO; “erasers”) which impact development, fertility, and nutrient metabolism.³ A family of m⁶A-binding proteins, YTHDFs and YTHDCs, specifically recognize m⁶A-modified mRNAs and regulate mRNA splicing, export, translation, and degradation.^{12,13} Together, the m⁶A regulatory proteins endow gene expression with fast response and controllable protein production, essential during stem cell differentiation and animal development^{3,14}.

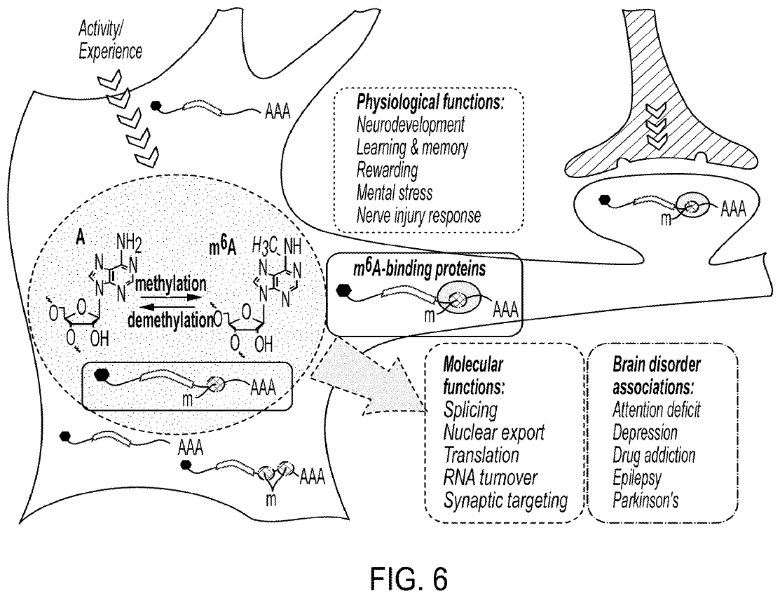

Bulk m⁶A-sequencing of rodents has revealed that m⁶A is prevalent in the whole brain transcriptome, modifying thousands of coding genes and hundreds of non-coding genes (FIG. 6).^{15,16} Studies of m⁶A pathway in animals have suggested that m⁶A modulates neuronal functions, including corticogenesis, dopaminergic signaling in the mouse midbrain, flight and locomotive behaviors in flies, neurogenesis in adult mice, and axon regeneration in mice.

Upregulation of m⁶A has been observed to occur with brain maturation, behavioral experience, and memory formation, suggesting a link between m⁶A accumulation and brain activity.⁶ Besides normal physiological processes, m⁶A abundance and genetic variants of m⁶A methyltransferases and demethylases have been associated with attention-deficit/hyperactivity disorder (ADHD), major depression disorder (MDD), addiction, epilepsy, and neurodegeneration.¹⁷

Despite these earlier discoveries of the link between m⁶A and brain function, however, there is still a vast knowledge gap between the association and the mechanism. Single-cell RNA sequencing has revealed hundreds of molecularly defined cell types¹⁸, while bulk m⁶A-seq masks the potential diversity of the m⁶A epitranscriptome at the single-cell level. For example, the m⁶A demethylase FTO regulates rewarding behavior via the dopamine-signaling reward circuit of meso-striato-prefrontal regions¹⁹, suggesting that m⁶A-dependent gene regulation could be more profound in defined neuronal cell types and biased towards specific neural circuits. In addition, many brain-related studies directly acknowledge and utilize the molecular mechanism discovered from m⁶A in cell cultures to explain the phenotype of mice. Such methodology could be problematic because the m⁶A pathway involves multiple enzymes and binding proteins and different combinations of them could lead to drastically different functional outcomes. Moreover, such in vitro experiments remove much of the context dependence of these cellular behaviors. Thus, it is highly desirable to directly study the m⁶A epitranscriptome in the animal model of interest to explain behavioral phenotypes, instead of using in vitro cell-culture models. In sum, it is clear that the lack of in situ m⁶A-seq in intact tissue is an important limitation, and the methods described herein address these issues.

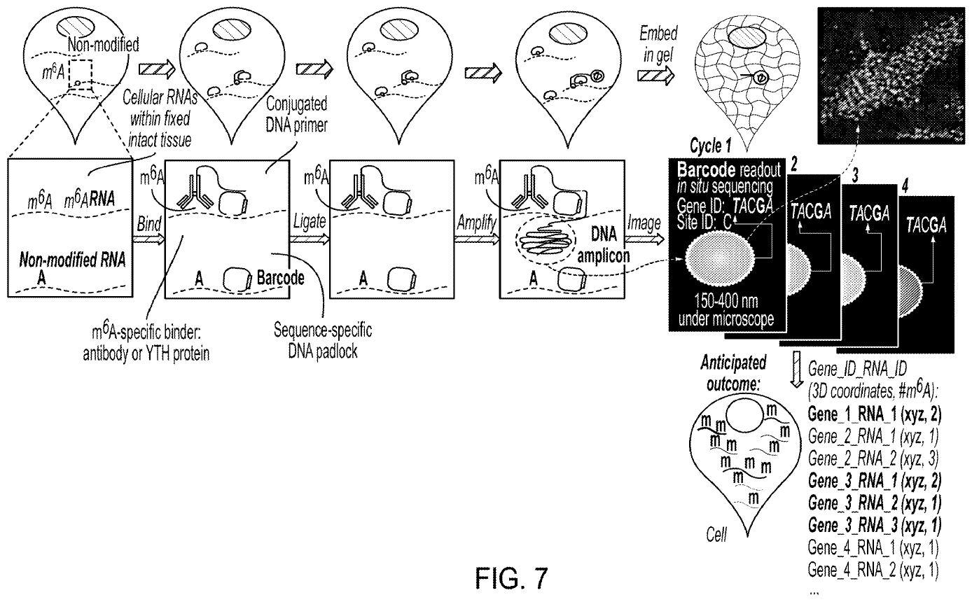

A method for 3D-m⁶A-seq was developed by utilizing m⁶A-specific binders, proximity amplification, and in situ RNA sequencing (FIG. 7): (1) m⁶A-binders (anti-m⁶A antibodies or biochemically purified m⁶A-specific YTH domain proteins^{12,13}) are conjugated with a DNA primer and used to stain samples of interest; (2) DNA padlock probes are then used to hybridize to the RNA sequence in proximity to m⁶A sites. Previously obtained m⁶A sites from bulk m⁶A-seq inform the design of these probes, a library of which allows for highly multiplexed targeting of thousands of m⁶A sites; (3) the padlock probes are circularized by an RNA-templated DNA ligase (e.g., SplintR); (4) the circularized DNA probes close to m⁶A sites are then amplified by rolling circle amplification (RCA, thousands of tandem repeats), while those near unmethylated sites cannot be amplified due to a lack of complementary primers attached to m⁶A binders needed for circularization; (5) each padlock probe contains a 5-base or longer barcode to encode gene identity (>1000 genes) and another 1-base barcode to encode m⁶A-site identity within the transcript (5 rounds of sequencing allows for up to 20 sites per transcript; >99% m⁶A modified genes have <20 sites per transcript); and (6) the gene identity and m⁶A-site identity are read out by 6 rounds of combinatorial SEDAL sequencing and 5 rounds sequential SEDAL sequencing¹¹, respectively. The outcome of such 3D-m⁶A-seq is a collection of single RNA molecules resolved with gene identity, 3D coordinates, and the number/location of m⁶A peaks. Such data can be directly visualized and analyzed to uncover the spatial distribution of gene expression with subcellular resolution (approximately 150-400 nm spatial resolution, depending on both the size of DNA amplicons and optical limits¹¹). Following cell-body staining (e.g., Nissl Staining) and cell segmentation, the RNAs can be attributed to each cell. The data can then be analyzed for simultaneous cell-type classification, evaluation of single-cell variation of gene expression, and m⁶A methylation status.

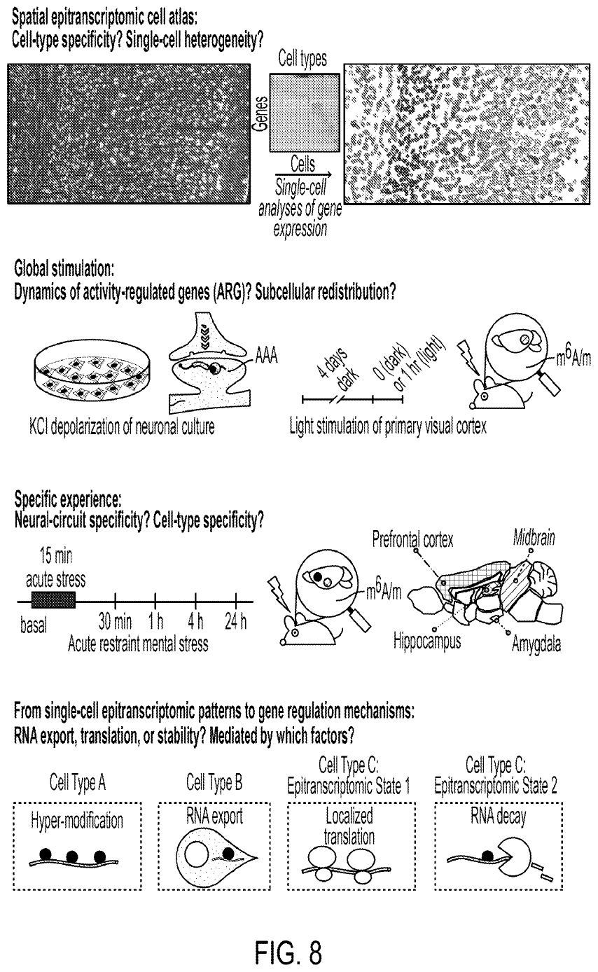

Previous studies have established a strong link between m6A and neural activity based on the evidence that: (1) activity-regulated genes (ARGs) and synaptic RNAs are m⁶A-modified; (2) disruption of the m⁶A pathway impairs neural plasticity and the formation of long-term memory; and (3) global m6A abundance changes as the animals are engaged with defined types of behaviors (fear, reward, stress etc.). The methods disclosed herein are useful for further dissecting the spatiotemporal patterns of m⁶A during global neural stimulation at subcellular resolution. 3D-m⁶A-seq can be applied to study the two most established biological systems for molecular-level investigation of neural activity: potassium chloride (KCI) depolarization of primary neuronal cell cultures and the dark/light conditioning of mice (FIG. 8)¹¹. Samples can be collected at a series of time points pre-and post-stimulation to trace the spatiotemporal dynamics and single-cell diversity of the m⁶A epitranscriptome in response to global neuron stimulation, evaluating the distribution of m⁶A RNAs in immediate-early genes (IEG) versus late-response genes (LRG).

After obtaining the single-cell resolved m⁶A patterns, the gene regulation mechanisms that shape such patterns can further be pinpointed. Two approaches to address this issue can be employed: (1) integrative analysis of single-cell m⁶A patterns together with the expression levels of m⁶A-related proteins (methyltransferase, demethylase, and binding proteins) to generate hypotheses on which protein is associated with changes in expression and m⁶A patterns; (2) cell-type specific gene perturbation (knockdown or overexpression) experiments to determine which factors are able to impact epitranscriptomic gene expression regulation in different cell types.

Existing bulk epitranscriptome sequencing methods require millions of cells as input materials, lacking single-molecule stoichiometry, single-cell sensitivity, and spatial resolution. Current imaging methods of RNA modification can only target one gene at a time or detect all the modifications sites as a sum without differentiating gene identity. 3D in situ sequencing of RNA modifications enables simultaneous and highly multiplexed mapping of the epitranscriptome with unprecedented resolution and precision: single-molecule stoichiometry with 3D coordinates for thousands of genes in intact biological tissues.

### Example 3: Three-Dimensional In Situ Profiling of Single-Cell Epitranscriptomics

The transcriptome serves as the pivotal mechanism that transmits information from the upstream genome to the downstream proteome, regulating numerous cellular processes. In recent years, single-cell RNA sequencing and spatial transcriptomic techniques have successfully mapped the transcriptomes of all kinds of biological samples, revealing intricate cell-to-cell heterogeneity in various systems. However, additional information beyond the RNA sequence is embedded in the transcriptome. This additional information, termed the epitranscriptome, namely includes the chemical modifications on mRNA, such as N⁶-methyladenosine (m⁶A), and the RNA-binding proteins (RBPs) associated with them, which are crucial in regulating almost every stage of the mRNA life cycle, ranging from mRNA synthesis, translocation, translation, and degradation. Knowing the spatially-resolved, single-cell profiles of the epitranscriptome is critical to a more complete understanding of how mRNAs are regulated in different cell types and states. Furthermore, little is known about the subcellular distribution of m⁶A and how the patterns are associated with the regulatory role of m⁶A. The present disclosure describes methods for in situ m⁶A mapping (m⁶A-map), a cutting-edge technology platform for three-dimensional (3D) in situ profiling of m⁶A at subcellular resolution. This method is used to generate imaging-based single-cell descriptions at the subcellular level with the goal of providing a fresh perspective on the spatial distribution of m⁶A. From a fundamental research perspective, this method can be generalized to aid the understanding of other RNA modifications, their associated RBPs, and their interactions. The application of m⁶A-map to intact tissues also facilitates identification of the causes and understanding of the development of diseases and disorders related to abnormal m⁶A levels.

To better understand different cell states and types, a variety of single-cell and spatial transcriptomic profiling methods have been developed to map the heterogeneities at the transcriptome level, yet these tools only depict cell states instead of revealing the mechanisms underlying the heterogeneous transcriptome. The epigenome and epitranscriptome therefore need to be interrogated at the single-cell level with spatial resolution, as they include additional information carried by nucleic acids that is responsible for regulating every stage of the mRNA life cycle.

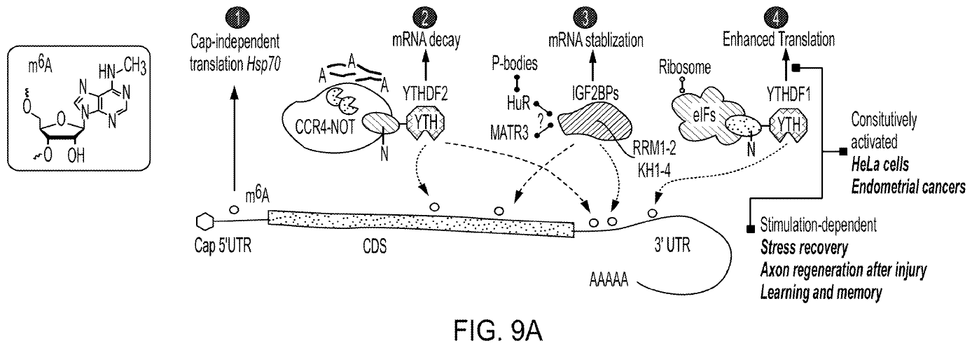

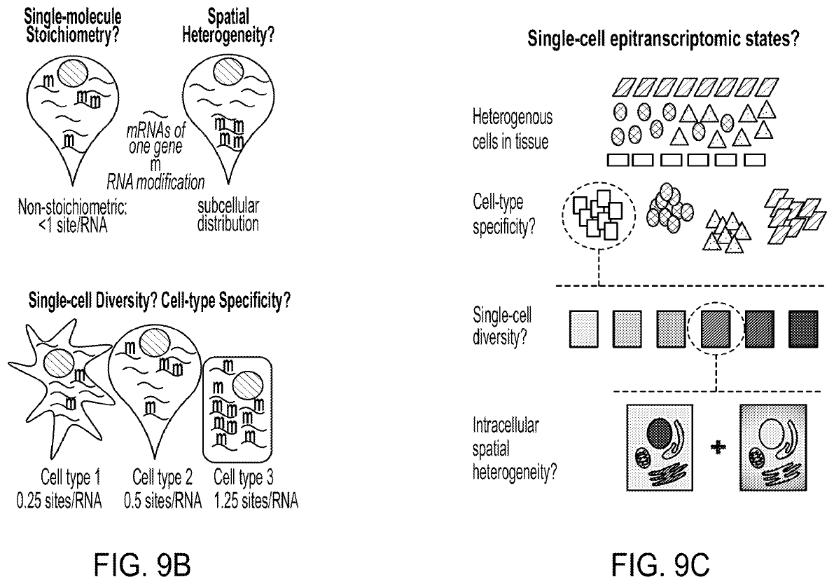

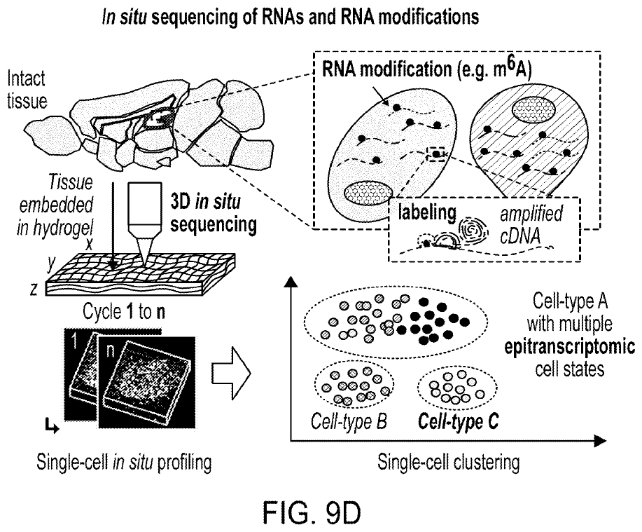

Among all epitranscriptomic modifications, m⁶A is the most abundant and arguably the most important modification on mRNA, which has been reported to regulate mRNA translation efficiency, stability, translocation, and splicing (FIG. 9A). Moreover, m⁶A is also implicated in many biological processes at tissue level, including learning and memory, cancer progression, and neurodegeneration. Thus, it is likely that different cells exhibit heterogeneous m⁶A methylomes and utilize m⁶A in different ways to regulate their transcriptome.

Nevertheless, the lack of single-cell resolution in epitranscriptomic studies has obscured the heterogeneity of m⁶A patterns in biological tissues and impairs the ability to dissect how m⁶A regulates gene expression across different cell types. So far, little is known about how mRNAs are regulated by m⁶A in different cell types and states and how the subcellular distribution patterns are associated with the regulatory role of m⁶A (FIG. 9B). For instance, what exactly the cell-type/state-specific m⁶A methylomes are, whether the same cell types can be divided into granular subtypes based on m⁶A methylome, whether m⁶A-mRNA and non-m⁶A-mRNAs exhibit different localizations at subcellular level and tissue level, how heterogeneous m⁶A affects different kinetics of RNA life cycle in different cell types, and many other such questions remain unanswered (FIG. 9C).

Previously, various tools have been developed to detect m⁶A modification qualitatively and quantitatively, yet none of them provide high throughput and single-cell, single-base, and spatial resolution at the same time. The present disclosure describes m⁶A-map (single-cell in situ mapping of m⁶A), a novel method that overcomes the aforementioned limitations of previous methods and resolves the m⁶A methylome at subcellular resolution (FIG. 9D).

### Method Design and Development in HeLa Cells (m⁶A-map v1)

First, two sets of three-part probes, each set comprising a primer and a 5′ phosphorylated, uniquely barcoded padlock probe, were in situ hybridized to the mRNA region beside each putative m⁶A site. To achieve sensitive and specific m⁶A detection, m⁶A recognition was linked with the recruitment of an oligonucleotide probe (the third probe in the set of probes). PAPG, a chimeric protein that specifically recognizes the Fc region of antibodies, is conjugated to the oligo (FIG. 10B). The oligo serves as a splint probe for in situ proximity ligation of padlock probes. In this way, only sites with m⁶A will have their corresponding padlock probes circularized. Additionally, to overcome autofluorescence and optical scattering, the circularized padlock probes were amplified using in situ rolling circle amplification (RCA). To preserve the locations of the amplicons and stabilize cellular structures during multiple rounds of sequencing and stripping, amino-allyl-dUTP was spiked in the RCA reaction so that the amplification products (amplicons) would be functionalized with primary amine groups, which were further acryloylated by methacrylic acid N-hydroxysuccinimide ester (MA-NHS) and embedded in a polyacrylamide hydrogel network. Finally, the barcodes on padlock probes were in situ sequenced, achieving multiplexity up to thousands of genes (FIG. 10A). Detection of single m⁶A sites (ACTB and MALAT1) in both cell culture and tissue has been demonstrated with a 15-30-fold signal enrichment in anti-m⁶A groups over non-specific IgG control groups (FIGS. 10C and 10E).

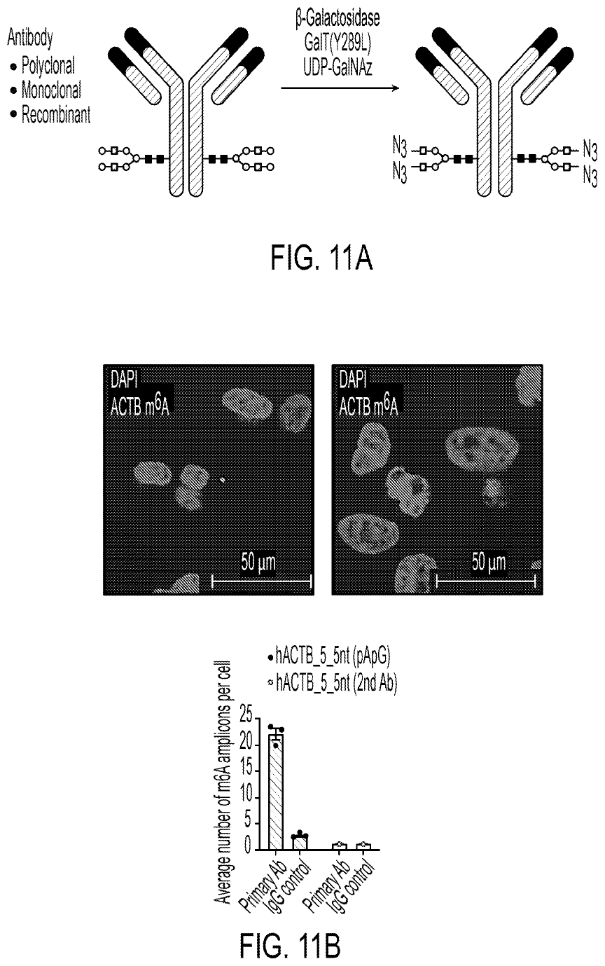

In the workflow described above, PAPG may be replaced with a secondary antibody. The secondary antibody can be conjugated to the oligo using, for example, the SiteClick antibody labeling kit (FIG. 11A). m⁶A-map v1 was tested with rabbit secondary antibody (FIG. 11B). An antibody-independent biotinylated-YTH-streptavidin-oligo detection scheme was tested as well (FIG. 10D).

The method can also be optimized in multiple ways. For example: (1) the optimal position of the probes was found to be 10 nt from m⁶A sites of interest; (2) rRNA blocking probes, which can hybridize to rRNA sequences containing m⁶A sites, can be added to the hybridization mixture to reduce m⁶A signals from rRNA m⁶A sites that are false positives; and (3) anchoring mRNAs to the hydrogel before hybridization followed by proteinase treatment can allow for better antibody diffusion into the sample (e.g., EDC that preferentially reacts with RNAs can be used to attach a polymerizable handle to the RNA).

### Method Design and Development in HeLa Cells (m⁶A-map v2)

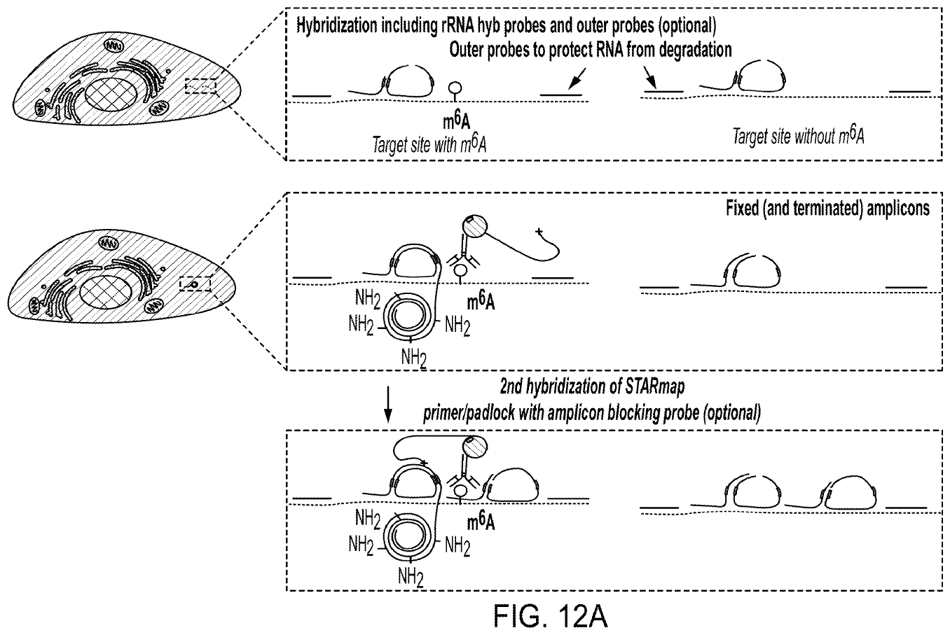

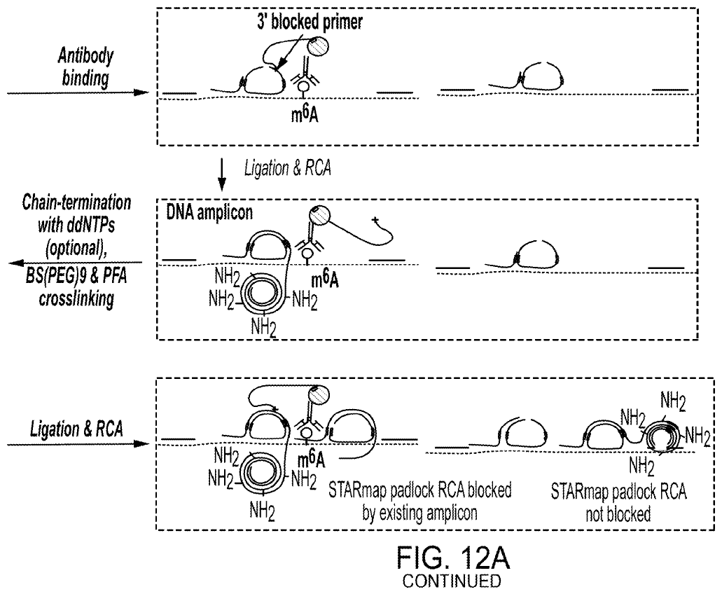

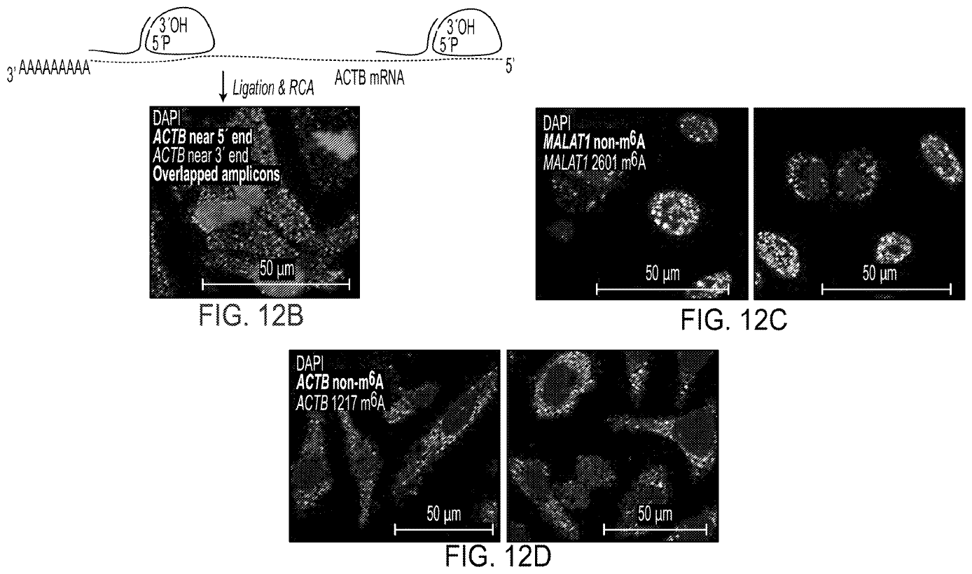

While m⁶A-map v1 can only detect m⁶A-modified RNA but cannot detect unmethylated RNA, it is very valuable to simultaneously detect both methylated and unmethylated RNA given the following considerations: (1) classification of cell states or cell types using marker gene expression; (2) estimation of m⁶A stoichiometry to distinguish whether a higher m⁶A signal from a certain gene is due to a higher expression level or a higher m⁶A fraction; (3) comparison between the differential localization of methylated RNA and unmethylated RNA; and (4) integration with other in situ sequencing data. An updated method, m⁶A-map v2, was devised to address this issue. The steps before the second hybridization are the same as m⁶A-map v1. After the first RCA, a second hybridization using STARmap probes is performed, which allows for the detection of non-m⁶A RNAs, followed by ligation and the second RCA (FIG. 12A). If there is already a DNA amplicon attached to the RNA, the second RCA will not be able to occur because the first DNA amplicon has taken up most of the available space (FIG. 12B). Both m⁶A and non-m⁶A RNAs can thereby be quantified separately, and the fraction of m⁶A RNAs can be calculated. Quantification of the m⁶A fraction at single m⁶A sites (ACTB and MALAT1) in cell culture was demonstrated (FIGS. 12C and 12D). The m⁶A fractions at different cell cycle phases were indeed different, and m⁶A RNAs and non-m⁶A RNAs showed distinct subcellular localization.

### Method Design and Development in HeLa Cells (RBP-RNA Mapping)

Apart from m⁶A, RBP-RNA interaction is another critical aspect of the epitranscriptome since most mRNA modifications achieve their functions via differential binding of various RBPs. Although multiple single-cell RBP-RNA interactome mapping methods have been developed, these methods are either tailored for detecting ribosome-mRNA interaction specifically or require transfection of exogenous gene constructs. Moreover, they cannot preserve the spatial location of cells and subcellular locations of RBP-RNA interactions.

In addition, m⁶A sometimes has contradictory functions depending on context, and the main reason is that it may be bound by different reader RBPs that trigger completely different downstream pathways. A multiplexed detection method would be ideal for interpretation. Therefore, an in situ multiplexed RBP-RNA mapping technology was developed, which includes transferring the information of oligo-conjugated antibodies to the padlock probe followed by in situ sequencing of transferred barcode information (FIG. 13A). The feasibility of single RBP-RNA mapping has already been demonstrated (FIG. 13B), and the next step is to multiplex this method (FIG. 13C). Such a method is useful for determining how epitranscriptome and RBP-RNA interaction contributes to cell-state/type-specific transcriptome regulation and tissue functions.

### 100-Gene Detection in HeLa Cells as a Proof-of-Concept Study

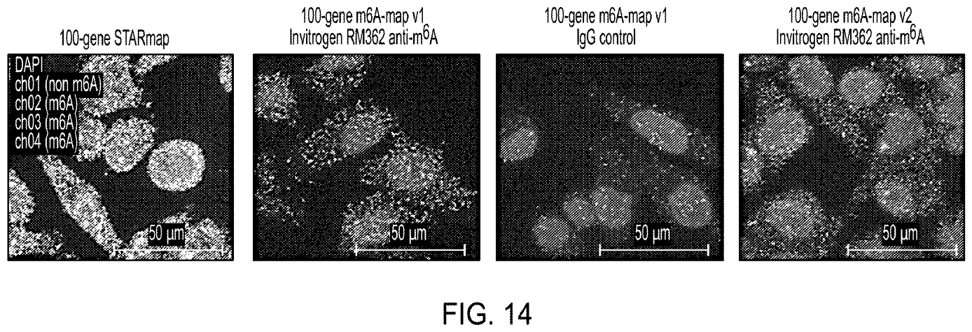

To further demonstrate that m⁶A-map can detect multiple m⁶A sites simultaneously in a high-throughput manner, a 100-gene detection was performed on Fluorescent Ubiquitination-based Cell Cycle Indicator (FUCCI) HeLa cells. Spatial transcriptomic profiling (STARmap) and m⁶A-map v1 were performed simultaneously. Antibodies from different vendors were tested in order to select the one with the highest detection efficiency. The nuclei were stained with DAPI, while the cell bodies were stained with Flamingo and the endoplasmic reticulum (ER) were stained with concanavalin A, which enabled visualization of probes for three moderately-expressed housekeeping genes (HMBS, MRPL19, and PGK1). Consistent anti-m⁶A signal enrichment over IgG control was observed (FIG. 14), further validating the signal-to-noise ratio (SNR) that was achieved in method optimization.

### Findings from m⁶A-Map v1 100-Gene Dataset

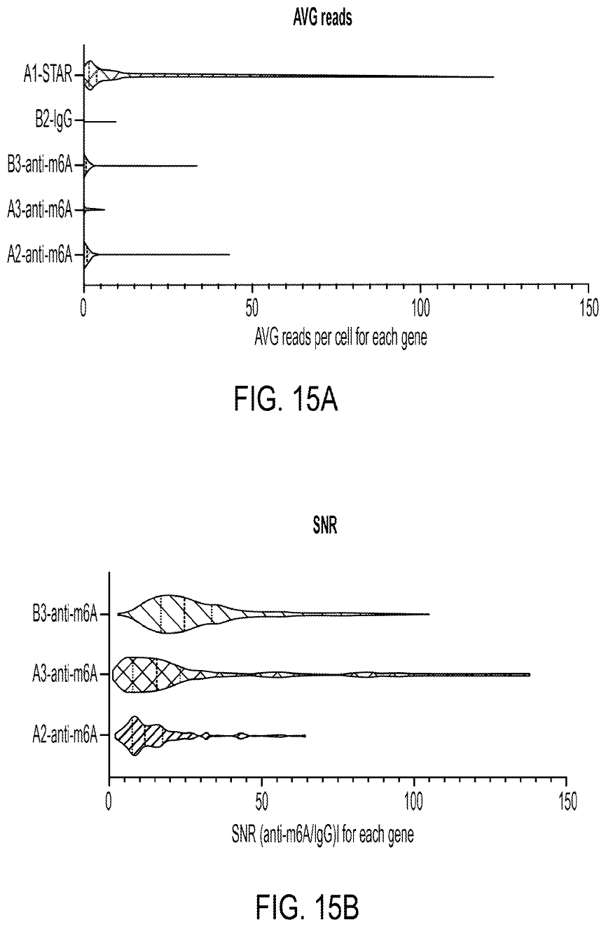

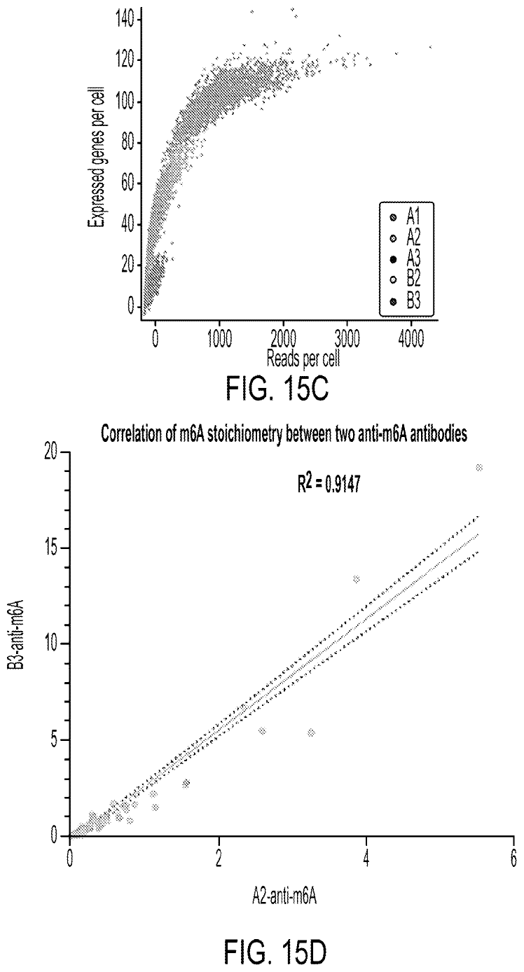

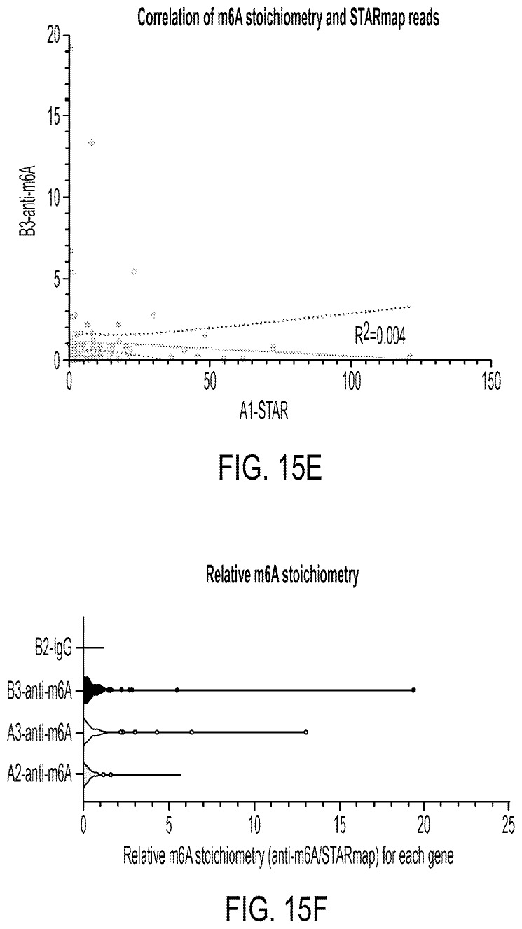

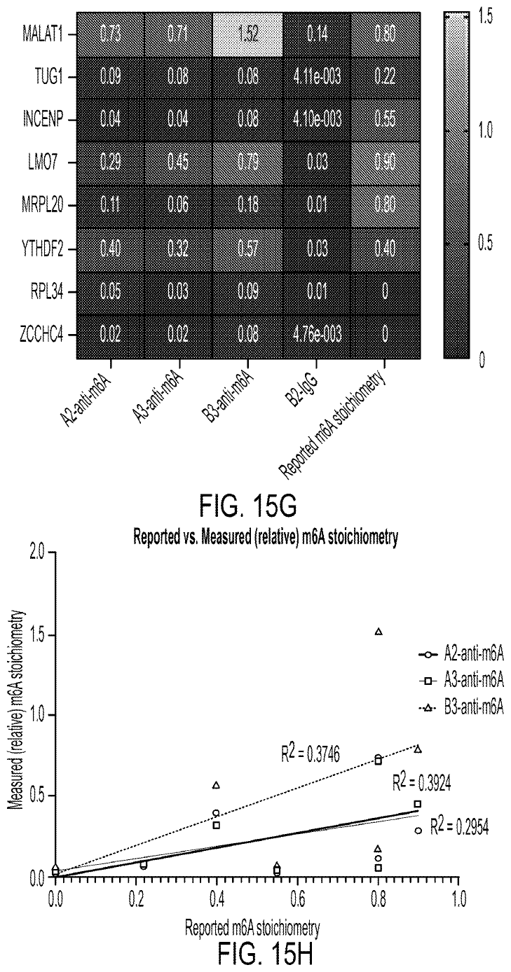

The in situ sequencing data and FUCCI imaging data of m⁶A-map v1 was preprocessed by deconvolution, spot finding, read assignment, cellular and subcellular segmentation, alignment, filtering, and normalization. The number of reads of m⁶A-map is lower than that in STARmap (FIGS. 15A and 15C). The Invitrogen RM362 anti-m⁶A antibody achieved the best signal-to-noise ratio (SNR), reaching an SNR of 25 for most of the genes (FIG. 15B). The correlation of measured m⁶A stoichiometry (reads in m⁶A-map/reads in STARmap) between two anti-m⁶A antibodies were well correlated, verifying the robustness of the method (FIG. 15D). The m⁶A stoichiometry of different genes had a slight negative correlation with their expression level, indicating that m⁶A mainly promotes mRNA decay (FIG. 15E). The method can be used to semi-quantitatively estimate the relative m⁶A stoichiometry, showing a positive correlation to literature-reported stoichiometry for some representative genes (FIGS. 15F and 15H).

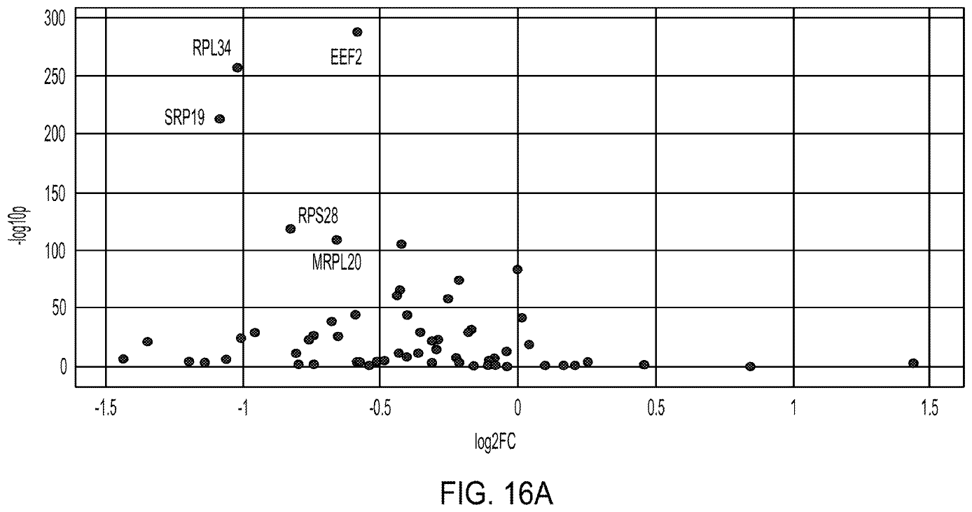

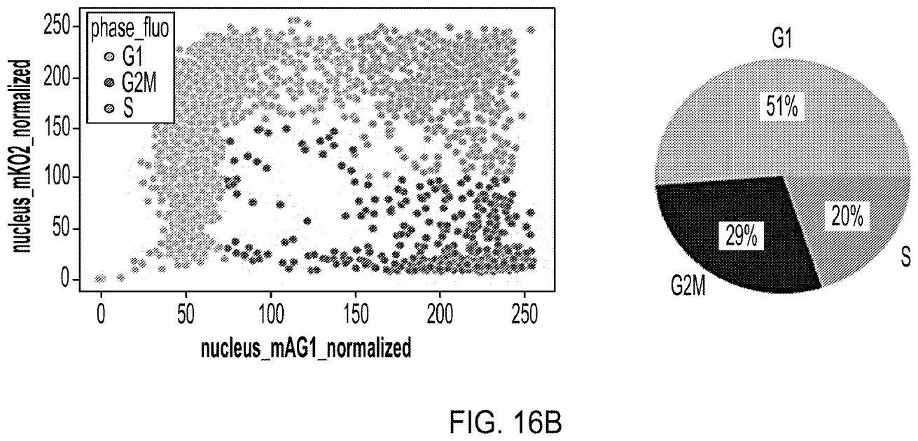

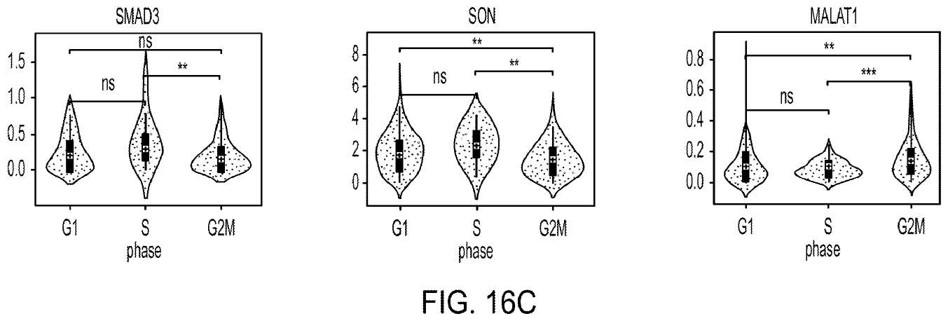

To benchmark and gain more biological insight into m⁶A biology, the subcellular distribution pattern of m⁶A vs. non-m A mRNAs was analyzed. It was found that mRNAs with more m⁶A deposition tend to localize to cytoplasm more readily, confirming the biological function of m⁶A promoting nuclear export (FIG. 16A). Genes that are strongly affected by m⁶A deposition include many genes related to translation, such as EEF2, SRP19, and MRPL20, indicating that m⁶A may be important for efficient production of translation-related proteins. YTHDC1-targeting mRNAs may exhibit a higher dependence on m⁶A for nuclear export.

Cell-cycle analyses were also carried out on the dataset. FUCCI fluorescent intensity was quantified for each cell to determine the cell cycle phases of different cells (FIG. 16B). The m⁶A stoichiometry was calculated for each gene in different cell cycle phases. Many genes were found to exhibit cell-cycle dependent m⁶A fluctuations. SMAD3, a gene related to transcription regulation, was shown to have higher m⁶A stoichiometry in G1 and S but lower in G2M, which agrees with the results in the literature (Fei, Q. et al., YTHDF2 promotes mitotic entry and is regulated by cell cycle mediators. PLoS Biol 18, e3000664 (2020)). It was also found that other genes, including SON, have a higher m⁶A stoichiometry in S and G1 but lower in G2M, and while some such as MALAT1 have a higher m⁶A stoichiometry in G2M and G1 but lower in S.

### Discussion

In summary, a novel strategy, m⁶A-map, was developed that can be used to measure hundreds or thousands of m⁶A loci in single cells with subcellular resolution and estimate the relative stoichiometry for each locus in a semi-quantitative manner. m⁶A-map can be used to discover brand new biological insights related to the epitranscriptome, such as whether different epitranscriptomic status affects the subcellular localization of RNA, how the epitranscriptome exhibits cell-to-cell heterogeneity, and whether m⁶A levels fluctuate in different cell cycle phases. The methods described herein are versatile tools for exploring m⁶A biology in situ for cell culture and tissue samples.

### REFERENCES

1. Zhu, C., Preissl, S. & Ren, B. Nat Methods 17, 11-14 (2020).
2. Wang X., Allen W., Wright M., Sylwestrak E., Samusik N., Vesuna S., Evans K., Liu C., Ramakrishnan C., Liu J., Nolan G.P., Bava F.A., Deisseroth K. Science (2018).
3. Roundtree, I.A. et al. Dynamic RNA modifications in gene expression regulation. Cell 169, 1187 (2017).
4. Jonkhout, N. et al. The RNA modification landscape in human disease. RNA 23, 1754 (2017).
5. Li, X. et al. Epitranscriptome sequencing technologies: decoding RNA modifications. Nature Methods 14, 23 (2017).
6. Widagdo, J. et al. The m⁶A-epitranscriptomic signature in neurobiology: from neurodevelopment to brain plasticity. Journal of Neurochemistry 147, 137 (2018).
7. Chen, K. H. et al. Spatially resolved, highly multiplexed RNA profiling in single cells. Science 348, 6090 (2015).
8. Shah, S. et al. seqFISH accurately detects transcripts in single cells and reveals robust spatial organization in the hippocampus. Neuron 94, 752 (2017).
9. Ke, R. et al. In situ sequencing for RNA analysis in preserved tissue and cells. Nature Methods 10, 857 (2013).
10. Lee, J. H. et al. Highly multiplexed subcellular RNA sequencing in situ. Science 343, 1360 (2014).
11. Wang, X. et al. Three-dimensional intact-tissue sequencing of single-cell transcriptional states. Science 361, eaat5691 (2018).
12. Wang, X. et al. N⁶-methyladenosine-dependent regulation of messenger RNA stability. Nature 505, 117 (2014).
13. Wang, X. et al. N⁶-methyladenosine modulates messenger RNA translation Efficiency. Cell 161, 1388 (2015).
14. Zhao, B.S. et al. m⁶A-dependent maternal mRNA clearance facilitates zebrafish maternal-to-zygotic transition. Nature 542, 475 (2017).
15. Dominissini, D. et al. The topology of the human and mouse m⁶A RNA methylomes revealed by m⁶A-seq. Nature 485, 201 (2012).
16. Meyer, K. D. et al. Comprehensive analysis of mRNA methylation reveals enrichment in 3′ UTRs and near stop codons. Cell 149, 1635 (2012).
17. McGuinness, D.H. et al. m⁶A RNA methylation: the implications for health and disease. Journal of Cancer Science and Clinical Oncology 105 (2014).
18. Fishell, G. & Heintz, N. The neuron identity problem: form meets function. Neuron 80, 602 (2013).
19. Hess, M.E. et al. The fat mass and obesity associated gene (Fto) regulates activity of the dopaminergic midbrain circuitry. Nature Neuroscience 16, 1042 (2013).
20. Chen, F. et al. Expansion Microscopy. Science 347, 543 (2015).
21. Cox, D.B.T. et al. RNA editing with CRISPR-Cas 13. Science 358, 1019 (2017).
22. Liu, N. et al. Probing N⁶-methyladenosine RNA modification status at single nucleotide resolution in mRNA and long non-coding RNA. RNA 19, 1848 (2013).
23. Engel, M. et al. The Role of m⁶A/m-RNA Methylation in Stress Response Regulation. Neuron 99, 389 (2018).

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A method for profiling epitranscriptomic RNA modifications in a cell, the method comprising:
a) contacting the cell with one or more sets of probes, wherein each set of probes comprises a first probe, a second probe, and a third probe wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an epitranscriptomic RNA modification and an oligonucleotide portion that is complementary to a portion of the first probe; and
iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell.

2. The method of claim 1, wherein the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), or 5-methylcytosine (m⁵C).

3. The method of claim 1, wherein the epitranscriptomic RNA modification is No-methyladenosine (m⁶A).

4. The method of any one of claims 1-3, wherein the portion of the second probe that recognizes the epitranscriptomic RNA modification comprises an agent that binds an antibody, or an antibody variant.

5. The method of any one of claims 1-4, wherein the portion of the second probe that recognizes the epitranscriptomic RNA modification comprises a protein.

6. The method of claim 5, wherein the protein comprises PAPG.

7. The method of claim 6 further comprising contacting the cell with an antibody that recognizes an epitranscriptomic RNA modification, wherein the antibody that recognizes an epitranscriptomic RNA modification is recognized and bound by PAPG.

8. The method of any one of claims 1-4, wherein the portion of the second probe that recognizes the epitranscriptomic RNA modification comprises an antibody, or an antibody variant.

9. The method of claim 8, wherein the antibody is a secondary antibody.

10. The method of claim 9 further comprising contacting the cell with a primary antibody that recognizes the epitranscriptomic RNA modification and is recognized by the secondary antibody of the second probe.

11. The method of claim 10, wherein the cell is contacted with the primary antibody prior to being contacted with the one or more pairs of probes.

12. The method of claim 10 or 11, wherein the primary antibody is an anti-m⁶A antibody, an anti-m¹A antibody, an anti-pseudouridine antibody, an anti-m⁶Am antibody, an anti-m⁷G antibody, an anti-ac⁴C antibody, an anti-Nm antibody, or an anti-m⁶C antibody.

13. The method of any one of claims 1-12, wherein the portion of the second probe that recognizes the epitranscriptomic RNA modification comprises an agent that binds the epitranscriptomic RNA modification directly.

14. The method of claim 13, wherein the agent that binds the epitranscriptomic RNA modification directly comprises a protein.

15. The method of claim 14, wherein the protein is an m⁶A-specific YTH domain protein.

16. The method of claim 13, wherein the agent that binds the epitranscriptomic RNA modification directly comprises an antibody, or an antibody variant.

17. The method of any one of claims 1-16, wherein the second probe further comprises a polymerization blocker.

18. The method of claim 17, wherein the polymerization blocker is located at the 3′ end of second probe.

19. The method of claim 17 or 18, wherein the polymerization blocker comprises an inverted nucleic acid residue.

20. The method of claim 19, wherein the inverted nucleic acid residue is an inverted thymine residue.

21. The method of any one of claims 1-20, wherein the oligonucleotide barcode sequence of the first probe is about 1-10, about 2-9, about 3-8, about 4-7, or about 5-6 nucleotides in length.

22. The method of any one of claims 1-21, wherein the oligonucleotide barcode of the first probe is about 1, about 2, about 3, about 4, about 5, about 6, about 7, about 8, about 9, or about 10 nucleotides in length.

23. The method of any one of claims 1-22, wherein the first and third oligonucleotide probes are complementary to different portions of the RNA of interest.

24. The method of any one of claims 1-23, wherein the first probe comprises the structure:
5′-[portion complementary to second probe]-[oligonucleotide barcode sequence]-[portion complementary to RNA of interest]-[portion complementary to third probe]-[portion complementary to second probe]-3′,
wherein each instance of]-[represents an optional linker.

25. The method of any one of claims 1-24, wherein the portion of the first probe that is complementary to a portion of the second probe is 4-20, 5-19, 6-18, 7-17, 8-16, 9-15, 10-14, or 11-13 nucleotides in length.

26. The method of any one of claims 1-25, wherein the portion of the first probe that is complementary to a portion of the second probe is 4, 5, 6, 7, 8, 9, 10, 11, 12, 13, 14, 15, 16, 17, 18, 19, or 20 nucleotides in length.

27. The method of any one of claims 1-26, wherein the portion of the first probe that is complementary to a portion of the second probe is split between the 5′ end and the 3′ end of the first probe.

28. The method of any one of claims 1-27, wherein the portion of the first probe that is complementary to a portion of the third probe is 5-15, 6-14, 7-13, 8-12, or 9-11 nucleotides in length.

29. The method of any one of claims 1-28, wherein the portion of the first probe that is complementary to a portion of the third probe is 5, 6, 7, 8, 9, 10, 11, 12, 13, 14, or 15 nucleotides in length.

30. The method of any one of claims 1-29, wherein the portion of the first probe that is complementary to the RNA of interest is 10-30, 11-29, 12-28, 13-27, 14-26, 15-25, 16-24, 17-23, 18-22, or 19-21 nucleotides in length.

31. The method of any one of claims 1-30, wherein the portion of the first probe that is complementary to the RNA of interest is 10, 11, 12, 13, 14, 15, 16, 17, 18, 19, 20, 21, 22, 23, 24, 25, 26, 27, 28, 29, or 30 nucleotides in length.

32. The method of any one of claims 1-31, wherein the second probe comprises the structure:
5′-[portion recognizing epitranscriptomic RNA modification]-[portion complementary to first probe]-3′, wherein ]-[represents an optional linker.

33. The method of any one of claims 1-32, wherein the portion of the second probe that is complementary to a portion of the first probe is 4-20, 5-19, 6-18, 7-17, 8-16, 9-15, 10-14, or 11-13 nucleotides in length.

34. The method of any one of claims 1-33, wherein the portion of the second probe that is complementary to a portion of the first probe is 4, 5, 6, 7, 8, 9, 10, 11, 12, 13, 14, 15, 16, 17, 18, 19, or 20 nucleotides in length.

35. The method of any one of claims 1-34, wherein the third probe comprises the structure:
5′-[portion complementary to RNA of interest]-[portion complementary to first probe]-3′, wherein]-[represents an optional linker.

36. The method of any one of claims 1-35, wherein the portion of the third probe that is complementary to the RNA of interest is 10-30, 11-29, 12-28, 13-27, 14-26, 15-25, 16-24, 17-23, 18-22, or 19-21 nucleotides in length.

37. The method of any one of claims 1-36, wherein the portion of the third probe that is complementary to the RNA of interest is 10, 11, 12, 13, 14, 15, 16, 17, 18, 19, 20, 21, 22, 23, 24, 25, 26, 27, 28, 29, or 30 nucleotides in length.

38. The method of any one of claims 1-37, wherein the portion of the third probe that is complementary to a portion of the first probe is 5-15, 6-14, 7-13, 8-12, or 9-11 nucleotides in length.

39. The method of any one of claims 1-38, wherein the portion of the third probe that is complementary to a portion of the first probe is 5, 6, 7, 8, 9, 10, 11, 12, 13, 14, or 15 nucleotides in length.

40. A method for profiling epitranscriptomic RNA modifications in a cell, the method comprising:
a) contacting the cell with one or more pairs of probes, wherein each pair of probes comprises a first probe and a second probe, wherein:
i) the first probe comprises an oligonucleotide portion that is complementary to the second probe, an oligonucleotide portion that is complementary to an RNA of interest, and an oligonucleotide barcode sequence; and
ii) the second probe comprises a portion that recognizes an epitranscriptomic RNA modification and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell.

41. The method of claim 40, wherein the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), or 5-methylcytosine (m⁵C).

42. The method of claim 41, wherein the epitranscriptomic RNA modification is N⁶-methyladenosine (m⁶A).

43. The method of any one of claims 40-42, wherein the portion of the second probe that recognizes the epitranscriptomic RNA modification comprises an agent that binds an antibody, or an antibody variant.

44. The method of any one of claims 40-43, wherein the portion of the second probe that recognizes the epitranscriptomic RNA modification comprises a protein.

45. The method of claim 44, wherein the protein comprises PAPG.

46. The method of claim 45 further comprising contacting the cell with an antibody that recognizes an epitranscriptomic RNA modification, wherein the antibody that recognizes an epitranscriptomic RNA modification is recognized and bound by PAPG.

47. The method of any one of claims 40-46, wherein the portion of the second probe that recognizes the epitranscriptomic RNA modification comprises an antibody, or an antibody variant.

48. The method of claim 47, wherein the antibody is a secondary antibody.

49. The method of claim 48 further comprising contacting the cell with a primary antibody that recognizes the epitranscriptomic RNA modification and is recognized by the secondary antibody of the second probe.

50. The method of claim 49, wherein the cell is contacted with the primary antibody prior to being contacted with the one or more pairs of probes.

51. The method of claim 49 or 50, wherein the primary antibody is an anti-m⁶A antibody, an anti-m¹A antibody, an anti-pseudouridine antibody, an anti-m⁶Am antibody, an anti-m⁷G antibody, an anti-ac⁴C antibody, an anti-Nm antibody, or an anti-m⁵C antibody.

52. The method of any one of claims 40-42, wherein the portion of the second probe that recognizes the epitranscriptomic RNA modification comprises an agent that binds the epitranscriptomic RNA modification directly.

53. The method of claim 52, wherein the agent that binds the epitranscriptomic RNA modification directly comprises a protein.

54. The method of claim 53, wherein the protein is an m⁶A-specific YTH domain protein.

55. The method of claim 52, wherein the agent that binds the epitranscriptomic RNA modification comprises an antibody, or an antibody variant.

56. The method of any one of claims 40-55, wherein the oligonucleotide portion of the first probe that is complementary to the second probe is about 3-20, about 4-19, about 5-18, about 6-17, about 7-16, about 8-15, about 9-14, about 10-13, or about 11-12 nucleotides in length.

57. The method of any one of claims 40-56, wherein the oligonucleotide portion of the first probe that is complementary to the second probe is about 3, about 4, about 5, about 6, about 7, about 8, about 9, about 10, about 11, about 12, about 13, about 14, about 15, about 16, about 17, about 18, about 19, or about 20 nucleotides in length.

58. The method of any one of claims 40-57, wherein the oligonucleotide portion of the first probe that is complementary to the second probe is split between the 5′ end and the 3′ end of the first probe.

59. The method of any one of claims 40-58, wherein the oligonucleotide portion of the first probe that is complementary to the RNA of interest is about 10-30, about 11-29, about 12-28, about 13-27, about 14-26, about 15-25, about 16-24, about 17-23, about 18-22, or about 19-21 nucleotides in length.

60. The method of any one of claims 40-59, wherein the oligonucleotide portion of the first probe that is complementary to the RNA of interest is about 10, about 11, about 12, about 13, about 14, about 15, about 16, about 17, about 18, about 19, about 20, about 21, about 22, about 23, about 24, about 25, about 26, about 27, about 28, about 29, about 30, or more than 30 nucleotides in length.

61. The method of any one of claims 40-60, wherein the oligonucleotide barcode sequence of the first probe is about 1-10, about 2-9, about 3-8, about 4-7, or about 5-6 nucleotides in length.

62. The method of any one of claims 40-61, wherein the oligonucleotide barcode sequence of the first probe is about 1, about 2, about 3, about 4, about 5, about 6, about 7, about 8, about 9, or about 10 nucleotides in length.

63. The method of any one of claims 40-62, wherein each portion of the first probe and the second probe is connected by an optional linker.

64. The method of claim 63, wherein each instance of the optional linker is independently 1, 2, 3, 4, 5, 6, 7, 8, 9, or 10 nucleotides long.

65. The method of any one of claims 40-64, wherein the first probe comprises the structure:
5′-[portion complementary to second probe]-[portion complementary to RNA of interest]-[barcode sequence]-[portion complementary to second probe]-3′;
5′-[portion complementary to second probe]-[barcode sequence]-[portion complementary to RNA of interest]-[portion complementary to second probe]-3′;
5′-[portion complementary to second probe]-[portion complementary to RNA of interest]-[barcode sequence]-3′;
5′-[portion complementary to second probe]-[barcode sequence]-[portion complementary to RNA of interest]-3′;
5′-[portion complementary to RNA of interest]-[barcode sequence]-[portion complementary to second probe]-3′; or
5′-[barcode sequence]-[portion complementary to RNA of interest]-[portion complementary to second probe]-3′,
wherein each instance of]-[comprises an optional linker.

66. The method of any one of claims 40-65, wherein the second probe comprises the structure:
5′-[portion recognizing epitranscriptomic RNA modification]-[portion complementary to first probe]-3′,
wherein each instance of]-[comprises an optional linker.

67. The method of any one of claims 40-66, wherein the oligonucleotide portions of the first probe and the second probe comprise DNA.

68. The method of any one of claims 1-67, wherein the RNA of interest is a messenger RNA (mRNA), a transfer RNA (tRNA), or a ribosomal RNA (rRNA).

69. The method of any one of claims 1-68, wherein epitranscriptomic RNA modifications are profiled in multiple cells simultaneously.

70. The method of claim 69, wherein epitranscriptomic RNA modifications are profiled in more than 10 cells, more than 20 cells, more than 50 cells, more than 100 cells, more than 200 cells, more than 300 cells, more than 400 cells, more than 500 cells, or more than 1000 cells simultaneously.

71. The method of claim 70, wherein the cells comprise a plurality of cell types.

72. The method of claim 71, wherein the cell types are selected from the group consisting of stem cells, progenitor cells, neuronal cells, astrocytes, dendritic cells, endothelial cells, microglia, oligodendrocytes, muscle cells, myocardial cells, mesenchymal cells, epithelial cells, immune cells, hepatic cells, smooth and skeletal muscle cells, hematopoietic cells, lymphocytes, monocytes, neutrophils, macrophages, natural killer cells, mast cells, adipocytes, and neurons.

73. The method of any one of claims 1-72, wherein the cell is present within an intact tissue.

74. The method of claim 73, wherein the intact tissue is a fixed tissue sample.

75. The method of claim 73 or 74, wherein the tissue is epithelial tissue, connective tissue, muscular tissue, or nervous tissue.

76. The method of any one of claims 1-75, wherein epitranscriptomic modification of more than 1, more than 2, more than 3, more than 4, more than 5, more than 10, more than 20, more than 30, more than 40, more than 50, more than 100, more than 200, more than 500, more than 1000, more than 2000, or more than 3000 RNAs is profiled simultaneously.

77. The method of any one of claims 1-76, wherein multiple different epitranscriptomic modifications are profiled simultaneously in the cell.

78. The method of any one of claims 1-77, wherein the oligonucleotide barcode sequence of the first probe is a gene specific sequence used to identify an RNA of interest.

79. The method of any one of claims 1-78, wherein the step of sequencing comprises performing sequencing with error-reduction by dynamic annealing and ligation (SEDAL).

80. The method of any one of claims 1-79, wherein the step of sequencing is repeated two, three, four, five, or more than five times.

81. The method of any one of claims 1-80, wherein the polymeric matrix is a hydrogel.

82. The method of claim 81, wherein the hydrogel is a polyvinyl alcohol hydrogel, a polyethylene glycol hydrogel, a polyacrylate hydrogel, or a polyacrylamide hydrogel.

83. The method of any one of claims 1-82, wherein the step of performing rolling circle amplification further comprises providing amine-modified nucleotides, wherein the amine-modified nucleotides are incorporated into the one or more concatenated amplicons.

84. The method of claim 83, wherein the step of embedding the one or more concatenated amplicons in the polymeric matrix comprises reacting the amine-modified nucleotides of the one or more amplicons with acrylic acid N-hydroxysuccinimide ester and co-polymerizing the one or more concatenated amplicons and the polymeric matrix.

85. The method of any one of claims 1-84 further comprising profiling additional molecules within the cell.

86. The method of claim 85, wherein the additional molecules are unmodified RNAs, DNAs, proteins, carbohydrates, or lipids.

87. The method of claim 85 or 86, wherein the additional molecules are unmodified RNAs.

88. The method of claim 87, wherein the unmodified RNAs are profiled by:
a) contacting the cell with one or more pairs of probes, wherein each pair of probes comprises a first probe and a second probe, wherein:
i) the first probe comprises an oligonucleotide portion complementary to a portion of the second probe, an oligonucleotide barcode sequence, and an oligonucleotide portion complementary to an unmodified RNA of interest; and
ii) the second probe comprises a portion that is complementary to the unmodified RNA of interest and a portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each unmodified RNA of interest in the cell.

89. The method of any one of claims 1-88 further comprising determining the cell type of the profiled cell by comparing the epitranscriptomic RNA modification profile of a cell to reference data comprising epitranscriptomic RNA modification profiles of various cell types.

90. The method of any one of claims 1-89 further comprising overexpressing or knocking out one or more genes in the cell to determine whether the one or more genes are involved in epitranscriptomic modification of the RNA of interest.

91. The method of any one of claims 1-90 further comprising repeating steps (a)-(e) at multiple time points to profile epitranscriptomic RNA modification in the cell over time.

92. A method for profiling interactions between an RNA-binding protein and one or more RNAs of interest in a cell, the method comprising:
a) contacting the cell with one or more sets of probes, wherein each set of probes comprises a first probe, a second probe, and a third probe wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an RNA-binding protein and an oligonucleotide portion that is complementary to a portion of the first probe; and
iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each RNA of interest bound by an RNA-binding protein in the cell.

93. The method of claim 92, wherein the RNA-binding protein comprises a YTH family protein (e.g., YTHDF1, YTHDF2, YTHDF3, YTHDC1, or YTHDC2), an IGF2BP family protein (e.g., IGF2BP1, IGF2BP2, or IGF2BP3), or FMR1.

94. The method of claim 92 or 93, wherein the portion of the second probe that recognizes the RNA-binding protein comprises an agent that binds an antibody, or an antibody variant.

95. The method of any one of claims 92-94, wherein the portion of the second probe that recognizes the RNA-binding protein comprises a protein.

96. The method of claim 95 further comprising contacting the cell with an antibody that recognizes an RNA-binding protein, wherein the antibody that recognizes an RNA-binding protein is recognized and bound by the protein.

97. The method of any one of claims 92-94, wherein the portion of the second probe that recognizes the RNA-binding protein comprises an antibody, or an antibody variant.

98. The method of claim 97, wherein the antibody is a secondary antibody.

99. The method of claim 98 further comprising contacting the cell with a primary antibody that recognizes the RNA-binding protein and is recognized by the secondary antibody of the second probe.

100. The method of claim 99, wherein the cell is contacted with the primary antibody prior to being contacted with the one or more pairs of probes.

101. The method of any one of claims 92-100, wherein the portion of the second probe that recognizes the RNA-binding protein comprises an agent that binds the RNA-binding protein directly.

102. The method of claim 101, wherein the agent that binds the RNA-binding protein directly comprises a protein.

103. The method of claim 101, wherein the agent that binds the RNA-binding protein directly comprises an antibody, or an antibody variant.

104. The method of any one of claims 92-103, wherein the second probe further comprises a polymerization blocker.

105. The method of claim 104, wherein the polymerization blocker is located at the 3′ end of second probe.

106. The method of claim 104 or 105, wherein the polymerization blocker comprises an inverted nucleic acid residue.

107. The method of claim 106, wherein the inverted nucleic acid residue is an inverted thymine residue.

108. The method of any one of claims 92-107, wherein the oligonucleotide barcode sequence of the first probe is about 1-10, about 2-9, about 3-8, about 4-7, or about 5-6 nucleotides in length.

109. The method of any one of claims 92-108, wherein the oligonucleotide barcode of the first probe is about 1, about 2, about 3, about 4, about 5, about 6, about 7, about 8, about 9, or about 10 nucleotides in length.

110. The method of any one of claims 92-109, wherein the first and third oligonucleotide probes are complementary to different portions of the RNA of interest.

111. The method of any one of claims 92-110, wherein the first probe comprises the structure:
5′-[portion complementary to second probe]-[oligonucleotide barcode sequence]-[portion complementary to RNA of interest]-[portion complementary to third probe]-[portion complementary to second probe]-3′, wherein each instance of]-[comprises an optional linker.

112. The method of any one of claims 92-111, wherein the portion of the first probe that is complementary to a portion of the second probe is 4-20, 5-19, 6-18, 7-17, 8-16, 9-15, 10-14, or 11-13 nucleotides in length.

113. The method of any one of claims 92-112, wherein the portion of the first probe that is complementary to a portion of the second probe is 4, 5, 6, 7, 8, 9, 10, 11, 12, 13, 14, 15, 16, 17, 18, 19, or 20 nucleotides in length.

114. The method of any one of claims 92-113, wherein the portion of the first probe that is complementary to a portion of the second probe is split between the 5′ end and the 3′ end of the first probe.

115. The method of any one of claims 92-114, wherein the portion of the first probe that is complementary to a portion of the third probe is 5-15, 6-14, 7-13, 8-12, or 9-11 nucleotides in length.

116. The method of any one of claims 92-115, wherein the portion of the first probe that is complementary to a portion of the third probe is 5, 6, 7, 8, 9, 10, 11, 12, 13, 14, or 15 nucleotides in length.

117. The method of any one of claims 92-116, wherein the portion of the first probe that is complementary to the RNA of interest is 10-30, 11-29, 12-28, 13-27, 14-26, 15-25, 16-24, 17-23, 18-22, or 19-21 nucleotides in length.

118. The method of any one of claims 92-117, wherein the portion of the first probe that is complementary to the RNA of interest is 10, 11, 12, 13, 14, 15, 16, 17, 18, 19, 20, 21, 22, 23, 24, 25, 26, 27, 28, 29, or 30 nucleotides in length.

119. The method of any one of claims 92-118, wherein the second probe comprises the structure:
5′-[portion recognizing RNA-binding protein]-[portion complementary to first probe]-3′, wherein]-[comprises an optional linker.

120. The method of any one of claims 92-119, wherein the portion of the second probe that is complementary to a portion of the first probe is 4-20, 5-19, 6-18, 7-17, 8-16, 9-15, 10-14, or 11-13 nucleotides in length.

121. The method of any one of claims 92-120, wherein the portion of the second probe that is complementary to a portion of the first probe is 4, 5, 6, 7, 8, 9, 10, 11, 12, 13, 14, 15, 16, 17, 18, 19, or 20 nucleotides in length.

122. The method of any one of claims 92-121, wherein the third probe comprises the structure:
5′-[portion complementary to RNA of interest]-[portion complementary to first probe]-3′, wherein ]-[comprises an optional linker.

123. The method of any one of claims 92-122, wherein the portion of the third probe that is complementary to the RNA of interest is 10-30, 11-29, 12-28, 13-27, 14-26, 15-25, 16-24, 17-23, 18-22, or 19-21 nucleotides in length.

124. The method of any one of claims 92-123, wherein the portion of the third probe that is complementary to the RNA of interest is 10, 11, 12, 13, 14, 15, 16, 17, 18, 19, 20, 21, 22, 23, 24, 25, 26, 27, 28, 29, or 30 nucleotides in length.

125. The method of any one of claims 92-124, wherein the portion of the third probe that is complementary to a portion of the first probe is 5-15, 6-14, 7-13, 8-12, or 9-11 nucleotides in length.

126. The method of any one of claims 92-125, wherein the portion of the third probe that is complementary to a portion of the first probe is 5, 6, 7, 8, 9, 10, 11, 12, 13, 14, or 15 nucleotides in length.

127. The method of any one of claims 92-126, wherein the RNA of interest is a messenger RNA (mRNA), a transfer RNA (tRNA), or a ribosomal RNA (rRNA).

128. The method of any one of claims 92-127, wherein interactions between RNA-binding proteins and RNAs of interest are profiled in multiple cells simultaneously.

129. The method of claim 128, wherein interactions between RNA-binding proteins and RNAs of interest are profiled in more than 10 cells, more than 20 cells, more than 50 cells, more than 100 cells, more than 200 cells, more than 300 cells, more than 400 cells, more than 500 cells, or more than 1000 cells simultaneously.

130. The method of claim 129, wherein the cells comprise a plurality of cell types.

131. The method of claim 130, wherein the cell types are selected from the group consisting of stem cells, progenitor cells, neuronal cells, astrocytes, dendritic cells, endothelial cells, microglia, oligodendrocytes, muscle cells, myocardial cells, mesenchymal cells, epithelial cells, immune cells, hepatic cells, smooth and skeletal muscle cells, hematopoietic cells, lymphocytes, monocytes, neutrophils, macrophages, natural killer cells, mast cells, adipocytes, and neurons.

132. The method of any one of claims 92-131, wherein the cell is present within an intact tissue.

133. The method of claim 132, wherein the intact tissue is a fixed tissue sample.

134. The method of claim 132 or 133, wherein the tissue is epithelial tissue, connective tissue, muscular tissue, or nervous tissue.

135. The method of any one of claims 92-134, wherein interactions between RNA-binding proteins and more than 1, more than 2, more than 3, more than 4, more than 5, more than 10, more than 20, more than 30, more than 40, more than 50, more than 100, more than 200, more than 500, more than 1000, more than 2000, or more than 3000 RNAs of interest are profiled simultaneously.

136. The method of any one of claims 92-135, wherein multiple different interactions between RNA-binding proteins and RNAs of interest are profiled simultaneously in the cell.

137. The method of any one of claims 92-136, wherein the oligonucleotide barcode sequence of the first probe is a gene specific sequence used to identify an RNA of interest.

138. The method of any one of claims 92-137, wherein the step of sequencing comprises performing sequencing with error-reduction by dynamic annealing and ligation (SEDAL).

139. The method of any one of claims 92-138, wherein the step of sequencing is repeated two, three, four, five, or more than five times.

140. The method of any one of claims 92-139, wherein the polymeric matrix is a hydrogel.

141. The method of claim 140, wherein the hydrogel is a polyvinyl alcohol hydrogel, a polyethylene glycol hydrogel, a polyacrylate hydrogel, or a polyacrylamide hydrogel.

142. The method of any one of claims 92-141, wherein the step of performing rolling circle amplification further comprises providing amine-modified nucleotides, wherein the amine-modified nucleotides are incorporated into the one or more concatenated amplicons.

143. The method of claim 142, wherein the step of embedding the one or more concatenated amplicons in the polymeric matrix comprises reacting the amine-modified nucleotides of the one or more amplicons with acrylic acid N-hydroxysuccinimide ester and co-polymerizing the one or more concatenated amplicons and the polymeric matrix.

144. The method of any one of claims 92-143 further comprising profiling additional molecules within the cell.

145. The method of claim 144, wherein the additional molecules are RNAs, DNAs, proteins, carbohydrates, or lipids.

146. The method of any one of claims 92-145 further comprising determining the cell type of the profiled cell by comparing the RNA-RBP interaction profile of a cell to reference data comprising RNA-RBP profiles of various cell types.

147. A method for diagnosing a disease or disorder in a subject, the method comprising:
a) contacting a cell taken from a subject with one or more sets of probes, wherein each set of probes comprises a first probe, a second probe, and a third probe wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an epitranscriptomic RNA modification and an oligonucleotide portion that is complementary to a portion of the first probe; and
iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell;
wherein a difference in the profile of epitranscriptomic RNA modifications in the cell relative to one or more non-diseased cells indicates that the subject has the disease or disorder.

148. A method for diagnosing a disease or disorder in a subject, the method comprising:
a) contacting a cell taken from a subject with one or more pairs of probes, wherein each pair of probes comprises a first probe and a second probe, wherein:
i) the first probe comprises an oligonucleotide portion that is complementary to the second probe, an oligonucleotide portion that is complementary to an RNA of interest, and an oligonucleotide barcode sequence; and
ii) the second probe comprises a portion that recognizes an epitranscriptomic RNA modification and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell;
wherein a difference in the profile of epitranscriptomic RNA modifications in the cell relative to one or more non-diseased cells indicates that the subject has the disease or disorder.

149. A method for diagnosing a disease or disorder in a subject, the method comprising:
a) contacting a cell taken from a subject with one or more sets of probes, wherein each set of probes comprises a first probe, a second probe, and a third probe wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an RNA-binding protein and an oligonucleotide portion that is complementary to a portion of the first probe; and
iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each RNA of interest bound by an RNA-binding protein in the cell;
wherein a difference in the profile of interactions between RNA and RNA-binding proteins in the cell relative to one or more non-diseased cells indicates that the subject has the disease or disorder.

150. The method of any one of claims 147-149, wherein the epitranscriptomic RNA modifications in one or more non-diseased cells, or the interactions between RNAs and RNA-binding proteins in one or more non-diseased cells, are profiled as a control experiment alongside the cell taken from the subject.

151. The method of any one of claims 147-149, wherein the profile of epitranscriptomic RNA modifications in one or more non-diseased cells, or the profile of interactions between RNAs and RNA-binding proteins in one or more non-diseased cells, comprises reference data.

152. The method of claim 147 or 148, wherein the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), or 5-methylcytosine (m⁵C).

153. The method of claim 147 or 148, wherein the epitranscriptomic RNA modification is N⁶-methyladenosine (m⁶A).

154. The method of claim 149, wherein the RNA-binding protein is a YTH family protein (e.g., YTHDF1, YTHDF2, YTHDF3, YTHDC1, or YTHDC2), an IGF2BP family protein (e.g., IGF2BP1, IGF2BP2, or IGF2BP3), or FMR1.

155. The method of any one of claims 147-154, wherein the disease or disorder is a genetic disease, a proliferative disease, an inflammatory disease, an autoimmune disease, a liver disease, a spleen disease, a lung disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, a central nervous system (CNS) disorder, or a cardiovascular disease.

156. The method of any one of claims 147-155, wherein the cell is present in a tissue.

157. The method of claim 156, wherein the tissue is epithelial tissue, connective tissue, muscular tissue, or nervous tissue.

158. The method of claim 156 or 157, wherein the tissue is a tissue sample taken from a subject.

159. The method of claim 158, wherein the subject is a non-human experimental animal.

160. The method of claim 158, wherein the subject is a human.

161. A method for screening for an agent capable of modulating epitranscriptomic modification of one or more RNAs, the method comprising:
a) contacting a cell that is being treated with or has been treated with a candidate agent with one or more sets of probes, wherein each set of probes comprises a first probe, a second probe, and a third probe wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an epitranscriptomic RNA modification and an oligonucleotide portion that is complementary to a portion of the first probe; and
iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell;
wherein a difference in the profile of epitranscriptomic RNA modifications in the cell in the presence of the candidate agent relative to in the absence of the candidate agent indicates that the candidate agent modulates epitranscriptomic RNA modification.

162. A method for screening for an agent capable of modulating epitranscriptomic modification of one or more RNAs, the method comprising:
a) contacting a cell that is being treated with or has been treated with a candidate agent with one or more pairs of probes, wherein each pair of probes comprises a first probe and a second probe, wherein:
i) the first probe comprises an oligonucleotide portion that is complementary to the second probe, an oligonucleotide portion that is complementary to an RNA of interest, and an oligonucleotide barcode sequence; and
ii) the second probe comprises a portion that recognizes an epitranscriptomic RNA modification and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each epitranscriptomically modified RNA of interest in the cell;
wherein a difference in the profile of epitranscriptomic RNA modifications in the cell in the presence of the candidate agent relative to in the absence of the candidate agent indicates that the candidate agent modulates epitranscriptomic RNA modification.

163. A method for screening for an agent capable of modulating interactions between RNA and RNA-binding proteins, the method comprising:
a) contacting a cell taken from a subject with one or more sets of probes, wherein each set of probes comprises a first probe, a second probe, and a third probe wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an RNA-binding protein and an oligonucleotide portion that is complementary to a portion of the first probe; and
iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix; and
e) sequencing the concatenated amplicons, or a portion thereof, embedded in the polymeric matrix to determine the identity and location of each RNA of interest bound by an RNA-binding protein in the cell;
wherein a difference in the profile of interactions between RNA and RNA-binding proteins in the presence of the candidate agent relative to in the absence of the candidate agent indicates that the candidate agent modulates interactions between RNA and RNA-binding proteins.

164. The method of claim 161 or 162, wherein the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N4-acetylcytidine (ac⁴C), 2′-O-methylation (Nm), or 5-methylcytosine (m⁵C).

165. The method of claim 161 or 162, wherein the epitranscriptomic RNA modification is N⁶-methyladenosine (m⁶A).

166. The method of claim 163, wherein the RNA-binding protein is a YTH family protein (e.g., YTHDF1, YTHDF2, YTHDF3, YTHDC1, or YTHDC2), an IGF2BP family protein (e.g., IGF2BP1, IGF2BP2, or IGF2BP3), or FMR1.

167. The method of any one of claims 161-166, wherein the candidate agent is a small molecule, a protein, a peptide, a nucleic acid, a lipid, or a carbohydrate.

168. The method of any one or claims 161-167, wherein the candidate agent is a known drug or an FDA-approved drug.

169. The method of claim 167 or 168, wherein the protein is an antibody, or an antibody variant.

170. The method of claim 167 or 168, wherein the nucleic acid is an mRNA, an antisense RNA, an miRNA, an siRNA, an RNA aptamer, a double stranded RNA (dsRNA), a short hairpin RNA (shRNA), or an antisense oligonucleotide (ASO).

171. The method of any one of claims 161-170, wherein modulating epitranscriptomic modification of one or more RNAs is associated with reducing, relieving, or eliminating the symptoms of a disease or disorder.

172. The method of claim 171, wherein the disease or disorder is a genetic disease, a proliferative disease, an inflammatory disease, an autoimmune disease, a liver disease, a spleen disease, a lung disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, a central nervous system (CNS) disorder, or a cardiovascular disease.

173. A method for treating a disease or disorder in a subject, the method comprising:
a) contacting a cell taken from a subject with one or more sets of probes, wherein each set of probes comprises a first probe, a second probe, and a third probe wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an epitranscriptomic RNA modification and an oligonucleotide portion that is complementary to a portion of the first probe; and
iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix;
e) sequencing the concatenated amplicons, or a portion thereof, embedded in the polymeric matrix to determine the identity and location of each epitranscriptomically modified RNA of interest in the cell; and
f) administering a treatment for the disease or disorder to the subject if a difference in the profile of epitranscriptomic RNA modifications in the cell relative to one or more non-diseased cells is observed.

174. A method for treating a disease or disorder in a subject, the method comprising:
a) contacting a cell taken from the subject with one or more pairs of probes, wherein each pair of probes comprises a first probe and a second probe, wherein:
i) the first probe comprises an oligonucleotide portion that is complementary to the second probe, an oligonucleotide portion that is complementary to an RNA of interest, and an oligonucleotide barcode sequence; and
ii) the second probe comprises a portion that recognizes an epitranscriptomic RNA modification and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the second probe as a primer to produce one or more concatenated amplicons;
d) embedding the one or more concatenated amplicons in a polymeric matrix;
e) sequencing the concatenated amplicons, or a portion thereof, embedded in the polymeric matrix to determine the identity and location of each epitranscriptomically modified RNA of interest in the cell; and
f) administering a treatment for the disease or disorder to the subject if a difference in the profile of epitranscriptomic RNA modifications in the cell relative to one or more non-diseased cells is observed.

175. A method for treating a disease or disorder in a subject, the method comprising:
a) contacting a cell taken from a subject with one or more sets of probes, wherein each set of probes comprises a first probe, a second probe, and a third probe wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an RNA-binding protein and an oligonucleotide portion that is complementary to a portion of the first probe; and
iii) the third probe comprises an oligonucleotide portion that is complementary to the RNA of interest and an oligonucleotide portion that is complementary to a portion of the first probe;
b) ligating the 5′ end and the 3′ end of the first probe together to produce a circular oligonucleotide;
c) performing rolling circle amplification to amplify the circular oligonucleotide using the third probe as a primer to produce one or more concatenated amplicons;
d) embedding the one or more concatenated amplicons in a polymeric matrix;
e) sequencing the concatenated amplicons, or a portion thereof, embedded in the polymeric matrix to determine the identity and location of each RNA of interest bound by an RNA-binding protein in the cell; and
f) administering a treatment for the disease or disorder to the subject if a difference in the profile of interactions between RNA and RNA-binding proteins in the cell relative to one or more non-diseased cells is observed.

176. The method of any one of claims 173-175, wherein epitranscriptomic modification of one or more RNAs in one or more non-diseased cells, or the interactions between RNAs and RNA-binding proteins in one or more non-diseased cells, is profiled simultaneously as a control experiment.

177. The method of any one of claims 173-175, wherein the epitranscriptomic modification profile of one or more RNAs in one or more non-diseased cells, or the profile of interactions between RNAs and RNA-binding proteins in one or more non-diseased cells, comprises reference data.

178. The method of any one of claims 173-177, wherein the treatment comprises administering a therapeutic agent, surgery, or radiation therapy.

179. The method of any one of claims 173-178, wherein the therapeutic agent is a small molecule, a protein, a peptide, a nucleic acid, a lipid, or a carbohydrate.

180. The method of any one of claims 173-179, wherein the therapeutic agent is a known drug or an FDA-approved drug.

181. The method of claim 179 or 180, wherein the protein is an antibody, or an antibody variant.

182. The method of claim 179 or 180, wherein the nucleic acid is an mRNA, an antisense RNA, an miRNA, an siRNA, an RNA aptamer, a double stranded RNA (dsRNA), a short hairpin RNA (shRNA), or an antisense oligonucleotide (ASO).

183. The method of any one of claims 173-182, wherein the disease or disorder is a genetic disease, a proliferative disease, an inflammatory disease, an autoimmune disease, a liver disease, a spleen disease, a lung disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, a central nervous system (CNS) disorder, or a cardiovascular disease.

184. A pair of probes comprising a first probe and a second probe, wherein:
i) the first probe comprises an oligonucleotide portion that is complementary to the second probe, an oligonucleotide portion that is complementary to an RNA of interest, and an oligonucleotide barcode sequence; and
ii) the second probe comprises a portion that recognizes an epitranscriptomic RNA modification and an oligonucleotide portion that is complementary to a portion of the first probe.

185. The pair of probes of claim 184, wherein the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), or 5-methylcytosine (m⁵C).

186. The pair of probes of claim 184, wherein the epitranscriptomic RNA modification is N⁶-methyladenosine (m⁶A).

187. The pair of probes of any one of claims 184-186, wherein the portion of the second probe that recognizes the epitranscriptomic RNA modification comprises an agent that binds an antibody, or an antibody variant.

188. The pair of probes of any one of claims 184-187, wherein the portion of the second probe that recognizes the epitranscriptomic RNA modification comprises a protein.

189. The pair of probes of claim 188, wherein the protein comprises PAPG.

190. The pair of probes of claim 189, wherein PAPG recognizes and binds an antibody that recognizes an epitranscriptomic RNA modification.

191. The pair of probes of any one of claims 184-187, wherein the portion of the second probe that recognizes the epitranscriptomic RNA modification comprises an antibody, or an antibody variant.

192. The pair of probes of claim 191, wherein the antibody is a secondary antibody.

193. The pair of probes of claim 192, wherein the secondary antibody binds a primary antibody that recognizes the epitranscriptomic RNA modification.

194. The pair of probes of claim 193, wherein the primary antibody is an anti-m⁶A antibody.

195. The pair of probes of any one of claims 184-194, wherein the portion of the second probe that recognizes the epitranscriptomic RNA modification comprises an agent that binds the epitranscriptomic RNA modification directly.

196. The pair of probes of claim 195, wherein the agent that binds the epitranscriptomic RNA modification directly comprises a protein.

197. The pair of probes of claim 196, wherein the protein is an m6A-specific YTH domain protein.

198. The pairs of probes of claim 197, wherein the agent that binds the epitranscriptomic RNA modification directly comprises an antibody, or an antibody variant.

199. The pair of probes of any one of claims 184-198, wherein the oligonucleotide portion of the first probe that is complementary to the second probe is about 3-20, about 4-19, about 5-18, about 6-17, about 7-16, about 8-15, about 9-14, about 10-13, or about 11-12 nucleotides in length.

200. The pair of probes of any one of claims 184-199, wherein the oligonucleotide portion of the first probe that is complementary to the second probe is about 3, about 4, about 5, about 6, about 7, about 8, about 9, about 10, about 11, about 12, about 13, about 14, about 15, about 16, about 17, about 18, about 19, or about 20 nucleotides in length.

201. The pair of probes of any one of claims 184-200, wherein the oligonucleotide portion of the first probe that is complementary to the second probe is split between the 5′ end and the 3′ end of the first probe.

202. The pair of probes of any one of claims 184-201, wherein the oligonucleotide portion of the first probe that is complementary to the RNA of interest is about 10-30, about 11-29, about 12-28, about 13-27, about 14-26, about 15-25, about 16-24, about 17-23, about 18-22, or about 19-21 nucleotides in length.

203. The pair of probes of any one of claims 184-202, wherein the oligonucleotide portion of the first probe that is complementary to the RNA of interest is about 10, about 11, about 12, about 13, about 14, about 15, about 16, about 17, about 18, about 19, about 20, about 21, about 22, about 23, about 24, about 25, about 26, about 27, about 28, about 29, about 30, or more than 30 nucleotides in length.

204. The pair of probes of any one of claims 184-203, wherein the oligonucleotide barcode sequence of the first probe is about 1-10, about 2-9, about 3-8, about 4-7, or about 5-6 nucleotides in length.

205. The pair of probes of any one of claims 184-204, wherein the oligonucleotide barcode sequence of the first probe is about 1, about 2, about 3, about 4, about 5, about 6, about 7, about 8, about 9, or about 10 nucleotides in length.

206. The pair of probes of any one of claims 184-205, wherein each portion of the first probe and the second probe is connected by an optional linker.

207. The pair of probes of claim 206, wherein each instance of the optional linker is independently 1, 2, 3, 4, 5, 6, 7, 8, 9, or 10 nucleotides long.

208. The pair of probes of any one of claims 184-207, wherein the first probe comprises the structure:
5′-[portion complementary to second probe]-[portion complementary to RNA of interest]-[barcode sequence]-[portion complementary to second probe]-3′;
5′-[portion complementary to second probe]-[barcode sequence]-[portion complementary to RNA of interest]-[portion complementary to second probe]-3′;
5′-[portion complementary to second probe]-[portion complementary to RNA of interest]-[barcode sequence]-3′;
5′-[portion complementary to second probe]-[barcode sequence]-[portion complementary to RNA of interest]-3′;
5′-[portion complementary to RNA of interest]-[barcode sequence]-[portion complementary to second probe]-3′; or
5′-[barcode sequence]-[portion complementary to RNA of interest]-[portion complementary to second probe]-3′,
wherein each instance of ]-[comprises an optional linker.

209. The pair of probes of any one of claims 184-208, wherein the second probe comprises the structure:
5′-[portion recognizing epitranscriptomic RNA modification]-[portion complementary to first probe]-3′,
wherein each instance of ]-[comprises an optional linker.

210. The pair of probes of any one of claims 184-209, wherein the oligonucleotide portions of the first probe and the second probe comprise DNA.

211. A set of probes comprising a first probe, a second probe, and a third probe, wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an epitranscriptomic RNA modification and an oligonucleotide portion that is complementary to a portion of the first probe; and
iii) the third probe comprises an oligonucleotide portion that is complementary to the RNA of interest and an oligonucleotide portion that is complementary to a portion of the first probe.

212. The set of probes of claim 211, wherein the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), or 5-methylcytosine (m⁵C).

213. The set of probes of claim 211, wherein the epitranscriptomic RNA modification is N⁶-methyladenosine (m⁶A).

214. The set of probes of any one of claims 211-213, wherein the portion of the second probe that recognizes the epitranscriptomic RNA modification comprises an agent that binds an antibody, or an antibody variant.

215. The set of probes of any one of claims 211-214, wherein the portion of the second probe that recognizes the epitranscriptomic RNA modification comprises a protein.

216. The set of probes of claim 215, wherein the protein comprises PAPG.

217. The set of probes of claim 216, wherein PAPG recognizes and binds an antibody that recognizes an epitranscriptomic RNA modification.

218. The set of probes of any one of claims 211-214, wherein the portion of the second probe that recognizes the epitranscriptomic RNA modification comprises an antibody, or an antibody variant.

219. The set of probes of claim 218, wherein the antibody is a secondary antibody.

220. The set of probes of claim 219, wherein the secondary antibody recognizes a primary antibody that recognizes the epitranscriptomic RNA modification.

221. The set of probes of claim 220, wherein the cell is contacted with the primary antibody prior to being contacted with the one or more pairs of probes.

222. The set of probes of claim 220 or 221, wherein the primary antibody is an anti-m⁶A antibody, an anti-m¹A antibody, an anti-pseudouridine antibody, an anti-m⁶Am antibody, an anti-m⁷G antibody, an anti-ac⁴C antibody, an anti-Nm antibody, or an anti-m⁵C antibody.

223. The set of probes of any one of claims 211-222, wherein the portion of the second probe that recognizes the epitranscriptomic RNA modification comprises an agent that binds the epitranscriptomic RNA modification directly.

224. The set of probes of claim 223, wherein the agent that binds the epitranscriptomic RNA modification directly comprises a protein.

225. The set of probes of claim 224, wherein the protein is an m⁶A-specific YTH domain protein.

226. The set of probes of claim 223, wherein the agent that binds the epitranscriptomic RNA modification directly comprises an antibody, or an antibody variant.

227. The set of probes of any one of claims 211-226, wherein the second probe further comprises a polymerization blocker.

228. The set of probes of claim 227, wherein the polymerization blocker is located at the 3′ end of second probe.

229. The set of probes of claim 227 or 228, wherein the polymerization blocker comprises an inverted nucleic acid residue.

230. The set of probes of claim 229, wherein the inverted nucleic acid residue is an inverted thymine residue.

231. The set of probes of any one of claims 211-230, wherein the oligonucleotide barcode sequence of the first probe is about 1-10, about 2-9, about 3-8, about 4-7, or about 5-6 nucleotides in length.

232. The set of probes of any one of claims 211-231, wherein the oligonucleotide barcode sequence of the first probe is about 1, about 2, about 3, about 4, about 5, about 6, about 7, about 8, about 9, or about 10 nucleotides in length.

233. The set of probes of any one of claims 211-232, wherein the first and third oligonucleotide probes are complementary to different portions of the RNA of interest.

234. The set of probes of any one of claims 211-233, wherein the first probe comprises the structure:
5′-[portion complementary to second probe]-[oligonucleotide barcode sequence]-[portion complementary to RNA of interest]-[portion complementary to third probe]-[portion complementary to second probe]-3′, wherein each instance of]-[represents an optional linker.

235. The set of probes any one of claims 211-234, wherein the portion of the first probe that is complementary to a portion of the second probe is 4-20, 5-19, 6-18, 7-17, 8-16, 9-15, 10-14, or 11-13 nucleotides in length.

236. The set of probes of any one of claims 211-235, wherein the portion of the first probe that is complementary to a portion of the second probe is 4, 5, 6, 7, 8, 9, 10, 11, 12, 13, 14, 15, 16, 17, 18, 19, or 20 nucleotides in length.

237. The set of probes of any one of claims 211-236, wherein the portion of the first probe that is complementary to a portion of the second probe is split between the 5′ end and the 3′ end of the first probe.

238. The set of probes any one of claims 211-237, wherein the portion of the first probe that is complementary to a portion of the third probe is 5-15, 6-14, 7-13, 8-12, or 9-11 nucleotides in length.

239. The set of probes of any one of claims 211-238, wherein the portion of the first probe that is complementary to a portion of the third probe is 5, 6, 7, 8, 9, 10, 11, 12, 13, 14, or 15 nucleotides in length.

240. The set of probes any one of claims 211-239, wherein the portion of the first probe that is complementary to the RNA of interest is 10-30, 11-29, 12-28, 13-27, 14-26, 15-25, 16-24,17-23, 18-22, or 19-21 nucleotides in length.

241. The set of probes any one of claims 211-240, wherein the portion of the first probe that is complementary to the RNA of interest is 10, 11, 12, 13, 14, 15, 16, 17, 18, 19, 20, 21, 22, 23, 24, 25, 26, 27, 28, 29, or 30 nucleotides in length.

242. The set of probes of any one of claims 211-241, wherein the second probe comprises the structure:
5′-[portion recognizing epitranscriptomic RNA modification]-[portion complementary to first probe]-3′, wherein]-[represents an optional linker.

243. The set of probes of any one of claims 211-242, wherein the portion of the second probe that is complementary to a portion of the first probe is 4-20, 5-19, 6-18, 7-17, 8-16, 9-15, 10-14, or 11-13 nucleotides in length.

244. The set of probes of any one of claims 211-243, wherein the portion of the second probe that is complementary to a portion of the first probe is 4, 5, 6, 7, 8, 9, 10, 11, 12, 13, 14, 15, 16, 17, 18, 19, or 20 nucleotides in length.

245. The set of probes of any one of claims 211-244, wherein the third probe comprises the structure:
5′-[portion complementary to RNA of interest]-[portion complementary to first probe]-3′, wherein ]-[represents an optional linker.

246. The set of probes of any one of claims 211-245, wherein the portion of the third probe that is complementary to the RNA of interest is 10-30, 11-29, 12-28, 13-27, 14-26, 15-25, 16-24, 17-23, 18-22, or 19-21 nucleotides in length.

247. The set of probes of any one of claims 211-246, wherein the portion of the third probe that is complementary to the RNA of interest is 10, 11, 12, 13, 14, 15, 16, 17, 18, 19, 20, 21, 22, 23, 24, 25, 26, 27, 28, 29, or 30 nucleotides in length.

248. The set of probes of any one of claims 211-247, wherein the portion of the third probe that is complementary to a portion of the first probe is 5-15, 6-14, 7-13, 8-12, or 9-11 nucleotides in length.

249. The set of probes of any one of claims 211-248, wherein the portion of the third probe that is complementary to a portion of the first probe is 5, 6, 7, 8, 9, 10, 11, 12, 13, 14, or 15 nucleotides in length.

250. A set of probes comprising a first probe, a second probe, and a third probe, wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an RNA-binding protein and an oligonucleotide portion that is complementary to a portion of the first probe; and
iii) the third probe comprises an oligonucleotide portion that is complementary to the RNA of interest and an oligonucleotide portion that is complementary to a portion of the first probe.

251. The set of probes of claim 250, wherein the RNA-binding protein comprises a YTH family protein (e.g., YTHDF1, YTHDF2, YTHDF3, YTHDC1, or YTHDC2), an IGF2BP family protein (e.g., IGF2BP1, IGF2BP2, or IGF2BP3), or FMR1.

252. The set of probes of claim 250 or 251, wherein the portion of the second probe that recognizes the RNA-binding protein comprises an agent that binds an antibody, or an antibody variant.

253. The set of probes of any one of claims 250-252, wherein the portion of the second probe that recognizes the RNA-binding protein comprises a protein.

254. The set of probes of claim 253, wherein the protein recognizes and binds an antibody that recognizes an RNA-binding protein.

255. The set of probes of any one of claims 250-252, wherein the portion of the second probe that recognizes the RNA-binding protein comprises an antibody, or an antibody variant.

256. The set of probes of claim 255, wherein the antibody is a secondary antibody.

257. The set of probes of claim 256, wherein the secondary antibody recognizes a primary antibody that recognizes the RNA-binding protein.

258. The set of probes of claim 257, wherein the cell is contacted with the primary antibody prior to being contacted with the one or more pairs of probes.

259. The set of probes of any one of claims 250-258, wherein the portion of the second probe that recognizes the RNA-binding protein comprises an agent that binds the RNA-binding protein directly.

260. The set of probes of claim 259, wherein the agent that binds the RNA-binding protein directly comprises a protein.

261. The set of probes of claim 259, wherein the agent that binds the RNA-binding protein directly comprises an antibody, or an antibody variant.

262. The set of probes of any one of claims 250-261, wherein the second probe further comprises a polymerization blocker.

263. The set of probes of claim 262, wherein the polymerization blocker is located at the 3′ end of second probe.

264. The set of probes of claim 262 or 263, wherein the polymerization blocker comprises an inverted nucleic acid residue.

265. The set of probes of claim 264, wherein the inverted nucleic acid residue is an inverted thymine residue.

266. The set of probes of any one of claims 250-265, wherein the oligonucleotide barcode sequence of the first probe is about 1-10, about 2-9, about 3-8, about 4-7, or about 5-6nucleotides in length.

267. The set of probes of any one of claims 250-266, wherein the oligonucleotide barcode of the first probe is about 1, about 2, about 3, about 4, about 5, about 6, about 7, about 8, about 9, or about 10 nucleotides in length.

268. The set of probes of any one of claims 250-267, wherein the first and third oligonucleotide probes are complementary to different portions of the RNA of interest.

269. The set of probes of any one of claims 250-268, wherein the first probe comprises the structure:
5′-[portion complementary to second probe]-[oligonucleotide barcode sequence]-[portion complementary to RNA of interest]-[portion complementary to third probe]-[portion complementary to second probe]-3′, wherein each instance of ]-[comprises an optional linker.

270. The set of probes of any one of claims 250-269, wherein the portion of the first probe that is complementary to a portion of the second probe is 4-20, 5-19, 6-18, 7-17, 8-16, 9-15, 10-14, or 11-13 nucleotides in length.

271. The set of probes of any one of claims 250-270, wherein the portion of the first probe that is complementary to a portion of the second probe is 4, 5, 6, 7, 8, 9, 10, 11, 12, 13, 14, 15, 16, 17, 18, 19, or 20 nucleotides in length.

272. The set of probes of any one of claims 250-271, wherein the portion of the first probe that is complementary to a portion of the second probe is split between the 5′ end and the 3′ end of the first probe.

273. The set of probes of any one of claims 250-272, wherein the portion of the first probe that is complementary to a portion of the third probe is 5-15, 6-14, 7-13, 8-12, or 9-11 nucleotides in length.

274. The set of probes of any one of claims 250-273, wherein the portion of the first probe that is complementary to a portion of the third probe is 5, 6, 7, 8, 9, 10, 11, 12, 13, 14, or 15 nucleotides in length.

275. The set of probes of any one of claims 250-274, wherein the portion of the first probe that is complementary to the RNA of interest is 10-30, 11-29, 12-28, 13-27, 14-26, 15-25, 16-24, 17-23, 18-22, or 19-21 nucleotides in length.

276. The set of probes of any one of claims 250-275, wherein the portion of the first probe that is complementary to the RNA of interest is 10, 11, 12, 13, 14, 15, 16, 17, 18, 19, 20, 21, 22, 23, 24, 25, 26, 27, 28, 29, or 30 nucleotides in length.

277. The set of probes of any one of claims 250-276, wherein the second probe comprises the structure:
5′-[portion recognizing RNA-binding protein]-[portion complementary to first probe]-3′, wherein]-[represents an optional linker.

278. The set of probes of any one of claims 250-277, wherein the portion of the second probe that is complementary to a portion of the first probe is 4-20, 5-19, 6-18, 7-17, 8-16, 9-15, 10-14, or 11-13 nucleotides in length.

279. The set of probes of any one of claims 250-278, wherein the portion of the second probe that is complementary to a portion of the first probe is 4, 5, 6, 7, 8, 9, 10, 11, 12, 13, 14, 15, 16, 17, 18, 19, or 20 nucleotides in length.

280. The set of probes of any one of claims 250-279, wherein the third probe comprises the structure:
5′-[portion complementary to RNA of interest]-[portion complementary to first probe]-3′, wherein]-[comprises an optional linker.

281. The set of probes of any one of claims 250-280, wherein the portion of the third probe that is complementary to the RNA of interest is 10-30, 11-29, 12-28, 13-27, 14-26, 15-25, 16-24, 17-23, 18-22, or 19-21 nucleotides in length.

282. The set of probes of any one of claims 250-281, wherein the portion of the third probe that is complementary to the RNA of interest is 10, 11, 12, 13, 14, 15, 16, 17, 18, 19, 20, 21, 22, 23, 24, 25, 26, 27, 28, 29, or 30 nucleotides in length.

283. The set of probes of any one of claims 250-282, wherein the portion of the third probe that is complementary to a portion of the first probe is 5-15, 6-14, 7-13, 8-12, or 9-11 nucleotides in length.

284. The set of probes of any one of claims 250-283, wherein the portion of the third probe that is complementary to a portion of the first probe is 5, 6, 7, 8, 9, 10, 11, 12, 13, 14, or 15 nucleotides in length.

285. A plurality of probes comprising multiple pairs of probes of any one of claims 184-210 or multiple sets of probes of any one of claims 211-284, wherein each pair of probes or set of probes comprises oligonucleotide portions that are complementary to a different RNA of interest.

286. The plurality of probes of claim 285, wherein the plurality comprises more than 1, more than 2, more than 3, more than 4, more than 5, more than 10, more than 20, more than 30, more than 40, more than 50, more than 100, more than 200, more than 500, more than 1000, more than 2000, or more than 3000 pairs of probes or sets of probes.

287. A kit comprising the pair of probes of any one of claims 184-210 or the set of probes of any one of claims 211-284.

288. The kit of claim 287, wherein the kit comprises multiple pairs of probes of any one of claims 184-210 or multiple sets of probes of any one of claims 211-284, wherein each pair of probes or set of probes comprises oligonucleotide portions that are complementary to a different RNA of interest.

289. The kit of claim 288, wherein the kit comprises more than 1, more than 2, more than 3, more than 4, more than 5, more than 10, more than 20, more than 30, more than 40, more than 50, more than 100, more than 200, more than 500, more than 1000, more than 2000, or more than 3000 pairs of probes or sets of probes.

290. The kit of any one of claims 287-289 further comprising cells.

291. The kit of any one of claims 287-290 further comprising one or more enzymes.

292. The kit of claim 291, wherein the one or more enzymes comprise a ligase.

293. The kit of claim 291 or 292, wherein the one or more enzymes comprise a polymerase.

294. The kit of any one of claims 287-293 further comprising amine-modified nucleotides.

295. The kit of any one of claims 287-294 further comprising reagents and monomers for preparing a polymeric matrix.

296. A system for profiling epitranscriptomic RNA modifications in a cell comprising
a) a cell;
b) one or more pairs of probes comprising a first probe and a second probe, wherein:
i) the first probe comprises an oligonucleotide portion that is complementary to the second probe, an oligonucleotide portion that is complementary to an RNA of interest, and an oligonucleotide barcode sequence; and
ii) the second probe comprises a portion that recognizes an epitranscriptomic RNA modification and an oligonucleotide portion that is complementary to a portion of the first probe;
c) a microscope; and
d) a computer.

297. The system of claim 296, wherein the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N-acetylcytidine (ac⁴C), 2′-O-methylation (Nm), or 5-methylcytosine (m⁵C).

298. The system of claim 296, wherein the epitranscriptomic RNA modification is N⁶-methyladenosine (m⁶A).

299. The system of any one of claims 296-298, wherein the portion of the second probe that recognizes the epitranscriptomic RNA modification comprises an agent that binds an antibody, or an antibody variant.

300. The system of any one of claims 296-299, wherein the portion of the second probe that recognizes the epitranscriptomic RNA modification comprises an antibody, or an antibody variant.

301. The system of claim 300, wherein the antibody is a secondary antibody.

302. The system of claim 301, wherein the secondary antibody binds a primary antibody that recognizes the epitranscriptomic RNA modification.

303. The system of claim 302, wherein the primary antibody is an anti-m⁶A antibody, an anti-m¹A antibody, an anti-pseudouridine antibody, an anti-m⁶Am antibody, an anti-m⁷G antibody, an anti-ac⁴C antibody, an anti-Nm antibody, or an anti-m⁵C antibody.

304. The system of any one of claims 296-303, wherein the portion of the second probe that recognizes the epitranscriptomic RNA modification comprises an agent that binds the epitranscriptomic RNA modification.

305. The system of claim 304, wherein the agent that binds the epitranscriptomic RNA modification comprises a protein.

306. The system of claim 305, wherein the protein is an m⁶A-specific YTH domain protein.

307. The system of any one of claims 296-306, wherein the oligonucleotide portion of the first probe that is complementary to the second probe is about 3-20, about 4-19, about 5-18, about 6-17, about 7-16, about 8-15, about 9-14, about 10-13, or about 11-12 nucleotides in length.

308. The system of any one of claims 296-307, wherein the oligonucleotide portion of the first probe that is complementary to the second probe is about 3, about 4, about 5, about 6, about 7, about 8, about 9, about 10, about 11, about 12, about 13, about 14, about 15, about 16, about 17, about 18, about 19, or about 20 nucleotides in length.

309. The system any one of claims 296-308, wherein the oligonucleotide portion of the first probe that is complementary to the second probe is split between the 5′ end and the 3′ end of the first probe.

310. The system of any one of claims 296-309, wherein the oligonucleotide portion of the first probe that is complementary to the RNA of interest is about 10-30, about 11-29, about 12-28, about 13-27, about 14-26, about 15-25, about 16-24, about 17-23, about 18-22, or about 19-21 nucleotides in length.

311. The system of any one of claims 296-310, wherein the oligonucleotide portion of the first probe that is complementary to the RNA of interest is about 10, about 11, about 12, about 13, about 14, about 15, about 16, about 17, about 18, about 19, about 20, about 21, about 22, about 23, about 24, about 25, about 26, about 27, about 28, about 29, about 30, or more than 30 nucleotides in length.

312. The system of any one of claims 296-311, wherein the oligonucleotide barcode sequence of the first probe is about 1-10, about 2-9, about 3-8, about 4-7, or about 5-6 nucleotides in length.

313. The system of any one of claims 296-312, wherein the oligonucleotide barcode sequence of the first probe is about 1, about 2, about 3, about 4, about 5, about 6, about 7, about 8, about 9, or about 10 nucleotides in length.

314. The system of any one of claims 296-313, wherein each portion of the first probe and the second probe is connected by an optional linker.

315. The system of claim 314, wherein each instance of the optional linker is independently 1, 2, 3, 4, 5, 6, 7, 8, 9, or 10 nucleotides long.

316. The system of any one of claims 296-315, wherein the first probe comprises the structure:
5′-[portion complementary to second probe]-[portion complementary to RNA of interest]-[barcode sequence]-[portion complementary to second probe]-3′;
5′-[portion complementary to second probe]-[barcode sequence]-[portion complementary to RNA of interest]-[portion complementary to second probe]-3′;
5′-[portion complementary to second probe]-[portion complementary to RNA of interest]-[barcode sequence]-3′;
5′-[portion complementary to second probe]-[barcode sequence]-[portion complementary to RNA of interest]-3′;
5′-[portion complementary to RNA of interest]-[barcode sequence]-[portion complementary to second probe]-3′; or
5′-[barcode sequence]-[portion complementary to RNA of interest]-[portion complementary to second probe]-3′,
wherein each instance of ]-[comprises an optional linker.

317. The system of any one of claims 296-316, wherein the second probe comprises the structure:
5′-[portion recognizing epitranscriptomic RNA modification]-[portion complementary to first probe]-3′,
wherein each instance of]-[comprises an optional linker.

318. The system of any one of claims 296-317, wherein the oligonucleotide portions of the first probe and the second probe comprise DNA.

319. A system comprising:
a) a cell;
b) one or more sets of probes comprising a first probe, a second probe, and a third probe, wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an epitranscriptomic RNA modification and an oligonucleotide portion that is complementary to a portion of the first probe; and
iii) the third probe comprises an oligonucleotide portion that is complementary to the RNA of interest and an oligonucleotide portion that is complementary to a portion of the first probe;
c) a microscope; and
d) a computer.

320. A system comprising:
a) a cell;
b) one or more sets of probes comprising a first probe, a second probe, and a third probe, wherein:
i) the first probe comprises an oligonucleotide portion that is complementary to a portion of the second probe, an oligonucleotide barcode sequence, an oligonucleotide portion that is complementary to an RNA of interest, and an oligonucleotide portion that is complementary to a portion of the third probe;
ii) the second probe comprises a portion that recognizes an RNA-binding protein and an oligonucleotide portion that is complementary to a portion of the first probe; and
iii) the third probe comprises an oligonucleotide portion that is complementary to the RNA of interest and an oligonucleotide portion that is complementary to a portion of the first probe;
c) a microscope; and
d) a computer.

321. A probe comprising a portion that recognizes an epitranscriptomic RNA modification and an oligonucleotide portion that is complementary to a portion of another probe.

322. The probe of claim 321, wherein the epitranscriptomic RNA modification is N⁶-methyladenosine (m⁶A), N¹-methyladenosine (m¹A), pseudouridine, N⁶,2′-O-dimethyladenosine (m⁶Am), 7-methylguanosine (m⁷G), N⁴-acetylcytidine (ac⁴C), 2′-O-methylation (Nm), or 5-methylcytosine (m⁵C).

323. The probe of claim 322, wherein the epitranscriptomic RNA modification is N⁶-methyladenosine (m⁶A).

324. The probe any one of claims 321-323, wherein the portion of the probe that recognizes the epitranscriptomic RNA modification comprises an agent that binds an antibody, or an antibody variant.

325. The probe of any one of claims 321-324, wherein the portion of the probe that recognizes the epitranscriptomic RNA modification comprises an antibody, or an antibody variant.

326. The probe of claim 325, wherein the antibody is a secondary antibody.

327. The probe of claim 326, wherein the secondary antibody binds a primary antibody that recognizes the epitranscriptomic RNA modification.

328. The probe of claim 327, wherein the primary antibody is an anti-m⁶A antibody.

329. The probe of any one of claims 321-328, wherein the portion of the probe that recognizes the epitranscriptomic RNA modification comprises an agent that binds the epitranscriptomic RNA modification.

330. The probe of claim 329, wherein the agent that binds the epitranscriptomic RNA modification comprises a protein.

331. The probe of claim 330, wherein the protein is an m⁶A-specific YTH domain protein.

332. The probe of claim 331, wherein the agent that binds the epitranscriptomic RNA modification comprises an antibody, or an antibody variant.

333. The probe of any one of claims 321-332, wherein the oligonucleotide portion of the probe that is complementary to a portion of another probe is about 3, about 4, about 5, about 6, about 7, about 8, about 9, about 10, about 11, about 12, about 13, about 14, about 15, about 16, about 17, about 18, about 19, or about 20 nucleotides in length.

334. The probe of any one of claims 321-333, wherein the oligonucleotide portion of the probe that is complementary to a portion of another probe is split between the 5′ end and the 3′ end of the first probe.

335. The probe of any one of claims 321-334, wherein each portion of the probe is connected by an optional linker.

336. The probe of claim 335, wherein each instance of the optional linker is independently 1, 2, 3, 4, 5, 6, 7, 8, 9, or 10 nucleotides long.

337. The probe of any one of claims 321-336, wherein the probe comprises the structure:
5′-[portion recognizing epitranscriptomic RNA modification]-[portion complementary to another probe]-3′,
wherein each instance of]-[comprises an optional linker.

338. The probe of any one of claims 321-337, wherein the oligonucleotide portion of the probe comprises DNA.
